SEQUENCE LISTING (22 sequences)

SEQ ID NO: 1 - protein, 8 aa, synthetic construct
AQTKILPR

SEQ ID NO: 2 - protein, 15 aa, synthetic construct
GGGGSGGGGSGGGGS

SEQ ID NO: 3 - protein, 10 aa, synthetic construct
YVYVADVAAK

SEQ ID NO: 4 - protein, 8 aa, synthetic construct
YEMFNDKS

SEQ ID NO: 5 - protein, 9 aa, synthetic construct
YEMFNDKSF

SEQ ID NO: 6 - protein, 11 aa, synthetic construct
HROEIFSHDFJ

SEQ ID NO: 7 - protein, 10 aa, synthetic construct
FJIEJFOESS

SEQ ID NO: 8 - protein, 10 aa, synthetic construct
NEIOREIREI

SEQ ID NO: 9 - protein, 15 aa, synthetic construct
JFKSIFEMMSJDSSU

SEQ ID NO: 10 - protein, 13 aa, synthetic construct
KNFLENFIESOFI

SEQ ID NO: 11 - protein, 15 aa, synthetic construct
FOEIFNDKSLDKFJI

SEQ ID NO: 12 - protein, 9 aa, synthetic construct
QCEIOWARE

SEQ ID NO: 13 - protein, 8 aa, synthetic construct
FIEUHFWI

SEQ ID NO: 14 - protein, 12 aa, synthetic construct
FEWRHRJTRUJR

SEQ ID NO: 15 - protein, 9 aa, synthetic construct
QIEJOEIJE

SEQ ID NO: 16 - protein, 14 aa, synthetic construct
FJELFISBOSJFIE

SEQ ID NO: 17 - protein, 11 aa, synthetic construct
IEFROEIFJEF

SEQ ID NO: 18 - protein, 9 aa, synthetic construct
IEFROEIFJ

SEQ ID NO: 19 - protein, 9 aa, synthetic construct
EFROEIFJE

SEQ ID NO: 20 - protein, 9 aa, synthetic construct
FROEIFJEF

SEQ ID NO: 21 - protein, 9 aa, synthetic construct
FEGRKUOOI

SEQ ID NO: 22 - protein, 14 aa, synthetic construct
PJFIOEJOIJGEIO

(19) United States
(12) Patent Application Publication
(10) Pub. No.: US 2025/0273288 A1
(43) Pub. Date: Aug. 28, 2025

(54) ANTIGEN PREDICTIONS FOR INFECTIOUS DISEASE-DERIVED EPITOPES

(71) Applicant: Gritstone Bio, Inc., Emeryville, CA (US)

(72) Inventors: Joshua Klein, Brookline, MA (US); Minh Duc Cao, Belmont, MA (US)

(21) Appl. No.: 18/908,082

(22) Filed: Oct. 7, 2024

Related U.S. Application Data
(63) Continuation of application No. PCT/US2023/065518, filed on Apr. 7, 2023.
(60) Provisional application No. 63/329,259, filed on Apr. 8, 2022.

(51) Int. Cl. G16B 15/30; G16B 40/10; G16B 40/20; G16H 20/17

(52) U.S. Cl. CPC G16B 15/30; G16B 40/10; G16B 40/20; G16H 20/17

(57) ABSTRACT

Disclosed herein is a system and methods for determining the alleles, antigens, and infectious disease-based vaccine composition as determined on the basis of a patient's expressed HLA alleles. Additionally described herein are unique infectious disease-derived vaccines.

### CROSS-REFERENCE TO RELATED APPLICATIONS

This application is a continuation of International Patent Application No. PCT/US2023/065518, filed Apr. 7, 2023, which claims the benefit of and priority to U.S. Provisional Patent Application No. 63/329,259 filed Apr. 8, 2022, the entire disclosure of which is hereby incorporated by reference in its entirety for all purposes.

### SEQUENCE LISTING

The instant application contains a Sequence Listing which has been submitted electronically in .XML. format and is hereby incorporated by reference in its entirety. Said .XML copy, created on 16 Sep., 2024, is named GSO-113WO.XML and is 20,182 bytes in size.

### BACKGROUND

Therapeutic vaccines for viral infections holds great promise for personalized therapy. Accurate predictions of viral epitopes that are likely presented by HLA alleles can be valuable for developing therapeutic vaccines. A therapeutic vaccine that includes the predicted viral epitopes can be more effective at eliciting an anti-viral immune response. However, unlike prior efforts in cancer where there exist large mass spectrometry (MS) human immunopeptidomic datasets, such MS immunopeptidomic datasets are lacking for infectious disease-derived antigens. This represents a significant limitation in constructing models for predicting likely presentation of viral epitopes. Thus, there is a need for new models that are able to effectively leverage other types of data (e.g., in addition to MS immunopeptidomic datasets).

### SUMMARY

Disclosed herein is an optimized approach for identifying and selecting infectious disease-derived antigens for personalized infectious disease (ID) vaccines. Generally, the approach involves applying a multi-part presentation model to generate per-allele presentation likelihoods representing whether individual HLA alleles (e.g., HLA alleles expressed by a patient) are likely to present an epitope (e.g., an infectious disease-derived epitope). In various embodiments, the multi-part presentation model comprises a first part that comprises a pan-allele model portion (also referred to as a pan-specific model portion) and a second part that comprises one or more allele-specific model portions, also referred to herein as per-allele models. Generally, the pan-allele model portion allows sharing of information across similar alleles, thereby enabling the pan-allele model portion to generate presentation likelihoods across various HLA alleles, including HLA alleles that the pan-allele model portion had not previously encountered. Each of the allele-specific model portions represents a network model that generates a presentation likelihood for a specific HLA allele. Thus, both the pan-allele model portion and the allele-specific model portions generate per-allele presentation likelihoods. By combining the outputs of the pan-allele model portion and the allele-specific model portions, the multi-part presentation model outputs improved per-allele presentation likelihoods for individual peptide sequences (e.g., infectious disease-derived peptide sequences).

In various embodiments, the multi-part presentation model is trained using training data derived from one of 1) binding affinity data between training peptide sequences and HLA alleles and 2) eluted peptide data from mass spectrometry representing presentation of training peptide sequences and HLA alleles. In particular embodiments, the multi-part presentation model is trained using training data derived from both of 1) binding affinity data between training peptide sequences and HLA alleles and 2) eluted peptide data from mass spectrometry representing presentation of training peptide sequences and HLA alleles. Here, it may be preferable to use both types of data to train the multi-part presentation model, especially in scenarios where eluted peptide data of infectious disease-derived peptide sequences generated via mass spectrometry are limited in quantity.

Disclosed herein is a method for identifying one or more infectious disease-derived antigens likely to be presented by cells of a subject, the method comprising: obtaining peptide sequences of a plurality of infectious disease-derived antigens; obtaining sequences of one or more MHC alleles of the subject; inputting the peptide sequences of the plurality of infectious disease-derived antigens and the sequences of one or more MHC alleles of the subject into a multi-part presentation model to generate a set of numerical likelihoods that the plurality of infectious disease-derived antigens are presented by the one or more MHC alleles expressed on surfaces of cells of the subject, wherein a first part of the multi-part presentation model comprises a pan-allele model portion that receives, as input, peptide sequences of one or more infectious disease-derived antigens and the sequences of one or more MHC alleles of the subject, or representations thereof, and wherein a second part of the multi-part presentation model comprises a plurality of allele-specific models that each receives, as input, the peptide sequences of the plurality of infectious disease-derived antigens, or a representation thereof; and selecting a subset of the plurality of infectious disease-derived antigens based on the set of numerical likelihoods to generate a set of selected antigens.

In various embodiments, the multi-part presentation model comprises a plurality of parameters generated using at least 1) mass spectrometry data and 2) binding affinity data determined from a plurality of samples. In various embodiments, the multi-part presentation model comprises a plurality of parameters generated using a training dataset comprising: training peptide sequences, and for one or more of the training peptide sequences, a label derived from mass spectrometry data indicating whether the training peptide sequence was presented by one or more class I MHC alleles present in a plurality of samples. In various embodiments, the training peptide sequences are identified through mass spectrometry on isolated peptides eluted from MHC alleles present in the plurality of samples. In various embodiments, the multi-part presentation model comprises a plurality of parameters generated using a training dataset comprising: for one or more of the training peptide sequences, a label derived from binding affinity data indicating whether the training peptide sequence was bound with one or more class I MHC alleles present in a plurality of samples.

In various embodiments, the training peptide sequences are of lengths within a range of k-mers where k is between 8-15, inclusive. In various embodiments, the training peptide sequences are of lengths within a range of k-mers where k is between 8-11, inclusive. In various embodiments, the training peptide sequences are infectious disease-derived training peptide sequences.

In various embodiments, the pan-allele model portion comprises a neural network. In various embodiments, a first set of layers of the neural network of the pan-allele model portion performs a dimensional reduction of the sequences of one or more MHC alleles of the subject. In various embodiments, a second set of layers of the neural network of the pan-allele model portion receives, as input, a representation of the peptide sequences of the plurality of infectious disease-derived antigens and a dimensionally reduced representation of the sequences of one or more MHC alleles of the subject. In various embodiments, the representation of the peptide sequences of the plurality of infectious disease-derived antigens is generated by encoding the peptide sequences via a one-hot encoding scheme. In various embodiments, the second set of layers of the neural network models interactions between the peptide sequences of the plurality of infectious disease-derived antigens and the sequences of one or more MHC alleles of the subject.

In various embodiments, one or more of the allele-specific models comprise a neural network. In various embodiments, the neural network of the allele-specific network receives, as input, a representation of the peptide sequences of the plurality of infectious disease-derived antigens, and outputs per-allele presentation likelihoods for an allele. In various embodiments, the representation of the peptide sequences of the plurality of infectious disease-derived antigens is generated by encoding the peptide sequences via a one-hot encoding scheme. In various embodiments, each of the allele-specific models comprise a neural network. In various embodiments, the second part of the multi-part presentation model comprise ten or more allele-specific models.

In various embodiments, a numerical likelihood of antigens is a combination of an output of the pan-allele model portion and outputs of the plurality of allele-specific models. In various embodiments, the set of numerical likelihoods are further identified by features comprising at least one of: (a) C-terminal sequences flanking the peptide sequences of the plurality of infectious disease-derived antigens; and (b) the N-terminal sequences flanking peptide sequences of the plurality of infectious disease-derived antigens.

In various embodiments, the plurality of samples comprise at least one of: (a) one or more cell lines engineered to express a single MHC class I allele; (b) one or more cell lines engineered to express a plurality of MHC class I alleles; (c) one or more human cell lines obtained or derived from a plurality of patients; (d) fresh or frozen samples obtained from a plurality of patients; and (e) fresh or frozen tissue samples obtained from a plurality of patients. In various embodiments, the cells of the subject comprise cells infected with one of a pathogen, virus, bacteria, fungus, or a parasite. In various embodiments, infectious disease-derived antigens originate from one of a pathogen, virus, bacteria, fungus, or a parasite. In various embodiments, infectious disease-derived antigens originate from an infectious disease organism selected from the group consisting of: severe acute respiratory syndrome-related coronavirus (SARS), severe acute respiratory syndrome coronavirus 2 (SARS-CoV-2), Ebola, HIV, Hepatitis B virus (HBV), influenza, Hepatitis C virus (HCV), Human papillomavirus (HPV), Cytomegalovirus (CMV), Chikungunya virus, Respiratory syncytial virus (RSV), Dengue virus, a orthymyxoviridae family virus, tuberculosis, pancorona, herpes simplex virus infection (HSV), flu, metapneumovirus (MPV), and Parainfluenza Viruses (PIVs).

Additionally disclosed herein is a method of treating a subject for an infectious disease comprising performing any of the methods disclosed herein, and further comprising obtaining a vaccine comprising the set of selected antigens, and administering the vaccine to the subject. In various embodiments, the vaccine is administered to the subject prophylactically. In various embodiments, the vaccine is administered to the subject therapeutically. Additionally disclosed herein is a method of manufacturing a vaccine, comprising performing any of the methods disclosed herein, and further comprising producing or having produced a vaccine comprising the set of selected antigens. Additionally disclosed herein is a a vaccine comprising a set of selected antigens selected by performing any one of the methods disclosed herein. Additionally disclosed herein is a method of treating a subject for an infectious disease comprising performing any one of the methods disclosed herein, and further comprising obtaining an isolated antigen binding protein exhibiting binding specificity for one or more of the selected antigens, and administering the isolated antigen binding protein to the subject. Additionally disclosed herein is a method of treating a subject for an infectious disease comprising performing any of the methods disclosed herein, and further comprising obtaining an isolated antigen binding protein exhibiting binding specificity for one or more of the selected antigens, and administering the isolated antigen binding protein to the subject. In various embodiments, the antigen binding protein is an antibody or antigen binding fragment. In various embodiments, the antigen binding protein is a T-cell receptor or a chimeric antigen receptor.

Additionally disclosed herein is a non-transitory computer readable medium comprising instructions that, when executed by a processor, cause the processor to: obtain peptide sequences of a plurality of infectious disease-derived antigens; obtain sequences of one or more MHC alleles of the subject; input the peptide sequences of the plurality of infectious disease-derived antigens and the sequences of one or more MHC alleles of the subject into a multi-part presentation model to generate a set of numerical likelihoods that the plurality of infectious disease-derived antigens are presented by the one or more MHC alleles expressed on surfaces of cells of the subject, wherein a first part of the multi-part presentation model comprises a pan-allele model portion that receives, as input, peptide sequences of one or more infectious disease-derived antigens and the sequences of one or more MHC alleles of the subject, or representations thereof, and wherein a second part of the multi-part presentation model comprises a plurality of allele-specific models that each receives, as input, the peptide sequences of the plurality of infectious disease-derived antigens, or a representation thereof; and select a subset of the plurality of infectious disease-derived antigens based on the set of numerical likelihoods to generate a set of selected antigens. In various embodiments, the multi-part presentation model comprises a plurality of parameters generated using at least 1) mass spectrometry data and 2) binding affinity data determined from a plurality of samples. In various embodiments, the multi-part presentation model comprises a plurality of parameters generated using a training dataset comprising: training peptide sequences, and for one or more of the training peptide sequences, a label derived from mass spectrometry data indicating whether the training peptide sequence was presented by one or more class I MHC alleles present in a plurality of samples. In various embodiments, the training peptide sequences are identified through mass spectrometry on isolated peptides eluted from MHC alleles present in the plurality of samples.

In various embodiments, the multi-part presentation model comprises a plurality of parameters generated using a training dataset comprising: for one or more of the training peptide sequences, a label derived from binding affinity data indicating whether the training peptide sequence was bound with one or more class I MHC alleles present in a plurality of samples. In various embodiments, the training peptide sequences are of lengths within a range of k-mers where k is between 8-15, inclusive. In various embodiments, the training peptide sequences are of lengths within a range of k-mers where k is between 8-11, inclusive.

In various embodiments, the training peptide sequences are infectious disease-derived training peptide sequences. In various embodiments, the pan-allele model portion comprises a neural network. In various embodiments, a first set of layers of the neural network of the pan-allele model portion performs a dimensional reduction of the sequences of one or more MHC alleles of the subject. In various embodiments, a second set of layers of the neural network of the pan-allele model portion receives, as input, a representation of the peptide sequences of the plurality of infectious disease-derived antigens and a dimensionally reduced representation of the sequences of one or more MHC alleles of the subject. In various embodiments, the representation of the peptide sequences of the plurality of infectious disease-derived antigens is generated by encoding the peptide sequences via a one-hot encoding scheme. In various embodiments, the second set of layers of the neural network models interactions between the peptide sequences of the plurality of infectious disease-derived antigens and the sequences of one or more MHC alleles of the subject. In various embodiments, one or more of the allele-specific models comprise a neural network. In various embodiments, the neural network of the allele-specific network receives, as input, a representation of the peptide sequences of the plurality of infectious disease-derived antigens, and outputs per-allele presentation likelihoods for an allele.

In various embodiments, the representation of the peptide sequences of the plurality of infectious disease-derived antigens is generated by encoding the peptide sequences via a one-hot encoding scheme. In various embodiments, each of the allele-specific models comprise a neural network. In various embodiments, the second part of the multi-part presentation model comprise ten or more allele-specific models. In various embodiments, a numerical likelihood of a antigens is a combination of an output of the pan-allele model portion and outputs of the plurality of allele-specific models. In various embodiments, the set of numerical likelihoods are further identified by features comprising at least one of: (a) C-terminal sequences flanking the peptide sequences of the plurality of infectious disease-derived antigens; and (b) the N-terminal sequences flanking peptide sequences of the plurality of infectious disease-derived antigens. In various embodiments, the plurality of samples comprise at least one of: (a) one or more cell lines engineered to express a single MHC class I allele; (b) one or more cell lines engineered to express a plurality of MHC class I alleles; (c) one or more human cell lines obtained or derived from a plurality of patients; (d) fresh or frozen samples obtained from a plurality of patients; and (e) fresh or frozen tissue samples obtained from a plurality of patients.

In various embodiments, the cells of the subject comprise cells infected with one of a pathogen, virus, bacteria, fungus, or a parasite. In various embodiments, infectious disease-derived antigens originate from one of a pathogen, virus, bacteria, fungus, or a parasite. In various embodiments, infectious disease-derived antigens originate from an infectious disease organism selected from the group consisting of: severe acute respiratory syndrome-related coronavirus (SARS), severe acute respiratory syndrome coronavirus 2 (SARS-CoV-2), Ebola, HIV, Hepatitis B virus (HBV), influenza, Hepatitis C virus (HCV), Human papillomavirus (HPV), Cytomegalovirus (CMV), Chikungunya virus, Respiratory syncytial virus (RSV), Dengue virus, a orthymyxoviridae family virus, tuberculosis, pancorona, herpes simplex virus infection (HSV), flu, metapneumovirus (MPV), and Parainfluenza Viruses (PIVs).

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A is an overview of an environment for identifying likelihoods of peptide presentation in patients, in accordance with an embodiment.

FIGS. 1B and 1C describe an example cassette design methodology, in accordance with an embodiment.

FIGS. 2A and 2B illustrate a method of obtaining presentation information, in accordance with an embodiment.

FIG. 3A is a high-level block diagram illustrating the computer logic components of the presentation identification system, according to one embodiment.

FIG. 3B illustrates an example set of training data, according to one embodiment.

FIG. 4A represents a flow process for implementing the multi-part presentation model, according to one embodiment.

FIG. 4B shows the network architecture of the multi-part presentation model, according to one embodiment.

FIG. 5A shows the implementation of an allele-specific model portion of the multi-part presentation model, according to one embodiment.

FIG. 5B illustrates an example network model in association with an MHC allele.

FIG. 5C illustrates an example network model shared by MHC alleles.

FIG. 5D illustrates generating a presentation likelihood for a peptide in association with an MHC allele using an example network model.

FIG. 5E illustrates generating a presentation likelihood for a peptide in association with a MHC allele using example network models.

FIG. 5F illustrates generating a presentation likelihood for a peptide in association with MHC alleles using example network models.

FIG. 5G illustrates generating a presentation likelihood for a peptide in association with MHC alleles using example network models.

FIG. 5H illustrates generating a presentation likelihood for a peptide in association with MHC alleles using example network models.

FIG. 5I illustrates generating a presentation likelihood for a peptide in association with MHC alleles using example network models.

FIG. 6A shows the implementation of a pan-allele model portion of the multi-part presentation model, according to one embodiment.

FIG. 6B illustrates an example network model shared by MHC alleles, according to an embodiment.

FIG. 6C illustrates an example network model that is not associated with an MHC allele.

FIG. 6D illustrates generating a presentation likelihood for a peptide in association with an MHC allele using an example network model shared by MHC alleles.

FIG. 7 illustrates an example computer for implementing the entities shown in FIGS. 1A and 3A.

FIG. 8A shows performance of various models for predicting presentation of HIV epitopes across the top 5 alleles.

FIG. 8B shows performance of various models for predicting presentation of Influenza A epitopes across the top 5 alleles.

FIG. 8C shows performance of various models for predicting presentation of SARS-CoV-2 epitopes across the top 5 alleles.

FIG. 9A shows precision recall curves of various models for predicting presentation of HIV epitopes across the top 25 alleles.

FIG. 9B shows precision recall curves of various models for predicting presentation of Influenza A epitopes across the top 25 alleles.

### DETAILED DESCRIPTION

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein the term “antigen” is a substance that induces an immune response. In various embodiments, a “antigen” refers to an infectious disease-derived antigen originating from one of a pathogen, virus, bacteria, fungus, or a parasite capable of causing an infectious disease. An antigen can include a polypeptide sequence or a nucleotide sequence. In various embodiments, an antigen can include a mutation, such as a frameshift or nonframeshift indel, missense or nonsense substitution, splice site alteration, splice variant, genomic rearrangement or gene fusion.

As used herein the term “antigen-based vaccine” is a vaccine construct based on one or more antigens, e.g., a plurality of antigens.

As used herein the term “coding region” is the portion(s) of a gene that encode protein.

As used herein the term “coding mutation” is a mutation occurring in a coding region.

As used herein the term “indel” is an insertion or deletion of one or more nucleic acids.

As used herein the term “epitope” is the specific portion of an antigen typically bound by an antibody or T cell receptor.

As used herein the term “immunogenic” is the ability to elicit an immune response, e.g., via T cells, B cells, or both.

As used herein the term “HLA binding affinity” “MHC binding affinity” means affinity of binding between a specific antigen and a specific MHC allele.

As used herein the term “polymorphism” is a germline variant, i.e., a variant found in all DNA-bearing cells of an individual.

As used herein the term “somatic variant” is a variant arising in non-germline cells of an individual.

As used herein the term “allele” is a version of a gene or a version of a genetic sequence or a version of a protein.

As used herein the term “HLA type” is the complement of HLA gene alleles.

As used herein the term “nonsense-mediated decay” or “NMD” is a degradation of an mRNA by a cell due to a premature stop codon.

As used herein the term “exome” is a subset of the genome that codes for proteins. An exome can be the collective exons of a genome.

As used herein the term “logistic regression” is a regression model for binary data from statistics where the logit of the probability that the dependent variable is equal to one is modeled as a linear function of the dependent variables.

As used herein the term “neural network” is a machine learning model for classification or regression consisting of multiple layers of linear transformations followed by element-wise nonlinearities typically trained via stochastic gradient descent and back-propagation.

As used herein the term “proteome” is the set of all proteins expressed and/or translated by a cell, group of cells, or individual.

As used herein the term “peptidome” is the set of all peptides presented by MHC-I or MHC-II on the cell surface. The peptidome may refer to a property of a cell or a collection of cells).

As used herein the term “ELISPOT” means Enzyme-linked immunosorbent spot assay—which is a common method for monitoring immune responses in humans and animals.

As used herein the term “tolerance or immune tolerance” is a state of immune non-responsiveness to one or more antigens, e.g. self-antigens.

As used herein the term “central tolerance” is a tolerance affected in the thymus, either by deleting self-reactive T-cell clones or by promoting self-reactive T-cell clones to differentiate into immunosuppressive regulatory T-cells (Tregs).

As used herein the term “peripheral tolerance” is a tolerance affected in the periphery by downregulating or anergizing self-reactive T-cells that survive central tolerance or promoting these T cells to differentiate into Tregs.

The term “sample” can include a single cell or multiple cells or fragments of cells or an aliquot of body fluid, taken from a subject, by means including venipuncture, excretion, ejaculation, massage, biopsy, needle aspirate, lavage sample, scraping, surgical incision, or intervention or other means known in the art.

The terms “subject” and “patient” are used interchangeably and encompass a cell, tissue, or organism, human or non-human, whether in vivo, ex vivo, or in vitro, male or female. The terms subject and patient are inclusive of mammals including humans.

The term “mammal” encompasses both humans and non-humans and includes but is not limited to humans, non-human primates, canines, felines, murines, bovines, equines, and porcines.

The term “clinical factor” refers to a measure of a condition of a subject, e.g., disease activity or severity. “Clinical factor” encompasses all markers of a subject's health status, including non-sample markers, and/or other characteristics of a subject, such as, without limitation, age and gender. A clinical factor can be a score, a value, or a set of values that can be obtained from evaluation of a sample (or population of samples) from a subject or a subject under a determined condition. A clinical factor can also be predicted by markers and/or other parameters such as gene expression surrogates.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody, camelid VHH, engineered or evolved human VH that does not require pairing to VL for solubility or activity) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

Abbreviations: MHC: major histocompatibility complex; HLA: human leukocyte antigen, or the human MHC gene locus; NGS: next-generation sequencing; PPV: positive predictive value; FFPE: formalin-fixed, paraffin-embedded; NMD: nonsense-mediated decay; DC: dendritic cell.

It should be noted that, as used in the specification and the appended claims, the singular forms “a,” “an,” and “the” include plural referents unless the context clearly dictates otherwise.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

All references, issued patents and patent applications cited within the body of the specification are hereby incorporated by reference in their entirety, for all purposes.

### II. Methods of Identifying Antigens

Methods for identifying antigens (e.g., antigens derived from an infectious disease organism) include identifying antigens from an infectious disease organism, an infection in a subject, or an infected cell of a subject that are likely to be presented on the cell surface of infected cells or immune cells, including professional antigen presenting cells such as dendritic cells, and/or are likely to be immunogenic. As an example, one such method may comprise the steps of: obtaining at least one of exome, transcriptome or whole genome infectious disease organism nucleotide sequencing and/or expression data from the an infected cell of the subject, wherein the infectious disease organism nucleotide sequencing and/or expression data is used to obtain data representing peptide sequences of each of a set of antigens (e.g., antigens derived from an infectious disease organism); inputting the peptide sequence of each antigen into one or more presentation models to generate a set of numerical likelihoods that each of the antigens is presented by one or more MHC alleles on the cell surface of an infected cell of the subject or cells present in the subject, the set of numerical likelihoods having been identified at least based on received mass spectrometry data; and selecting a subset of the set of antigens based on the set of numerical likelihoods to generate a set of selected antigens.

The presentation model, such as a multi-part presentation model, can comprise a statistical regression or a machine learning (e.g., deep learning) model trained on a set of reference data (also referred to as a training data set) comprising a set of corresponding labels, wherein the set of reference data is obtained from each of a plurality of distinct subjects where optionally some subjects can have an infection, and wherein the set of reference data comprises at least one of: data representing exome nucleotide sequences from infected tissue, data representing exome nucleotide sequences from normal tissue, data representing transcriptome nucleotide sequences from infected tissue, data representing proteome sequences from infected tissue, and data representing MHC peptidome sequences from infected tissue, and data representing MHC peptidome sequences from normal tissue. The reference data can further comprise mass spectrometry data, sequencing data, RNA sequencing data, expression profiling data, and proteomics data for single-allele cell lines engineered to express a predetermined MHC allele that are subsequently exposed to synthetic protein, normal human cell lines, and fresh and frozen primary samples, and T cell assays (e.g., ELISpot). In certain aspects, the set of reference data includes each form of reference data.

The presentation model can comprise a set of features derived at least in part from the set of reference data, and wherein the set of features comprises at least one of allele dependent-features and allele-independent features. In certain aspects each feature is included.

Methods for identifying shared antigens also include generating an output for constructing a personalized vaccine by identifying one or more antigens from one or more cells of a subject that are likely to be presented on a surface of infected cells. As an example, one such method may comprise the steps of: obtaining at least one of exome, transcriptome, or whole genome nucleotide sequencing and/or expression data from the infected cells and normal cells of the subject, wherein the nucleotide sequencing and/or expression data is used to obtain data representing peptide sequences of each of a set of antigens identified by comparing the nucleotide sequencing and/or expression data from the infected cells and the nucleotide sequencing and/or expression data from the normal cells, peptide sequence identified from the normal cells of the subject; encoding the peptide sequences of each of the antigens into a corresponding numerical vector, each numerical vector including information regarding a plurality of amino acids that make up the peptide sequence and a set of positions of the amino acids in the peptide sequence; inputting the numerical vectors, using a computer processor, into a deep learning presentation model to generate a set of presentation likelihoods for the set of antigens, each presentation likelihood in the set representing the likelihood that a corresponding antigen is presented by one or more class II MHC alleles on the surface of the infected cells of the subject, the deep learning presentation model; selecting a subset of the set of antigens based on the set of presentation likelihoods to generate a set of selected antigens; and generating the output for constructing the personalized vaccine based on the set of selected antigens.

Specific methods for identifying antigens (e.g., infectious disease organism derived antigens) are known to those skilled in the art, for example the methods described in more detail in international patent application publications WO/2017/106638, WO/2018/195357, and WO/2018/208856, each herein incorporated by reference, in their entirety, for all purposes.

A method of treating a subject having an infection is disclosed herein, comprising performing the steps of any of the antigen identification methods described herein, and further comprising obtaining an infectious disease vaccine comprising the set of selected antigens, and administering the infectious disease vaccine to the subject.

A method disclosed herein can also include identifying one or more T cells that are antigen-specific for at least one of the antigens in the subset. In some embodiments, the identification comprises co-culturing the one or more T cells with one or more of the antigens in the subset under conditions that expand the one or more antigen-specific T cells. In further embodiments, the identification comprises contacting the one or more T cells with a tetramer comprising one or more of the antigens in the subset under conditions that allow binding between the T cell and the tetramer. In even further embodiments, the method disclosed herein can also include identifying one or more T cell receptors (TCR) of the one or more identified T cells. In certain embodiments, identifying the one or more T cell receptors comprises sequencing the T cell receptor sequences of the one or more identified T cells. The method disclosed herein can further comprise genetically engineering a plurality of T cells to express at least one of the one or more identified T cell receptors; culturing the plurality of T cells under conditions that expand the plurality of T cells; and infusing the expanded T cells into the subject. In some embodiments, genetically engineering the plurality of T cells to express at least one of the one or more identified T cell receptors comprises cloning the T cell receptor sequences of the one or more identified T cells into an expression vector; and transfecting each of the plurality of T cells with the expression vector. In some embodiments, the method disclosed herein further comprises culturing the one or more identified T cells under conditions that expand the one or more identified T cells; and infusing the expanded T cells into the subject.

Also disclosed herein is an isolated T cell that is antigen-specific for at least one selected antigen in the subset.

Also disclosed herein is a methods for manufacturing an infectious disease vaccine, comprising the steps of: obtaining at least one of exome, transcriptome or whole genome infectious disease organism nucleotide sequencing and/or expression data from the infected cell of the subject, wherein the infectious disease organism nucleotide sequencing and/or expression data is used to obtain data representing peptide sequences of each of a set of antigens (e.g., where peptides are derived from any polypeptide known to or have been found to have altered expression in a infected cell or infected tissue in comparison to a normal cell or tissue); inputting the peptide sequence of each antigen into one or more presentation models to generate a set of numerical likelihoods that each of the antigens is presented by one or more MHC alleles on the cell surface of the infected cell of the subject, the set of numerical likelihoods having been identified at least based on received mass spectrometry data; and selecting a subset of the set of antigens based on the set of numerical likelihoods to generate a set of selected antigens; and producing or having produced an infectious disease vaccine comprising the set of selected antigens.

Also disclosed herein is an infectious disease vaccine including a set of selected antigens selected by performing the method comprising the steps of: obtaining at least one of exome, transcriptome or whole genome infectious disease organism nucleotide sequencing and/or expression data from the infected cell of the subject, wherein the infectious disease organism nucleotide sequencing and/or expression data is used to obtain data representing peptide sequences of each of a set of antigens, and wherein the peptide sequence of each antigen (e.g., derived from any polypeptide known to or have been found to have altered expression in a infected cell or infected tissue in comparison to a normal cell or tissue); inputting the peptide sequence of each antigen into one or more presentation models to generate a set of numerical likelihoods that each of the antigens is presented by one or more MHC alleles on the cell surface of the infected cell of the subject, the set of numerical likelihoods having been identified at least based on received mass spectrometry data; and selecting a subset of the set of antigens based on the set of numerical likelihoods to generate a set of selected antigens; and producing or having produced an infectious disease vaccine comprising the set of selected antigens.

The vaccine may include one or more of a nucleotide sequence, a polypeptide sequence, RNA, DNA, a cell, a plasmid, or a vector.

The vaccine may include one or more antigens presented on the infected cell surface.

The infectious disease vaccine may include one or more antigens that is immunogenic in the subject.

The infectious disease vaccine may not include one or more antigens that induce an autoimmune response against normal tissue in the subject.

The infectious disease vaccine may include an adjuvant.

The infectious disease vaccine may include an excipient.

A method disclosed herein may also include selecting antigens that have an increased likelihood of being presented on the infected cell surface relative to unselected antigens based on the presentation model.

A method disclosed herein may also include selecting antigens that have an increased likelihood of being capable of inducing an infectious disease organism-specific immune response in the subject relative to unselected antigens based on the presentation model.

A method disclosed herein may also include selecting antigens that have an increased likelihood of being capable of being presented to naïve T cells by professional antigen presenting cells (APCs) relative to unselected antigens based on the presentation model, optionally wherein the APC is a dendritic cell (DC).

A method disclosed herein may also include selecting antigens that have a decreased likelihood of being subject to inhibition via central or peripheral tolerance relative to unselected antigens based on the presentation model.

A method disclosed herein may also include selecting antigens that have a decreased likelihood of being capable of inducing an autoimmune response to normal tissue in the subject relative to unselected antigens based on the presentation model.

The exome or transcriptome nucleotide sequencing and/or expression data may be obtained by performing sequencing on the infected tissue.

The sequencing may be next generation sequencing (NGS) or any massively parallel sequencing approach.

The set of numerical likelihoods may be further identified by at least MHC-allele interacting features comprising at least one of: the predicted affinity with which the MHC allele and the antigen encoded peptide bind; the predicted stability of the antigen encoded peptide-MHC complex; the sequence and length of the antigen encoded peptide; the probability of presentation of antigen encoded peptides with similar sequence in cells from other individuals expressing the particular MHC allele as assessed by mass-spectrometry proteomics or other means; the expression levels of the particular MHC allele in the subject in question (e.g. as measured by RNA-seq or mass spectrometry); the overall antigen encoded peptide-sequence-independent probability of presentation by the particular MHC allele in other distinct subjects who express the particular MHC allele; the overall antigen encoded peptide-sequence-independent probability of presentation by MHC alleles in the same family of molecules (e.g., HLA-A, HLA-B, HLA-C, HLA-DQ, HLA-DR, HLA-DP) in other distinct subjects.

The set of numerical likelihoods are further identified by at least MHC-allele noninteracting features comprising at least one of: the C- and N-terminal sequences flanking the antigen encoded peptide within its source protein sequence; the presence of protease cleavage motifs in the antigen encoded peptide, optionally weighted according to the expression of corresponding proteases in the infected cells (as measured by RNA-seq or mass spectrometry); the turnover rate of the source protein as measured in the appropriate cell type; the length of the source protein, optionally considering the specific splice variants (“isoforms”) most highly expressed in the infected cells as measured by RNA-seq or proteome mass spectrometry, or as predicted from the annotation of germline or somatic splicing mutations detected in DNA or RNA sequence data; the level of expression of the proteasome, immunoproteasome, thymoproteasome, or other proteases in the infected cells (which may be measured by RNA-seq, proteome mass spectrometry, or immunohistochemistry); the expression of the source gene of the antigen encoded peptide (e.g., as measured by RNA-seq or mass spectrometry); the typical tissue-specific expression of the source gene of the antigen encoded peptide during various stages of the cell cycle; a comprehensive catalog of features of the source protein and/or its domains as can be found in e.g. uniProt or PDB http://www.rcsb.org/pdb/home/home.do; features describing the properties of the domain of the source protein containing the peptide, for example: secondary or tertiary structure (e.g., alpha helix vs beta sheet); alternative splicing; the probability of presentation of peptides from the source protein of the antigen encoded peptide in question in other distinct subjects; the probability that the peptide will not be detected or over-represented by mass spectrometry due to technical biases; the expression of various gene modules/pathways as measured by RNASeq (which need not contain the source protein of the peptide) that are informative about the state of the infected cells, stroma, or infected tissue; the copy number of the source gene of the antigen encoded peptide in the infected cells; the probability that the peptide binds to the TAP or the measured or predicted binding affinity of the peptide to the TAP; the expression level of TAP in the infected cells (which may be measured by RNA-seq, proteome mass spectrometry, immunohistochemistry). Peptides presentation may rely on a component of the antigen-presentation machinery that is subject to presence or absence of functional germline polymorphisms, including, but not limited to: in genes encoding the proteins involved in the antigen presentation machinery (e.g., B2M, HLA-A, HLA-B, HLA-C, TAP-1, TAP-2, TAPBP, CALR, CNX, ERP57, HLA-DM, HLA-DMA, HLA-DMB, HLA-DO, HLA-DOA, HLA-DOB, HLA-DP, HLA-DPA1, HLA-DPB1, HLA-DQ, HLA-DQA1, HLA-DQA2, HLA-DQB1, HLA-DQB2, HLA-DR, HLA-DRA, HLA-DRB1, HLA-DRB3, HLA-DRB4, HLA-DRB5 or any of the genes coding for components of the proteasome or immunoproteasome); infection type (e.g., a pathogen infection, a viral infection, a bacterial infection, an fungal infection, and a parasitic infection); clinical infection subtype (e.g., an HIV infection, a Severe acute respiratory syndrome-related coronavirus (SARS) infection, a severe acute respiratory syndrome coronavirus 2 (SARS-CoV-2) infection, a Ebola infection, a Hepatitis B virus (HBV) infection, an influenza infection, and a Hepatitis C virus (HCV) infection); the typical expression of the source gene of the peptide in the relevant infection type or clinical subtype.

A method disclosed herein may also include obtaining an infectious disease vaccine comprising the set of selected antigens (e.g., infectious disease organism derived antigens) or a subset thereof, optionally further comprising administering the infectious disease vaccine to the subject.

At least one of the antigens (e.g., infectious disease organism derived antigens) in the set of selected antigens, when in polypeptide form, may include at least one of: a binding affinity with MHC with an IC50 value of less than 1000 nM, for MHC Class I polypeptides a length of 8-15, 8, 9, 10, 11, 12, 13, 14, or 15 amino acids, for MHC Class II polypeptides a length of 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the polypeptide in the parent protein sequence promoting proteasome cleavage, and presence of sequence motifs promoting TAP transport. For MHC Class II, presence of sequence motifs within or near the peptide promoting cleavage by extracellular or lysosomal proteases (e.g., cathepsins) or HLA-DM catalyzed HLA binding.

Disclosed herein is are methods for identifying one or more antigens (e.g., infectious disease organism derived antigens) that are likely to be presented on a cell surface of an infected cell, comprising executing the steps of: receiving mass spectrometry data comprising data associated with a plurality of isolated peptides eluted from major histocompatibility complex (MHC) derived from a plurality of fresh or frozen samples; obtaining a training data set by at least identifying a set of training peptide sequences present in the samples and presented on one or more MHC alleles associated with each training peptide sequence; obtaining a set of training protein sequences based on the training peptide sequences; and training a set of numerical parameters of a presentation model using the training protein sequences and the training peptide sequences, the presentation model providing a plurality of numerical likelihoods that peptide sequences from the infected cell are presented by one or more MHC alleles on the infected cell surface.

The presentation model may represent dependence between: presence of a pair of a particular one of the MHC alleles and a particular amino acid at a particular position of a peptide sequence; and likelihood of presentation on the infected cell surface, by the particular one of the MHC alleles of the pair, of such a peptide sequence comprising the particular amino acid at the particular position.

A method disclosed herein can also include selecting a subset of antigens (e.g., infectious disease organism derived antigens), wherein the subset of antigens is selected because each has an increased likelihood that it is presented on the cell surface of the infected cell relative to one or more distinct antigens.

A method disclosed herein can also include selecting a subset of antigens (e.g., infectious disease organism derived antigens), wherein the subset of antigens is selected because each has an increased likelihood that it is capable of inducing a disease-specific immune response in the subject relative to one or more distinct antigens.

A method disclosed herein can also include selecting a subset of antigens (e.g., infectious disease organism derived antigens), wherein the subset of antigens is selected because each has an increased likelihood that it is capable of being presented to naïve T cells by professional antigen presenting cells (APCs) relative to one or more distinct antigens, optionally wherein the APC is a dendritic cell (DC).

A method disclosed herein can also include selecting a subset of antigens (e.g., infectious disease organism derived antigens), wherein the subset of antigens is selected because each has a decreased likelihood that it is subject to inhibition via central or peripheral tolerance relative to one or more distinct antigens.

A method disclosed herein can also include selecting a subset of antigens (e.g., infectious disease organism derived antigens), wherein the subset of antigens is selected because each has a decreased likelihood that it is capable of inducing an autoimmune response to normal tissue in the subject relative to one or more distinct antigens.

A method disclosed herein can also include selecting a subset of antigens (e.g., infectious disease organism derived antigens), wherein the subset of antigens is selected because each has a decreased likelihood that it will be differentially post-translationally modified in infected cells versus APCs, optionally wherein the APC is a dendritic cell (DC).

The practice of the methods herein will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B(1992).

### IV. Antigens

Antigens can include nucleotides or polypeptides. For example, an antigen can be an RNA sequence that encodes for a polypeptide sequence. Antigens useful in vaccines can therefore include nucleotide sequences or polypeptide sequences. Antigens can be derived from nucleotide sequences or polypeptide sequences of an infectious disease organism. Polypeptide sequences of an infectious disease organism include, but are not limited to, a pathogen-derived peptide, a virus-derived peptide, a bacteria-derived peptide, a fungus-derived peptide, and/or a parasite-derived peptide. Infectious disease organism include, but are not limited to, severe acute respiratory syndrome-related coronavirus (SARS), severe acute respiratory syndrome coronavirus 2 (SARS-CoV-2), Ebola, HIV, Hepatitis B virus (HBV), influenza, Hepatitis C virus (HCV), Human papillomavirus (HPV), Cytomegalovirus (CMV), Chikungunya virus, Respiratory syncytial virus (RSV), Dengue virus, a orthymyxoviridae family virus, tuberculosis, pancorona, herpes simplex virus infection (HSV), flu, metapneumovirus (MPV), and Parainfluenza Viruses (PIVs).

Disclosed herein are isolated peptides that comprise infectious disease organism specific antigens or epitopes identified by the methods disclosed herein, peptides that comprise known infectious disease organism specific antigens or epitopes, and mutant polypeptides or fragments thereof identified by methods disclosed herein. Antigen peptides can be described in the context of their coding sequence where an antigen includes the nucleotide sequence (e.g., DNA or RNA) that codes for the related polypeptide sequence.

One or more polypeptides encoded by an antigen nucleotide sequence can comprise at least one of: a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more antigens can be presented on the surface of an infected cell.

One or more antigens can be is immunogenic in a subject having an infection, e.g., capable of eliciting a T cell response or a B cell response in the subject.

One or more antigens that induce an autoimmune response in a subject can be excluded from consideration in the context of vaccine generation for a subject having an infection.

The size of at least one antigenic peptide molecule can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the antigenic peptide molecules are equal to or less than 50 amino acids.

Antigenic peptides and polypeptides can be: for MHC Class 115 residues or less in length and usually consist of between about 8 and about 11 residues, particularly 9 or 10 residues; for MHC Class II, 6-30 residues, inclusive.

If desirable, a longer peptide can be designed in several ways. In one case, when presentation likelihoods of peptides on HLA alleles are predicted or known, a longer peptide could consist of either: (1) individual presented peptides with an extensions of 2-5 amino acids toward the N- and C-terminus of each corresponding gene product; (2) a concatenation of some or all of the presented peptides with extended sequences for each. In another case, when sequencing reveals a long (>10 residues) epitope sequence present in an infected cell, a longer peptide would consist of: (3) the entire stretch of epitope sequence present in an infected cell—thus bypassing the need for computational or in vitro test-based selection of the strongest HLA-presented shorter peptide. In both cases, use of a longer peptide allows endogenous processing by patient cells and may lead to more effective antigen presentation and induction of T cell responses. Longer peptides can also a full-length protein, a protein subunit, a protein domain, and combinations thereof of a peptide expressed in an infectious disease organism.

Antigenic peptides and polypeptides can be presented on an HLA protein. In some aspects antigenic peptides and polypeptides are presented on an HLA protein with greater affinity than a wild-type peptide. In some aspects, an antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not induce an autoimmune response and/or invoke immunological tolerance when administered to a subject.

Also provided are compositions comprising at least two or more antigenic peptides. In some embodiments the composition contains at least two distinct peptides. At least two distinct peptides can be derived from the same polypeptide. By distinct polypeptides is meant that the peptide vary by length, amino acid sequence, or both. The peptides are derived from any polypeptide known to or have been found to be expressed in an infectious disease organism.

Antigenic peptides and polypeptides having a desired activity or property can be modified to provide certain desired attributes, e.g., improved pharmacological characteristics, while increasing or at least retaining substantially all of the biological activity of the unmodified peptide to bind the desired MHC molecule and activate the appropriate T cell. For instance, antigenic peptide and polypeptides can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

Modifications of peptides and polypeptides with various amino acid mimetics or unnatural amino acids can be particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides can be conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4 degrees C.) for 15 minutes and then spun to pellet the precipitated serum proteins. The presence of the peptides is then determined by reversed-phase HPLC using stability-specific chromatography conditions.

The peptides and polypeptides can be modified to provide desired attributes other than improved serum half-life. For instance, the ability of the peptides to induce CTL activity can be enhanced by linkage to a sequence which contains at least one epitope that is capable of inducing a T helper cell response. Immunogenic peptides/T helper conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the peptide can be linked to the T helper peptide without a spacer.

An antigenic peptide can be linked to the T helper peptide either directly or via a spacer either at the amino or carboxy terminus of the peptide. The amino terminus of either the antigenic peptide or the T helper peptide can be acylated. Exemplary T helper peptides include tetanus toxoid 830-843, influenza 307-319, malaria circumsporozoite 382-398 and 378-389.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In a further aspect an antigen includes a nucleic acid (e.g. polynucleotide) that encodes an antigenic peptide or portion thereof. The polynucleotide can be, e.g., DNA, cDNA, PNA, CNA, RNA (e.g., mRNA), either single- and/or double-stranded, or native or stabilized forms of polynucleotides, such as, e.g., polynucleotides with a phosphorothiate backbone, or combinations thereof and it may or may not contain introns. A still further aspect provides an expression vector capable of expressing a polypeptide or portion thereof. Expression vectors for different cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found e.g. in Sambrook et al. (1989) Molecular Cloning, A Laboratory Manual, Cold Spring Harbor Laboratory, Cold Spring Harbor, N.Y.

### IV. Example Therapeutics

As disclosed herein, selected infectious disease-derived antigens (e.g., selected by predicting likelihood of presentation of candidate antigens) can be used to develop a therapeutic that, if administered in a patient, can elicit an immune response to an infectious disease. Example therapeutics include a vaccine composition, a composition comprising a T-cell receptor (TCR) or a chimeric antigen receptor (CAR) that binds to one or more selected infectious disease-derived antigens, and antibodies that exhibit binding specificity for one or more selected infectious disease-derived antigens. Further details of example therapeutics are described herein.

### IV.A. Vaccine Compositions

Also disclosed herein is an immunogenic composition, e.g., a vaccine composition, capable of raising a specific immune response, e.g., an immune response to an infectious disease. Vaccine compositions typically comprise a plurality of infectious disease-derived antigens, e.g., selected using a method described herein. Vaccine compositions can also be referred to as vaccines.

A vaccine can contain between 1 and 30 peptides, 2, 3, 4, 5, 6, 7, 8, 9, 10, 11, 12, 13, 14, 15, 16, 17, 18, 19, 20, 21, 22, 23, 24, 25, 26, 27, 28, 29, or 30 different peptides, 6, 7, 8, 9, 10 11, 12, 13, or 14 different peptides, or 12, 13 or 14 different peptides. A vaccine can contain between 1 and 100 or more nucleotide sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different nucleotide sequences, 6, 7, 8, 9, 10 11, 12, 13, or 14 different nucleotide sequences, or 12, 13 or 14 different nucleotide sequences. A vaccine can contain between 1 and 30 antigen sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen sequences, 6, 7, 8, 9, 10 11, 12, 13, or 14 different antigen sequences, or 12, 13 or 14 different antigen sequences.

In one embodiment, different peptides and/or polypeptides or nucleotide sequences encoding them are selected so that the peptides and/or polypeptides capable of associating with different MHC molecules, such as different MHC class I molecule. In some aspects, one vaccine composition comprises coding sequence for peptides and/or polypeptides capable of associating with the most frequently occurring MHC class I molecules. Hence, vaccine compositions can comprise different fragments capable of associating with at least 2 preferred, at least 3 preferred, or at least 4 preferred MHC class I molecules.

The vaccine composition can be capable of raising a specific cytotoxic T-cells response and/or a specific helper T-cell response.

A vaccine composition can further comprise an adjuvant and/or a carrier. Examples of useful adjuvants and carriers are given herein below. A composition can be associated with a carrier such as e.g. a protein or an antigen-presenting cell such as e.g. a dendritic cell (DC) capable of presenting the peptide to a T-cell.

Adjuvants are any substance whose admixture into a vaccine composition increases or otherwise modifies the immune response to an antigen. Carriers can be scaffold structures, for example a polypeptide or a polysaccharide, to which an antigen, is capable of being associated. Optionally, adjuvants are conjugated covalently or non-covalently.

The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, alum, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

A vaccine composition can comprise more than one different adjuvant. Furthermore, a therapeutic composition can comprise any adjuvant substance including any of the above or combinations thereof. It is also contemplated that a vaccine and an adjuvant can be administered together or separately in any appropriate sequence.

A carrier (or excipient) can be present independently of an adjuvant. The function of a carrier can for example be to increase the molecular weight of in particular mutant to increase activity or immunogenicity, to confer stability, to increase the biological activity, or to increase serum half-life. Furthermore, a carrier can aid presenting peptides to T-cells. A carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diptheria toxoid are suitable carriers. Alternatively, the carrier can be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments a vaccine composition additionally contains at least one antigen presenting cell.

Infectious disease-derived antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. A wide variety of other vaccine vectors useful for therapeutic administration or immunization of antigens, e.g., Salmonella typhi vectors, and the like will be apparent to those skilled in the art from the description herein.

### IV.A.1. Considerations for Vaccine Design and Manufacture

Truncal peptides, meaning those presented by all or most tumor subclones, will be prioritized for inclusion into the vaccine. Optionally, if there are no truncal peptides predicted to be presented and immunogenic with high probability, or if the number of truncal peptides predicted to be presented and immunogenic with high probability is small enough that additional non-truncal peptides can be included in the vaccine, then further peptides can be prioritized by estimating the number and identity of tumor subclones and choosing peptides so as to maximize the number of tumor subclones covered by the vaccine.

Additional candidate antigens may still be available for vaccine inclusion than the vaccine technology can support. Additionally, uncertainty about various aspects of the antigen analysis may remain and tradeoffs may exist between different properties of candidate vaccine antigens. Thus, in place of predetermined filters at each step of the selection process, an integrated multi-dimensional model can be considered that places candidate antigens in a space with at least the following axes and optimizes selection using an integrative approach.
1. Risk of auto-immunity or tolerance (risk of germline) (lower risk of auto-immunity is typically preferred)
2. Probability of sequencing artifact (lower probability of artifact is typically preferred)
3. Probability of immunogenicity (higher probability of immunogenicity is typically preferred)
4. Probability of presentation (higher probability of presentation is typically preferred)
5. Gene expression (higher expression is typically preferred)
6. Coverage of HLA genes (larger number of HLA molecules involved in the presentation of a set of antigens may lower the probability that a tumor will escape immune attack via downregulation or mutation of HLA molecules)

In various embodiments, vaccine design involves the steps of 1) identifying a set of epitopes, 2) aligning the epitopes to a reference proteome to define an initial footprint, 3) ranking remaining epitopes according to coverage and cost to cassette size, 4) including top ranked epitope, and 5) repeating steps (3) and (4) until maximum cassette design is reached or all epitopes are included.

Referring to step (1), identifying a set of epitopes may include a set of short amino-acid sequences. In various embodiments, the short amino-acid sequences have lengths between 8 and 12 amino acids. In various embodiments, the short amino-acid sequences have lengths between 9 and 11 amino acids, In various embodiments, the short amino-acid sequences have lengths of 8 amino acids, 9 amino acids, 10 amino acids, 11 amino acids, or 12 amino acids. In various embodiments, the set of epitopes include epitopes identified as likely to be presented by MHC alleles using methods disclosed herein (e.g., using a presentation model disclosed herein). In various embodiments, the set of epitopes include epitopes that are publicly documented as likely to be presented by MHC alleles. Example epitopes that are publicly documents as likely to be presented can be found in the Immune Epitope Database (IEDB) and/or the Los Alamos National Laboratory HIV CD8 T-cell epitope database. The Immune Epitope Database (IEDB) is described in further detail in Vita R, et al, The Immune Epitope Database (IEDB): 2018 update. Nucleic Acids Res. 2018 Oct. 24, which is incorporated by reference in its entirety. The Los Alamos National Laboratory HIV CD8 T-cell epitope database is described in further detail in Llano, A., et al., The 2019 Optimal HIV CTL epitopes update: Growing diversity in epitope length and HLA restriction. HIV Molecular Immunology 2019, 3-27, which is incorporated by reference in its entirety.

In various embodiments, the set of epitopes include both 1) epitopes identified as likely to be presented by MHC alleles using methods disclosed herein (e.g., using a presentation model disclosed herein) and 2) epitopes that are publicly documented as likely to be presented by MHC alleles. In particular embodiments, the set of validated epitopes include epitopes identified as likely to be presented by class I MHC alleles.

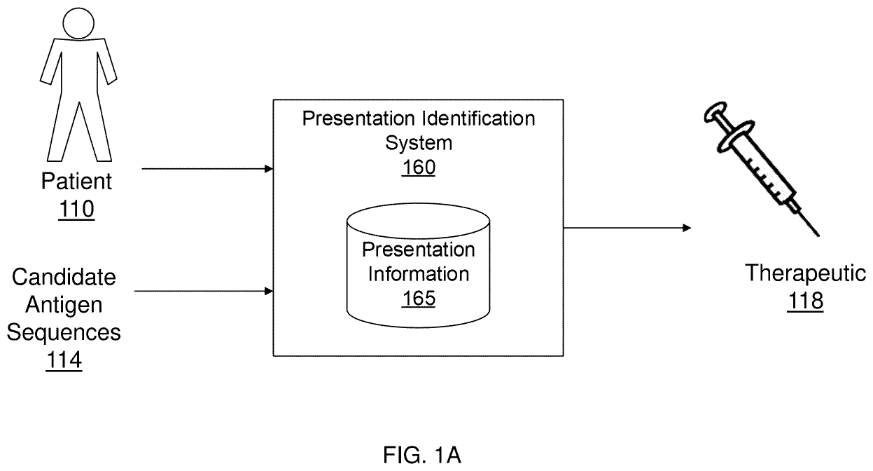

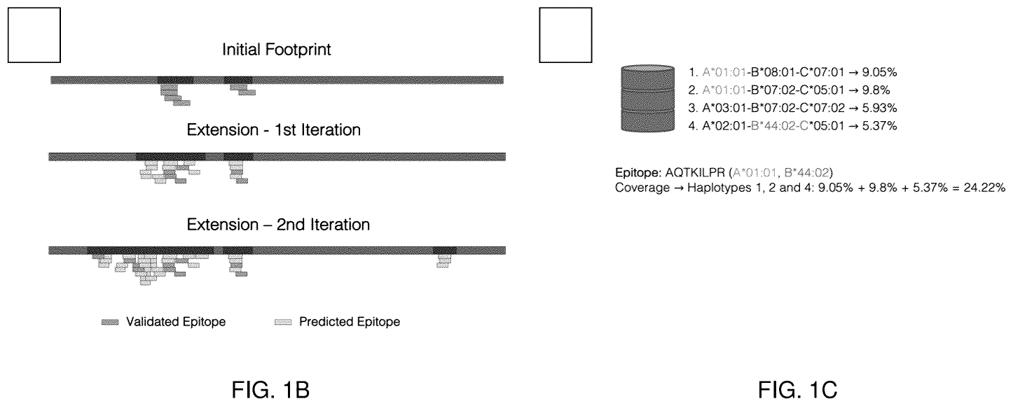

Step (2) involves aligning one or more epitopes of the set of epitopes to a reference proteome. Reference is made to FIG. 1B, which shows an example cassette design methodology, in accordance with an embodiment. Here, the one or more epitopes are aligned to the reference proteome to generate an initial footprint. In various embodiments, the one or more epitopes are validated epitopes (e.g., epitopes that are publicly documented as likely to be presented by MHC alleles). In various embodiments, the initial footprint is constrained using two or more parameters that control for the size of the initial footprint. For example parameters can include a minimum epitope length (min_{length}), a minimum required number of overlapping (minₒᵥₑᵣₗₐₚ) epitopes, and/or a proportion (minₚᵣₒₚ) of the minimum epitope length (min_{length}) that have a minimum required number of overlapping (minₒᵥₑᵣₗₐₚ) epitopes. In various embodiments, the minimum epitope length (min_{length}) is at least 2 amino acids. In various embodiments, the minimum epitope length is at least 3 amino acids, at least 4 amino acids, at least 5 amino acids, at least 6 amino acids, at least 7 amino acids, at least 8 amino acids, at least 9 amino acids, or at least 10 amino acids. In various embodiments, the minimum required number of overlapping (minₒᵥₑᵣₗₐₚ) epitopes is at least 2 overlapping epitopes. In various embodiments, the minimum required number of overlapping (minₒᵥₑᵣₗₐₚ) epitopes is at least 3 overlapping epitopes, at least 4 overlapping epitopes, at least 5 overlapping epitopes, at least 6 overlapping epitopes, at least 7 overlapping epitopes, at least 8 overlapping epitopes, at least 9 overlapping epitopes, or at least 10 overlapping epitopes.

Step (3) involves ranking remaining epitopes according to coverage and cost to cassette size. Specifically, coverage is denoted as c and refers to the added population coverage provided by the epitope if it were included in the cassette. In various embodiments, the coverage provided by a given epitope is calculated according to frequencies of haplotypes in a reference population that cover at least one allele associated with the epitope. Example reference populations can include any of the ancestry groups African-American (AFA), Hispanic (HIS), Asian-Pacific Islanders (API) and Europeans (EUR). Example reference population data can be found in the US National Marrow Donor Program (e.g., Bioinformatics Be The Match®). Reference is made to FIG. 1C, which shows an example epitope sequence (AQTKILPR) along with example alleles. Here, coverage is calculated with respect to two of the alleles (e.g., A*01:01 and B*08:01). The total coverage represents the sum of the haplotypes containing either or both of the two alleles (e.g., A*01:01 and B*08:01). Cost to cassette size is denoted as ƒ and refers to the total increase in cassette size as a consequence of addition of the epitope. In various embodiments, the epitopes are ranked according to the coverage and cost to cassette size such that the best ranked epitopes provide the best improvement in coverage at a least cost to cassette size.

Step (4) involves including a top ranked epitope in the cassette design. For example, as shown in FIG. 1B under “Extension—1^{st} iteration”, additional epitopes, such as the top ranked epitopes, are added to expand upon the initial footprint.

Step (5) involves further repeating the ranking step (3) amongst the remaining epitopes of the set and further repeating step (4) of including the top ranked epitope in the cassette design. Referring agains tot FIG. 1B under “Extension—2^{nd} iteration”, yet additional epitopes, such as top ranked epitopes identified during this iteration, can be included to further expand upon the footprint. Steps (3) and (4) can continue to be repeated until a maximum cassette design is reached or until all epitopes are included in the cassette.

### IV.B. T Cell Receptors (TCRs) and/or Chimerican Antigen Receptors (CARs)

Additionally disclosed herein are T cell receptors (TCRs) that are designed to bind to antigens predicted to be presented on the surface of cells. The TCRs may be isolated and purified. In a majority of T-cells, the TCR is a heterodimer polypeptide having an alpha (α) chain and beta- (β) chain, encoded by TRA and TRB, respectively. The alpha chain generally comprises an alpha variable region, encoded by TRAV, an alpha joining region, encoded by TRAJ, and an alpha constant region, encoded by TRAC. The beta chain generally comprises a beta variable region, encoded by TRBV, a beta diversity region, encoded by TRBD, a beta joining region, encoded by TRBJ, and a beta constant region, encoded by TRBC. The TCR-alpha chain is generated by VJ recombination of alpha V and J segments, and the beta chain receptor is generated by V(D)J recombination of beta V, D, and J segments. Additional TCR diversity stems from junctional diversity. Several bases may be deleted and others added (called N and P nucleotides) at each of the junctions. In a minority of T-cells, the TCRs include gamma and delta chains. The TCR gamma chain is generated by VJ recombination, and the TCR delta chain is generated by V(D)J recombination (Kenneth Murphy, Paul Travers, and Mark Walport, Janeway's Immunology 7th edition, Garland Science, 2007, which is herein incorporated by reference in its entirety). The antigen binding site of a TCR generally comprises six complementarity determining regions (CDRs). The alpha chain contributes three CDRs, alpha (“α”) CDR1, αCDR2, and αCDR3. The beta chain also contributes three CDR: beta (“β”) CDR1, βCDR2, and βCDR3. In general, the αCDR3 and βCDR3 are the regions most affected by V(D)J recombination and account for most of the variation in a TCR repertoire.

TCRs can be designed to specifically recognize antigens disclosed herein, such as infectious disease-derived antigens that are predicted to be presented on the surface of cells. In various embodiments, TCRs can also be membrane-bound, e.g., on a cell such as a T cell or natural killer (NK) cell. Thus, TCRs can be used in a context that corresponds to soluble antibodies and/or membrane-bound CARs.

Any of the TCRs disclosed herein may comprise an alpha variable (“V”) segment, an alpha joining (“J”) segment, optionally an alpha constant region, a beta variable (“V”) segment, optionally a beta diversity (“D”) segment, a beta joining (“J”) segment, and optionally a beta constant region.

In some embodiments, the TCR or CAR is a recombinant TCR or CAR. The recombinant TCR or CAR may include any of the TCRs identified herein but include one or more modifications. Exemplary modifications, e.g., amino acid substitutions, are described herein. Amino acid substitutions described herein may be made with reference to IMGT nomenclature and amino acid numbering as found at www.imgt.org.

The recombinant TCR or CAR may be a human TCR or CAR, comprising fully human sequences, e.g., natural human sequences. The recombinant TCR or CAR may retain its natural human variable domain sequences but contain modifications to the α constant region, β constant region, or both α and β constant regions. Such modifications to the TCR constant regions may improve TCR assembly and expression for TCR gene therapy by, e.g., driving preferential pairings of the exogenous TCR chains.

In some embodiments, the α and β constant regions are modified by substituting the entire human constant region sequences for mouse constant region sequences. Such “murinized” TCRs and methods of making them are described in Cancer Res. 2006 Sep. 1; 66(17):8878-86, which is hereby incorporated by reference in its entirety.

In some embodiments, the α and β constant regions are modified by making one or more amino acid substitutions in the human TCR α constant (TRAC) region, the TCR R constant (TRBC) region, or the TRAC and TRAB regions, which swap particular human residues for murine residues (human→murine amino acid exchange). The one or more amino acid substitutions in the TRAC region may include a Ser substitution at residue 90, an Asp substitution at residue 91, a Val substitution at residue 92, a Pro substitution at residue 93, or any combination thereof. The one or more amino acid substitutions in the human TRBC region may include a Lys substitution at residue 18, an Ala substitution at residue 22, an Ile substitution at residue 133, a His substitution at residue 139, or any combination of the above. Such targeted amino acid substitutions are described in J Immunol Jun. 1, 2010, 184 (11) 6223-6231, which is hereby incorporated by reference in its entirety.

In some embodiments, the human TRAC contains an Asp substitution at residue 210 and the human TRBC contains a Lys substitution at residue 134. Such substitutions may promote the formation of a salt bridge between the alpha and beta chains and formation of the TCR interchain disulfide bond. These targeted substitutions are described in J Immunol Jun. 1, 2010, 184 (11) 6232-6241, which is hereby incorporated by reference in its entirety.

In some embodiments, the human TRAC and human TRBC regions are modified to contain introduced cysteines which may improve preferential pairing of the exogenous TCR chains through formation of an additional disulfide bond. For example, the human TRAC may contain a Cys substitution at residue 48 and the human TRBC may contain a Cys substitution at residue 57, described in Cancer Res. 2007 Apr. 15; 67(8):3898-903 and Blood. 2007 Mar. 15; 109(6):2331-8, which are hereby incorporated by reference in their entirety.

The recombinant TCR or CAR may comprise other modifications to the α and β chains.

In some embodiments, the α and β chains are modified by linking the extracellular domains of the α and β chains to a complete human CD3δ((CD3-zeta) molecule. Such modifications are described in J Immunol Jun. 1, 2008, 180 (11) 7736-7746; Gene Ther. 2000 August; 7(16):1369-77; and The Open Gene Therapy Journal, 2011, 4: 11-22, which are hereby incorporated by reference in their entirety.

In some embodiments, the α chain is modified by introducing hydrophobic amino acid substitutions in the transmembrane region of the α chain, as described in J Immunol Jun. 1, 2012, 188 (11) 5538-5546; hereby incorporated by reference in their entirety.

The alpha or beta chain may be modified by altering any one of the N-glycosylation sites in the amino acid sequence, as described in J Exp Med. 2009 Feb. 16; 206(2): 463-475; hereby incorporated by reference in its entirety.

The alpha and beta chain may each comprise a dimerization domain, e.g., a heterologous dimerization domain. Such a heterologous domain may be a leucine zipper, a 5H3 domain or hydrophobic proline rich counter domains, or other similar modalities, as known in the art. In one example, the alpha and beta chains may be modified by introducing 30mer segments to the carboxyl termini of the alpha and beta extracellular domains, wherein the segments selectively associate to form a stable leucine zipper. Such modifications are described in PNAS Nov. 22, 1994. 91 (24) 11408-11412; https://doi.org/10.1073/pnas.91.24.11408; hereby incorporated by reference in its entirety.

TCRs identified herein may be modified to include mutations that result in increased affinity or half-life, such as those described in WO2012/013913, hereby incorporated by reference in its entirety.

The recombinant TCR or CAR may be a single chain TCR (scTCR). Such scTCR may comprise an a chain variable region sequence fused to the N terminus of a TCR α chain constant region extracellular sequence, a TCR β chain variable region fused to the N terminus of a TCR β chain constant region extracellular sequence, and a linker sequence linking the C terminus of the α segment to the N terminus of the β segment, or vice versa. In some embodiments, the constant region extracellular sequences of the α and β segments of the scTCR are linked by a disulfide bond. In some embodiments, the length of the linker sequence and the position of the disulfide bond being such that the variable region sequences of the α and β segments are mutually orientated substantially as in native αβ T cell receptors. Exemplary scTCRs are described in U.S. Pat. No. 7,569,664, which is hereby incorporated by reference in its entirety.

In some cases, the variable regions of the scTCR may be covalently joined by a short peptide linker, such as described in Gene Therapy volume 7, pages 1369-1377 (2000). The short peptide linker may be a serine rich or glycine rich linker. For example, the linker may be (Gly₄Ser)₃, as described in Cancer Gene Therapy (2004) 11, 487-496, incorporated by reference in its entirety.

The recombinant TCR or antigen binding fragment thereof may be expressed as a fusion protein. For instance, the TCR or antigen binding fragment thereof may be fused with a toxin. Such fusion proteins are described in Cancer Res. 2002 Mar. 15; 62(6):1757-60. The TCR or antigen binding fragment thereof may be fused with an antibody Fc region. Such fusion proteins are described in J Immunol May 1, 2017, 198 (1 Supplement) 120.9.

The antigen recognition domain of a receptor such as a TCR or CAR can be linked to one or more intracellular signaling components, such as signaling components that mimic activation through an antigen receptor complex, such as a TCR complex and/or signal via another cell surface receptor. For example, the TCR or CAR can be linked to one or more transmembrane and/or intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, and/or CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the receptor.

The receptor, e.g., the TCR or CAR, can include at least one intracellular signaling component or components. In some embodiments, the receptor includes an intracellular component of a TCR complex, such as a TCR CD3 chain that mediates T-cell activation and cytotoxicity, e.g., CD3 zeta chain. For example, the HLA-PEPTIDE-binding ABP (e.g., a TCR or CAR) is linked to one or more cell signaling modules. In some embodiments, cell signaling modules include CD3 transmembrane domain, CD3 intracellular signaling domains, and/or other CD transmembrane domains. In some embodiments, the receptor, e.g., a TCR or CAR, further includes a portion of one or more additional molecules such as Fc receptor-gamma, CD8, CD4, CD25, or CD16. For example, in some aspects, the TCR or CAR includes a chimeric molecule between CD3-zeta or Fc receptor-gamma and CD8, CD4, CD25 or CD16.

In some embodiments, upon ligation of the TCR or CAR, the cytoplasmic domain or intracellular signaling domain of the receptor activates at least one of the normal effector functions or responses of the immune cell, e.g., T cell engineered to express the receptor. For example, in some contexts, the receptor induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the receptor. In other embodiments, the receptor does not include a component for generating a costimulatory signal. In some aspects, an additional receptor is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the receptor includes one or both of such signaling components.

In some aspects, the receptor includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

In some embodiments, the receptor includes a signaling domain and/or transmembrane portion of a costimulatory receptor, such as CD28, 4-1BB, OX40, DAP10, and ICOS. In some aspects, the same receptor includes both the activating and costimulatory components.

In some embodiments, the activating domain is included within one receptor, whereas the costimulatory component is provided by another receptor recognizing another antigen. In some embodiments, the receptors include activating or stimulatory receptors, and costimulatory receptors, both expressed on the same cell (see WO2014/055668). In some aspects, the HLA-PEPTIDE-targeting receptor is the stimulatory or activating receptor; in other aspects, it is the costimulatory receptor. In some embodiments, the cells further include inhibitory receptors (e.g., iCARs, see Fedorov et al., Sci. Transl. Medicine, 5(215) (December, 2013), such as a receptor recognizing an antigen other than HLA-PEPTIDE, whereby an activating signal delivered through the HLA-PEPTIDE-targeting receptor is diminished or inhibited by binding of the inhibitory receptor to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the receptor encompasses one or more, e.g., two or more, costimulatory domains and an activation domain, e.g., primary activation domain, in the cytoplasmic portion. Exemplary receptors include intracellular components of CD3-zeta, CD28, and 4-1BB.

In some embodiments, the CAR (or other antigen receptor such as a TCR) further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a nerve growth factor receptor (NGFR), or epidermal growth factor receptor (e.g., tEGFR). In some embodiments, the nucleic acid encoding the marker is operably linked to a polynucleotide encoding for a linker sequence, such as a cleavable linker sequence or a ribosomal skip sequence, e.g., T2A. See WO2014031687. In some embodiments, introduction of a construct encoding the CAR and EGFRt separated by a T2A ribosome switch can express two proteins from the same construct, such that the EGFRt can be used as a marker to detect cells expressing such construct. In some embodiments, a marker, and optionally a linker sequence, can be any as disclosed in published patent application No. WO2014031687. For example, the marker can be a truncated EGFR (tEGFR) that is, optionally, linked to a linker sequence, such as a T2A ribosomal skip sequence.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

The TCR or CAR may comprise one or modified synthetic amino acids in place of one or more naturally-occurring amino acids. Exemplary modified amino acids include, but are not limited to, aminocyclohexane carboxylic acid, norleucine, α-amino n-decanoic acid, homoserine, S-acetylaminomethylcysteine, trans-3- and trans-4-hydroxyproline, 4-aminophenylalanine, 4-nitrophenylalanine, 4-chlorophenylalanine, 4-carboxyphenylalanine, (3-phenylserine (3-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N‘-benzyl-N’-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,γ-diaminopropionic acid, homophenylalanine, and α-tertbutylglycine.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular portion containing a TCR or fragment described herein. In some aspects, the chimeric antigen receptor includes an extracellular portion containing a TCR or fragment described herein and an intracellular signaling domain. In some embodiments, the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain linking the extracellular domain and the intracellular signaling domain.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 41BB.

In some embodiments, the CAR contains a TCR, e.g., a TCR fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains a TCR, e.g., a TCR fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the TCR or CAR, is a transmembrane domain of human CD28 or variant thereof, e.g., a 27-amino acid transmembrane domain of a human CD28 (Accession No.: P10747.1).

In some embodiments, the chimeric antigen receptor contains an intracellular domain of a T cell costimulatory molecule. In some aspects, the T cell costimulatory molecule is CD28 or 41BB.

In some embodiments, the intracellular signaling domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular domain comprises an intracellular costimulatory signaling domain of 41BB or functional variant or portion thereof, such as a 42-amino acid cytoplasmic domain of a human 4-1BB (Accession No. Q07011.1) or functional variant or portion thereof.

In some embodiments, the intracellular signaling domain comprises a human CD3 zeta stimulatory signaling domain or functional variant thereof, such as a 112 AA cytoplasmic domain of isoform 3 of human CD3.zeta. (Accession No.: P20963.2) or a CD3 zeta signaling domain as described in U.S. Pat. No. 7,446,190 or U.S. Pat. No. 8,911,993.

In some aspects, the spacer contains only a hinge region of an IgG, such as only a hinge of IgG4 or IgG1. In other embodiments, the spacer is an Ig hinge, e.g., and IgG4 hinge, linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only. In some embodiments, the spacer is or comprises a glycine-serine rich sequence or other flexible linker such as known flexible linkers.

For example, in some embodiments, the CAR includes a TCR or fragment thereof, such as any of the HLA-PEPTIDE specific TCRs, a spacer such as any of the Ig-hinge containing spacers, a CD28 transmembrane domain, a CD28 intracellular signaling domain, and a CD3 zeta signaling domain. In some embodiments, the CAR includes a TCR or fragment, such as any of the HLA-PEPTIDE specific TCRs, a spacer such as any of the Ig-hinge containing spacers, a CD28 transmembrane domain, a CD28 intracellular signaling domain, and a CD3 zeta signaling domain.

### IV.C. Methods for Engineering Cells with TCRs and/or CARs

Also provided are methods, nucleic acids, compositions, and kits, for expressing receptors comprising TCRs, CARs, and the like, and for producing genetically engineered cells expressing such TCRs, CARs, and the like. The genetic engineering generally involves introduction of a nucleic acid encoding the recombinant or engineered component into the cell, such as by retroviral transduction, transfection, or transformation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell II: 223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells are further engineered to promote expression of cytokines or other factors. Various methods for the introduction of genetically engineered components, such as antigen receptors (e.g., TCRs), are well known and may be used with the provided methods and compositions. Exemplary methods include those for transfer of nucleic acids encoding the receptors, including via viral, e.g., retroviral or lentiviral transduction, transposons, nuclease mediated gene-editing (e.g., CRISPR, TALEN, meganuclease, or ZFN editing systems), and electroporation. For example, nuclease mediated gene-editing, particularly for editing T cells, is described in more detail in international applications WO/2018/232356 and PCT/US2018/058230, herein incorporated by reference for all purposes.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298; Van Tedeloo et al. (2000) Gene Therapy 7(16): 1431-1437; and Roth et al. (2018) Nature 559:405-409). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### IV.D. Antibody or Antigen-Binding Fragments

Additionally disclosed herein are antibodies or antigen-binding fragments that are designed to bind to antigens predicted to be presented on the surface of cells. In some embodiments, the antibodies or antigen-binding fragments provided herein comprise a light chain. In some aspects, the light chain is a kappa light chain. In some aspects, the light chain is a lambda light chain.

In some embodiments, the antibodies or antigen-binding fragments provided herein comprise a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibodies or antigen-binding fragments provided herein comprise an antibody fragment. In some embodiments, the antibodies or antigen-binding fragments provided herein consist of an antibody fragment. In some embodiments, the antibodies or antigen-binding fragments provided herein consist essentially of an antibody fragment. In some aspects, the antibody fragment is an Fv fragment. In some aspects, the antibody fragment is a Fab fragment. In some aspects, the antibody fragment is a F(ab′)₂ fragment. In some aspects, the antibody fragment is a Fab′ fragment. In some aspects, the antibody fragment is an scFv (sFv) fragment. In some aspects, the antibody fragment is an scFv-Fc fragment. In some aspects, the antibody fragment is a fragment of a single domain ABP.

In some embodiments, an antibody fragment provided herein retains the ability to bind a target, such as an infectious disease-derived antigen predicted to be presented by one or more HLA alleles on a surface of a cell, as measured by one or more assays or biological effects described herein. In some embodiments, an antibody fragment provided herein retains the ability to prevent an infectious disease-derived antigen from interacting with one or more of its ligands, as described herein.

The antibody fragments provided herein may be made by any suitable method, including the illustrative methods described herein or those known in the art. Suitable methods include recombinant techniques and proteolytic digestion of whole antibodies or antigen-binding fragments.

In some embodiments, the antibodies provided herein are monoclonal antibodies. Monoclonal antibodies may be obtained, for example, using a hybridoma method or using phage or yeast-based libraries. DNA encoding the monoclonal antibodies may be readily isolated and sequenced using conventional procedures. In some embodiments, the antibodies provided herein are polyclonal antibodies. In some embodiments, the antibodies provided herein comprise a chimeric ABP. In some embodiments, the antibodies provided herein consist of a chimeric antibody. In some embodiments, the antibodies provided herein consist essentially of a chimeric antibody. Chimeric antibodies can be made by any methods known in the art. In some embodiments, a chimeric antibody is made by using recombinant techniques to combine a non-human variable region (e.g., a variable region derived from a mouse, rat, hamster, rabbit, or non-human primate, such as a monkey) with a human constant region.

In some embodiments, the antibodies provided herein comprise a humanized antibody. In some embodiments, the antibodies provided herein consist of a humanized antibody. In some embodiments, the antibodies provided herein consist essentially of a humanized antibody. Humanized antibodies may be generated by replacing most, or all, of the structural portions of a non-human monoclonal antibody with corresponding human antibody sequences.

In some embodiments, the antibodies provided herein comprise a human antibody. In some embodiments, the antibodies provided herein consist of a human antibody. In some embodiments, the antibodies provided herein consist essentially of a human antibody. Human antibodies can be generated by a variety of techniques known in the art, for example by using transgenic animals (e.g., humanized mice), can be derived from phage-display libraries, can be generated by in vitro activated B cells, or can be derived from yeast-based libraries

In some embodiments, the antibodies provided herein comprise an alternative scaffold. In some embodiments, the antibodies provided herein consist of an alternative scaffold. In some embodiments, the antibodies provided herein consist essentially of an alternative scaffold. Any suitable alternative scaffold may be used. In some aspects, the alternative scaffold is selected from an Adnectin™, an iMab, an Anticalin®, an EETI-II/AGRP, a Kunitz domain, a thioredoxin peptide aptamer, an Affibody®, a DARPin, an Affilin, a Tetranectin, a Fynomer, and an Avimer. The alternative scaffolds provided herein may be made by any suitable method, including the illustrative methods described herein or those known in the art.

Also disclosed herein is an isolated humanized, human, or chimeric antibody that competes for binding to a HLA-antigen complex with an antibody disclosed herein.

In certain aspects, an antibody may comprise a human Fc region comprising at least one modification that reduces binding to a human Fc receptor. It is known that when an antibody is expressed in cells, the antibody is modified after translation. Examples of the posttranslational modification include cleavage of lysine at the C terminus of the heavy chain by a carboxypeptidase; modification of glutamine or glutamic acid at the N terminus of the heavy chain and the light chain to pyroglutamic acid by pyroglutamylation; glycosylation; oxidation; deamidation; and glycation, and it is known that such posttranslational modifications occur in various ABPs (See Journal of Pharmaceutical Sciences, 2008, Vol. 97, p. 2426-2447, incorporated by reference in its entirety). In some embodiments, an antibody is an antibody or antigen-binding fragment thereof which has undergone posttranslational modification. Examples of an antibody or antigen-binding fragment thereof which have undergone posttranslational modification include an antibody or antigen-binding fragments thereof which have undergone pyroglutamylation at the N terminus of the heavy chain variable region and/or deletion of lysine at the C terminus of the heavy chain. It is known in the art that such posttranslational modification due to pyroglutamylation at the N terminus and deletion of lysine at the C terminus does not have any influence on the activity of the antibody or fragment thereof (Analytical Biochemistry, 2006, Vol. 348, p. 24-39, incorporated by reference in its entirety).

In some embodiments, the antibodies provided herein are multispecific antibodies.

In some embodiments, a multispecific antibody provided herein binds more than one antigen. In some embodiments, a multispecific antibody binds 2 antigens (e.g., a bispecific antibody). In some embodiments, a multispecific antibody binds 3 antigens. In some embodiments, a multispecific antibody binds 4 antigens. In some embodiments, a multispecific antibody binds 5 antigens.

In some embodiments, the multispecific ABP comprises an antigen-binding domain (ABD) that specifically binds to an HLA-PEPTIDE target disclosed herein and an additional ABD that binds to an additional target antigen. Many multispecific antibody constructs are known in the art, and the antibodies provided herein may be provided in the form of any suitable multispecific construct. The multispecific antibodies provided herein may be made by any suitable method, including the illustrative methods described herein or those known in the art.

In certain embodiments, an antibody provided herein comprises an Fc region. An Fc region can be wild-type or a variant thereof. In certain embodiments, an antibody provided herein comprises an Fc region with one or more amino acid substitutions, insertions, or deletions in comparison to a naturally occurring Fc region. In some aspects, such substitutions, insertions, or deletions yield an antibody with altered stability, glycosylation, or other characteristics. In some aspects, such substitutions, insertions, or deletions yield a glycosylated antibody.

In some embodiments, the Fc region is a variant Fc region. A “variant Fc region” or “engineered Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, an antibody having an Fc region can comprise an antibody with or without K447.

In some aspects, the Fc region of an antibody provided herein is modified to yield an antibody with altered affinity for an Fc receptor, or an antibody that is more immunologically inert. In some embodiments, the antibody variants provided herein possess some, but not all, effector functions. Such antibodies may be useful, for example, when the half-life of the antibody is important in vivo, but when certain effector functions (e.g., complement activation and ADCC) are unnecessary or deleterious.

In some embodiments, an antibody provided herein comprises one or more alterations that improves or diminishes C1q binding and/or CDC.

In some embodiments, an antibody provided herein comprises one or more alterations to increase half-life. In some embodiments, the antibody comprises one or more non-Fc modifications that extend half-life.

In some embodiments, the multispecific antibody comprises one or more Fc modifications that promote heteromultimerization. In some embodiments, the Fc modification comprises a set of mutations that renders homodimerization electrostatically unfavorable but heterodimerization favorable.

In some embodiments, the Fc modification comprises a modification in the CH3 sequence that affects the ability of the CH3 domain to bind an affinity agent, e.g., Protein A.

### V. Therapeutic and Manufacturing Methods

Also provided is a method of inducing an infectious disease organism-specific immune response in a subject, vaccinating against an infectious disease organism, treating and or alleviating a symptom of an infection associated with an infectious disease organism in a subject by administering to the subject one or more antigens such as a plurality of antigens identified using methods disclosed herein.

In some aspects, a subject has been diagnosed with an infection or is at risk of an infection. A subject can be a human, dog, cat, horse or any animal in which an infectious disease organism specific immune response is desired.

An antigen can be administered in an amount sufficient to induce a CTL response. An antigen can be administered in an amount sufficient to induce a T cell response. An antigen can be administered in an amount sufficient to induce a B cell response.

An antigen can be administered alone or in combination with other therapeutic agents. Any suitable therapeutic treatment for a particular infectious disease can be administered.

The optimum amount of each antigen to be included in a vaccine composition and the optimum dosing regimen can be determined. For example, an antigen or its variant can be prepared for intravenous (i.v.) injection, sub-cutaneous (s.c.) injection, intradermal (i.d.) injection, intraperitoneal (i.p.) injection, intramuscular (i.m.) injection. Methods of injection include s.c., i.d., i.p., i.m., and i.v. Methods of DNA or RNA injection include i.d., i.m., s.c., i.p. and i.v. Other methods of administration of the vaccine composition are known to those skilled in the art.

A vaccine can be compiled so that the selection, number and/or amount of antigens present in the composition is/are tissue, infectious disease, and/or patient-specific. For instance, the exact selection of peptides can be guided by expression patterns of the parent proteins in a given tissue or guided by mutation status of a patient. The selection can be dependent on the specific type of infectious disease, the type of organism causing the infectious disease (e.g., a pathogen, virus, bacteria, fungus, or a parasite), the status of the disease, earlier treatment regimens, the immune status of the patient, and, the HLA-haplotype of the patient. Furthermore, a vaccine can contain individualized components, according to personal needs of the particular patient. Examples include varying the selection of antigens according to the expression of the antigen in the particular patient or adjustments for secondary treatments following a first round or scheme of treatment.

A patient can be identified for administration of an antigen vaccine through the use of various diagnostic methods, e.g., patient selection methods described further below. Patient selection can involve identifying mutations in, or expression patterns of, one or more genes. In some cases, patient selection involves identifying the haplotype of the patient. The various patient selection methods can be performed in parallel, e.g., a sequencing diagnostic can identify both the mutations and the haplotype of a patient. The various patient selection methods can be performed sequentially, e.g., one diagnostic test identifies the mutations and separate diagnostic test identifies the haplotype of a patient, and where each test can be the same (e.g., both high-throughput sequencing) or different (e.g., one high-throughput sequencing and the other Sanger sequencing) diagnostic methods.

For a composition to be used as a vaccine for infectious diseases, antigens with similar normal self-peptides that are expressed in high amounts in normal tissues can be avoided or be present in low amounts in a composition described herein. On the other hand, if it is known that the infected cell of a patient expresses high amounts of a certain antigen, the respective pharmaceutical composition for treatment of this infection can be present in high amounts and/or more than one antigen specific for this particularly antigen or pathway of this antigen can be included.

Compositions comprising an antigen can be administered to an individual already suffering from an infectious disease. In therapeutic applications, compositions are administered to a patient in an amount sufficient to elicit an effective CTL response to the infectious disease organism antigen and to cure or at least partially arrest symptoms and/or complications. An amount adequate to accomplish this is defined as “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that compositions can generally be employed in serious infectious disease states, that is, life-threatening or potentially life threatening situations. In such cases, in view of the minimization of extraneous substances and the relative nontoxic nature of an antigen, it is possible and can be felt desirable by the treating physician to administer substantial excesses of these compositions.

For therapeutic use, administration can begin at the detection of an infection or prior to detection of an infection. This is followed by boosting doses until at least symptoms are substantially abated and for a period thereafter or immunity is considered to be provided (e.g., a memory B cell or T cell population, or antigen specific B cells or antibodies are produced).

The pharmaceutical compositions (e.g., vaccine compositions) for therapeutic treatment are intended for parenteral, topical, nasal, oral or local administration. A pharmaceutical compositions can be administered parenterally, e.g., intravenously, subcutaneously, intradermally, or intramuscularly. The compositions can be administered at the site of infection to induce a local immune response to the infection. Disclosed herein are compositions for parenteral administration which comprise a solution of the antigen and vaccine compositions are dissolved or suspended in an acceptable carrier, e.g., an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Antigens can also be administered via liposomes, which target them to a particular cells tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the antigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired antigen can be directed to the site of lymphoid cells, where the liposomes then deliver the selected therapeutic/immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

For therapeutic or immunization purposes, nucleic acids encoding a peptide and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation.

The nucleic acids can also be delivered complexed to cationic compounds, such as cationic lipids. Lipid-mediated gene delivery methods are described, for instance, in 9618372WOAWO 96/18372; 9324640WOAWO 93/24640; Mannino & Gould-Fogerite, BioTechniques 6(7): 682-691 (1988); U.S. Pat. No. 5,279,833 Rose U.S. Pat. Nos. 5,279,833; 9,106,309WOAWO 91/06309; and Felgner et al., Proc. Natl. Acad. Sci. USA 84: 7413-7414 (1987).

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the antigens, and thereby elicit a host immune (e.g., CTL) response against the infectious disease-derived peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

A means of administering nucleic acids uses minigene constructs encoding one or multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes can be improved by including synthetic (e.g. poly-alanine) or naturally-occurring flanking sequences adjacent to the CTL epitopes. The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Purified plasmid DNA can be prepared for injection using a variety of formulations. The simplest of these is reconstitution of lyophilized DNA in sterile phosphate-buffer saline (PBS). A variety of methods have been described, and new techniques can become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Also disclosed is a method of manufacturing an infectious disease vaccine, comprising performing the steps of a method disclosed herein; and producing an infectious disease vaccine comprising a plurality of antigens or a subset of the plurality of antigens.

Antigens disclosed herein can be manufactured using methods known in the art. For example, a method of producing an antigen or a vector (e.g., a vector including at least one sequence encoding one or more antigens) disclosed herein can include culturing a host cell under conditions suitable for expressing the antigen or vector wherein the host cell comprises at least one polynucleotide encoding the antigen or vector, and purifying the antigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NS0 cell, yeast, or a HEK293 cell. Host cells can be transformed with one or more polynucleotides comprising at least one nucleic acid sequence that encodes an antigen or vector disclosed herein, optionally wherein the isolated polynucleotide further comprises a promoter sequence operably linked to the at least one nucleic acid sequence that encodes the antigen or vector. In certain embodiments the isolated polynucleotide can be cDNA.

### VII. Presentation Identification System

### VII.A. System Overview

FIG. 1 is an overview of an environment for identifying likelihoods of peptide presentation in patients, in accordance with an embodiment. The environment 100 provides context in order to introduce a presentation identification system 160, itself including a presentation information store 165.

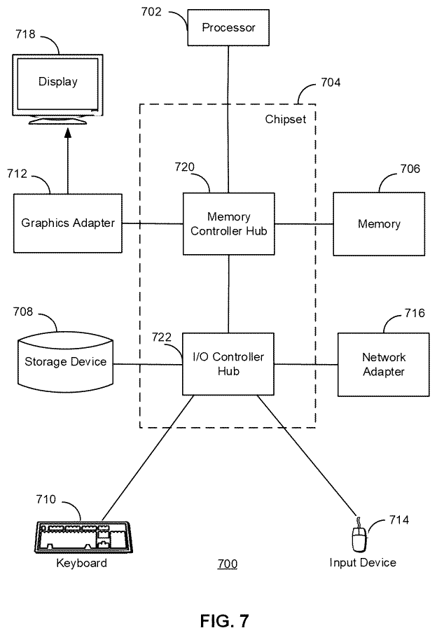

The presentation identification system 160 is one or more computer models, embodied in a computing system as discussed below with respect to FIG. 7, that receives peptide sequences (e.g., infectious disease-derived peptide sequences) associated with a set of MHC alleles and determines likelihoods that the infectious disease-derived peptide sequences will be presented by one or more of the set of associated MHC alleles. This is useful in a variety of contexts. One specific use case for the presentation identification system 160 is that it is able to receive nucleotide sequences of candidate antigens (e.g., candidate antigen sequences 114) associated with a set of MHC alleles expressed by the patient 110 and determine likelihoods that the candidate antigens will be presented by one or more of the associated MHC alleles of the patient 110 and/or induce immunogenic responses in the immune system of the patient 110. Those candidate antigens with high likelihoods as determined by system 160 can be selected for development of a therapeutic 118 (e.g., inclusion in a vaccine, development of TCRs specific for the selected antigens, and/or development of antibodies exhibiting binding affinity for the selected antigens). Thus, the therapeutic, if administered, can elicit an anti-infectious disease immune response from the immune system of the patient 110.

The presentation identification system 160 determines presentation likelihoods through one or more presentation models, herein also referred to as a multi-part presentation model. Specifically, the presentation models generate likelihoods of whether given peptide sequences will be presented for a set of associated MHC alleles, and are generated based on presentation information stored in store 165. For example, the presentation models may generate likelihoods of whether a peptide sequence “YVYVADVAAK” will be presented for the set of alleles HLA-A*02:01, HLA-B*07:02, HLA-B*08:03, HLA-C*01:04, HLA-A*06:03, HLA-B*01:04 on the cell surface of the sample. The presentation information 165 contains information on whether peptides bind to different types of MHC alleles such that those peptides are presented by MHC alleles, which in the models is determined depending on positions of amino acids in the peptide sequences. The presentation model can predict whether an unrecognized peptide sequence will be presented in association with an associated set of MHC alleles based on the presentation information 165.

### VII.B. Presentation Information

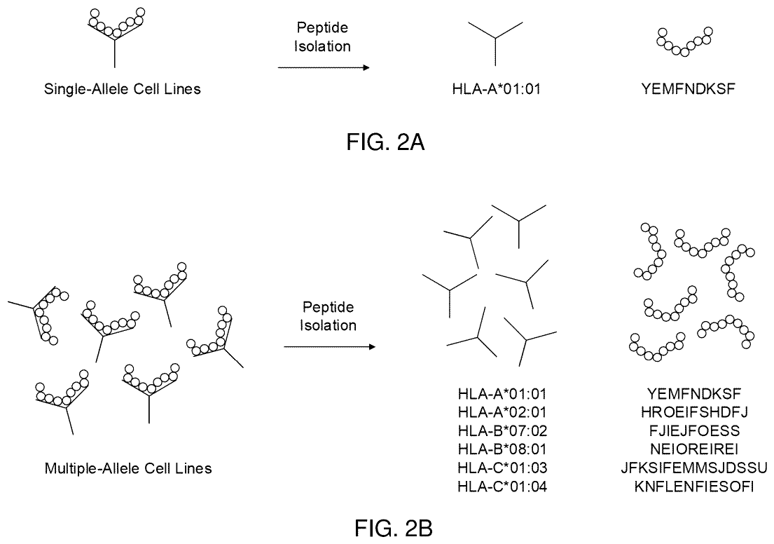

FIGS. 2A and 2B illustrate a method of obtaining presentation information, in accordance with an embodiment. In various embodiments, the presentation information 165 includes two general categories of information: allele-interacting information and allele-noninteracting information. Allele-interacting information includes information that influence presentation of peptide sequences that are dependent on the type of MHC allele. Allele-noninteracting information includes information that influence presentation of peptide sequences that are independent on the type of MHC allele.

### VII.B.1. Allele-Interacting Information

Allele-interacting information may include identified peptide sequences that are known to have been presented by one or more identified MHC molecules from humans, mice, etc. Notably, this may or may not include data obtained from infectious disease samples. The presented peptide sequences may be identified from cells that express a single MHC allele. In various embodiments, the presented peptide sequences are collected from single-allele cell lines that are engineered to express a predetermined MHC allele and that are subsequently exposed to a synthetic protein. Peptides presented on the MHC allele are isolated by techniques such as acid-elution and identified through mass spectrometry. FIG. 2A shows an example of this, where the example peptide YEMFNDKS, presented on the predetermined MHC allele HLA-A*01:01, is isolated and identified through mass spectrometry. Since peptides are identified through cells engineered to express a single predetermined MHC protein, the direct association between a presented peptide and the MHC protein to which it was bound to is definitively known.

The presented peptide sequences may also be collected from cells that express multiple MHC alleles. In humans, 6 different types of MHC molecules are expressed for a cell. Such presented peptide sequences may be identified from multiple-allele cell lines that are engineered to express multiple predetermined MHC alleles. Such presented peptide sequences may also be identified from tissue samples, either from normal tissue samples or tissue samples exposed to one of a pathogen, virus, bacteria, fungus, or a parasite capable of causing an infectious disease. In this case, the MHC molecules can be immunoprecipitated from normal or infectious disease tissue. Peptides presented on the multiple MHC alleles can similarly be isolated by techniques such as acid-elution and identified through mass spectrometry. FIG. 2B shows an example of this, where the six example peptides, YEMFNDKSF, HROEIFSHDFJ, FJIEJFOESS, NEIOREIREI, JFKSIFEMMSJDSSU, and KNFLENFIESOFI, are presented on identified MHC alleles HLA-A*01:01, HLA-A*02:01, HLA-B*07:02, HLA-B*08:01, HLA-C*01:03, and HLA-C*01:04 and are isolated and identified through mass spectrometry. In contrast to single-allele cell lines, the direct association between a presented peptide and the MHC protein to which it was bound to may be unknown since the bound peptides are isolated from the MHC molecules before being identified.

Allele-interacting information can also include mass spectrometry ion current which depends on both the concentration of peptide-MHC molecule complexes, and the ionization efficiency of peptides. The ionization efficiency varies from peptide to peptide in a sequence-dependent manner. Generally, ionization efficiency varies from peptide to peptide over approximately two orders of magnitude, while the concentration of peptide-MHC complexes varies over a larger range than that.

Allele-interacting information can also include measurements or predictions of binding affinity between a given MHC allele and a given peptide. One or more affinity models can generate such predictions. For example, presentation information 165 may include a binding affinity prediction of 1000 nM between the peptide YEMFNDKSF and the allele HLA-A*01:01. Few peptides with IC50>1000 nm are presented by the MHC, and lower IC50 values increase the probability of presentation.

Allele-interacting information can also include measurements or predictions of stability of the MHC complex. One or more stability models that can generate such predictions. For example, going back to the example shown in FIG. 2B, presentation information 165 may include a stability prediction of a half-life of 1 h for the molecule HLA-A*01:01.

Allele-interacting information can also include the measured or predicted rate of the formation reaction for the peptide-MHC complex. Complexes that form at a higher rate are more likely to be presented on the cell surface at high concentration.

Allele-interacting information can also include the sequence and length of the peptide. MHC class I molecules typically prefer to present peptides with lengths between 8 and 15 peptides. 60-80% of presented peptides have length 9.

Allele-interacting information can also include the presence of kinase sequence motifs on the antigen encoded peptide, and the absence or presence of specific post-translational modifications on the antigen encoded peptide. The presence of kinase motifs affects the probability of post-translational modification, which may enhance or interfere with MHC binding.

Allele-interacting information can also include the expression or activity levels of proteins involved in the process of post-translational modification, e.g., kinases (as measured or predicted from RNA seq, mass spectrometry, or other methods).

Allele-interacting information can also include the probability of presentation of peptides with similar sequence in cells from other individuals expressing the particular MHC allele as assessed by mass-spectrometry proteomics or other means.

Allele-interacting information can also include the expression levels of the particular MHC allele in the individual in question (e.g. as measured by RNA-seq or mass spectrometry). Peptides that bind most strongly to an MHC allele that is expressed at high levels are more likely to be presented than peptides that bind most strongly to an MHC allele that is expressed at a low level.

Allele-interacting information can also include the overall antigen encoded peptide-sequence-independent probability of presentation by the particular MHC allele in other individuals who express the particular MHC allele.

Allele-interacting information can also include the overall peptide-sequence-independent probability of presentation by MHC alleles in the same family of molecules (e.g., HLA-A, HLA-B, HLA-C, HLA-DQ, HLA-DR, HLA-DP) in other individuals. For example, HLA-C molecules are typically expressed at lower levels than HLA-A or HLA-B molecules, and consequently, presentation of a peptide by HLA-C is a priori less probable than presentation by HLA-A or HLA-B 11.

Allele-interacting information can also include the protein sequence of the particular MHC allele.

Any MHC allele-noninteracting information listed in the below section can also be modeled as an MHC allele-interacting information.

### VII.B.2. Allele-Noninteracting Information

Allele-noninteracting information can include C-terminal sequences flanking the antigen encoded peptide within its source protein sequence. C-terminal flanking sequences may impact proteasomal processing of peptides. However, the C-terminal flanking sequence is cleaved from the peptide by the proteasome before the peptide is transported to the endoplasmic reticulum and encounters MHC alleles on the surfaces of cells. Consequently, MHC molecules receive no information about the C-terminal flanking sequence, and thus, the effect of the C-terminal flanking sequence cannot vary depending on MHC allele type. For example, going back to the example shown in FIG. 2B, presentation information 165 may include the C-terminal flanking sequence FOEIFNDKSLDKFJI of the presented peptide FJIEJFOESS identified from the source protein of the peptide.

Allele-noninteracting information can also include mRNA quantification measurements. For example, mRNA quantification data can be obtained for the same samples that provide the mass spectrometry training data. In one embodiment, the mRNA quantification measurements are identified from software tool RSEM. Detailed implementation of the RSEM software tool can be found at Bo Li and Colin N. Dewey. RSEM: accurate transcript quantification from RNA-Seq data with or without a reference genome. BMC Bioinformatics, 12:323, August 2011. In one embodiment, the mRNA quantification is measured in units of fragments per kilobase of transcript per Million mapped reads (FPKM).

Allele-noninteracting information can also include the N-terminal sequences flanking the peptide within its source protein sequence.

In particular embodiments, allele-noninteraction information includes both the C-terminal sequences flanking the peptide within its source protein sequence and the N-terminal sequences flanking the peptide within its source protein sequence.

Allele-noninteracting information can also include the presence of protease cleavage motifs in the peptide. Peptides that contain protease cleavage motifs are less likely to be presented, because they will be more readily degraded by proteases, and will therefore be less stable within the cell.

Allele-noninteracting information can also include the turnover rate of the source protein as measured in the appropriate cell type. Faster turnover rate (i.e., lower half-life) increases the probability of presentation; however, the predictive power of this feature is low if measured in a dissimilar cell type.

Allele-noninteracting information can also include the length of the source protein.

Allele-noninteracting information can also include the level of expression of the proteasome, immunoproteasome, thymoproteasome, or other proteases. Different proteasomes have different cleavage site preferences. More weight will be given to the cleavage preferences of each type of proteasome in proportion to its expression level.

Allele-noninteracting information can also include the expression of the source gene of the peptide (e.g., as measured by RNA-seq or mass spectrometry). Peptides from more highly expressed genes are more likely to be presented. Peptides from genes with undetectable levels of expression can be excluded from consideration.

Allele-noninteracting information can also include the probability that the source mRNA of the antigen encoded peptide will be subject to nonsense-mediated decay as predicted by a model of nonsense-mediated decay, for example, the model from Rivas et al, Science 2015.

Allele-noninteracting information can also include the typical tissue-specific expression of the source gene of the peptide during various stages of the cell cycle. Genes that are expressed at a low level overall (as measured by RNA-seq or mass spectrometry proteomics) but that are known to be expressed at a high level during specific stages of the cell cycle are likely to produce more presented peptides than genes that are stably expressed at very low levels.

Allele-noninteracting information can also include a comprehensive catalog of features of the source protein as given in e.g. uniProt or PDB http://www.rcsb.org/pdb/home/home.do. These features may include, among others: the secondary and tertiary structures of the protein, subcellular localization 11, Gene ontology (GO) terms. Specifically, this information may contain annotations that act at the level of the protein, e.g., 5′ UTR length, and annotations that act at the level of specific residues, e.g., helix motif between residues 300 and 310. These features can also include turn motifs, sheet motifs, and disordered residues.

Allele-noninteracting information can also include features describing the properties of the domain of the source protein containing the peptide, for example: secondary or tertiary structure (e.g., alpha helix vs beta sheet); Alternative splicing.

Allele-noninteracting information can also include features describing the presence or absence of a presentation hotspot at the position of the peptide in the source protein of the peptide.

Allele-noninteracting information can also include the probability of presentation of peptides from the source protein of the peptide in question in other individuals (after adjusting for the expression level of the source protein in those individuals and the influence of the different HLA types of those individuals).

Allele-noninteracting information can also include the probability that the peptide will not be detected or over-represented by mass spectrometry due to technical biases.

Allele-noninteracting information can also include the probability that the peptide binds to the TAP or the measured or predicted binding affinity of the peptide to the TAP. Peptides that are more likely to bind to the TAP, or peptides that bind the TAP with higher affinity are more likely to be presented.

Allele-noninteracting information can also include known functionality of HLA alleles, as reflected by, for instance HLA allele suffixes. For example, the N suffix in the allele name HLA-A*24:09N indicates a null allele that is not expressed and is therefore unlikely to present epitopes; the full HLA allele suffix nomenclature is described at https://www.ebi.ac.uk/ipd/imgt/hla/nomenclature/suffixes.html.

### VII.C. Presentation Identification System

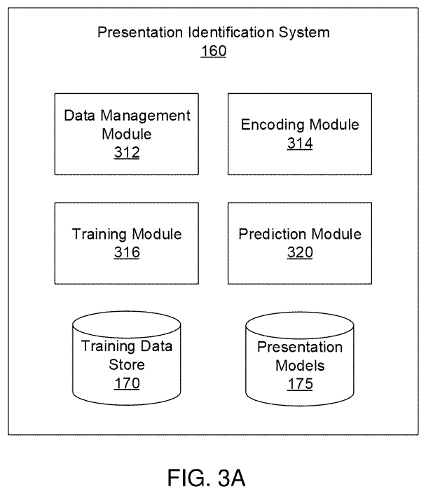

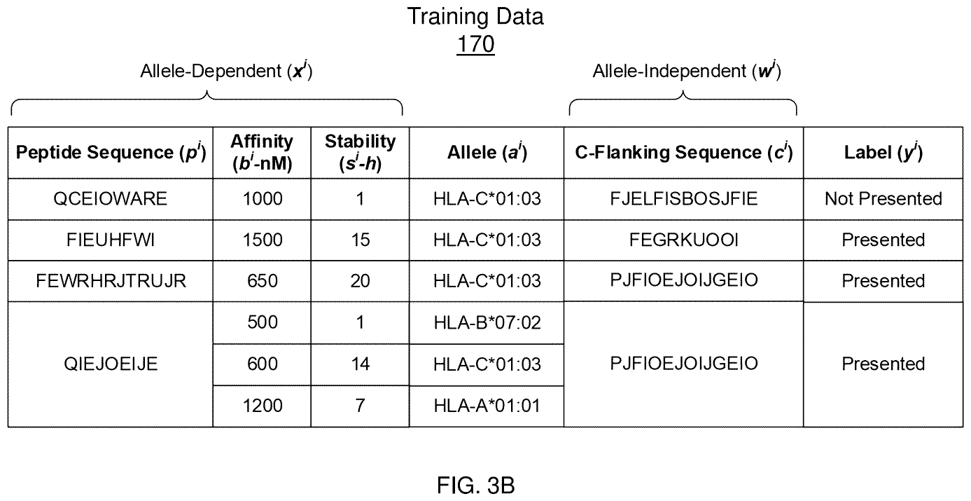

FIG. 3A is a high-level block diagram illustrating the computer logic components of the presentation identification system 160, according to one embodiment. In this example embodiment, the presentation identification system 160 includes a data management module 312, an encoding module 314, a training module 316, and a prediction module 320. The presentation identification system 160 is also comprised of a training data store 170A and a presentation models store 175. Some embodiments of the model management system 160 have different modules than those described here. Similarly, the functions can be distributed among the modules in a different manner than is described here.

### VII.C.1. Data Management Module

The data management module 312 generates sets of training data from the presentation information 165. Each set of training data contains a plurality of data instances, in which each data instance i contains a set of independent variables zⁱ that include at least a presented or non-presented peptide sequence pⁱ, one or more associated MHC alleles aⁱ associated with the peptide sequence pⁱ, and a dependent variable yⁱ that represents information that the presentation identification system 160 is interested in predicting for new values of independent variables.

In one particular implementation referred throughout the remainder of the specification, the dependent variable yⁱ is a binary label indicating whether peptide pⁱ was presented by the one or more associated MHC alleles aⁱ. However, it is appreciated that in other implementations, the dependent variable yⁱ can represent any other kind of information that the presentation identification system 160 is interested in predicting dependent on the independent variables zⁱ. For example, in another implementation, the dependent variable yⁱ may also be a numerical value indicating the mass spectrometry ion current identified for the data instance.

The peptide sequence pⁱ for data instance i is a sequence of kᵢ amino acids, in which kᵢ may vary between data instances i within a range. For example, that range may be 8-15 for MHC class I or 9-30 for MHC class II. In one specific implementation of system 160, all peptide sequences pⁱ in a training data set may have the same length, e.g. 9. The number of amino acids in a peptide sequence may vary depending on the type of MHC alleles (e.g., MHC alleles in humans, etc.). The MHC alleles aⁱ for data instance i indicate which MHC alleles were present in association with the corresponding peptide sequence pⁱ.

The data management module 312 may also include additional allele-interacting variables, such as binding affinity bⁱ and stability sⁱ predictions in conjunction with the peptide sequences pⁱ and associated MHC alleles aⁱ contained in the training data 170. For example, the training data 170 may contain binding affinity predictions bⁱ between a peptide pⁱ and each of the associated MHC molecules indicated in aⁱ. As another example, the training data 170 may contain stability predictions sⁱ for each of the MHC alleles indicated in aⁱ.

In particular embodiments, the data management module 312 includes 1) a label indicating whether peptide pⁱ was presented by the one or more associated MHC alleles aⁱ as determined via mass spectrometry, and 2) additional binding affinity bⁱ predictions in conjunction with the peptide sequences pⁱ and associated MHC alleles aⁱ. This enables training of presentation models (e.g., multi-part presentation models) using training data derived from both 1) binding affinity data between peptide sequences and HLA alleles and 2) eluted peptide data from mass spectrometry representing presentation of peptide sequences and HLA alleles. Here, it may be preferable to use both types of data to train the multi-part presentation model, especially in scenarios where eluted peptide data of peptide sequences generated via mass spectrometry are limited in quantity.

In various embodiments, the peptide sequences (e.g., training peptide sequences) used to train the multi-part presentation model are infectious disease-derived peptides. Thus, the multi-part presentation model can be trained to accurately predict whether infectious disease-derived peptides are likely to be presented or not presented by one or more HLA alleles. In various embodiments, the peptide sequences (e.g., training peptide sequences) used to train the multi-part presentation model are human peptides. In various embodiments, training peptide sequences used to train the multi-part presentation model include both infectious disease-derived peptides as well as human peptides. In such embodiments, the human peptides may represent additional training peptide sequences for training the multi-part presentation model if infectious disease-derived peptide sequences are limited in quantity.

In particular embodiments, the training data for training the multi-part presentation model includes binding affinity data between infectious disease-derived peptide sequences and HLA alleles. In particular embodiments, the training data for training the multi-part presentation model includes eluted human peptide data (e.g., human immunopeptidomics) from mass spectrometry representing presentation of human peptide sequences and HLA alleles. In particular embodiments, the training data for training the multi-part presentation model includes 1) binding affinity data between infectious disease-derived peptide sequences and HLA alleles and 2) eluted human peptide data (e.g., human immunopeptidomics) from mass spectrometry representing presentation of human peptide sequences and HLA alleles. This situation is beneficial where there is limited eluted peptide data for infectious disease-derived peptide sequences. Thus, the eluted human peptide data are used to supplement the training of the multi-part presentation model.

In various embodiments, the data management module 312 may also include allele-noninteracting variables wⁱ, such as C-terminal flanking sequences and mRNA quantification measurements in conjunction with the peptide sequences pⁱ.

The data management module 312 may also identifies peptide sequences that are not presented by MHC alleles to generate the training data 170. For example, this involves identifying the “longer” sequences of source protein that include presented peptide sequences prior to presentation. When the presentation information contains engineered cell lines, the data management module 312 identifies a series of peptide sequences in the synthetic protein to which the cells were exposed to that were not presented on MHC alleles of the cells. When the presentation information contains tissue samples, the data management module 312 identifies source proteins from which presented peptide sequences originated from, and identifies a series of peptide sequences in the source protein that were not presented on MHC alleles of the tissue sample cells.

In various embodiments, the data management module 312 may also artificially generate peptides with random sequences of amino acids and identify the generated sequences as peptides not presented on MHC alleles. This can be accomplished by randomly generating peptide sequences allows the data management module 312 to easily generate large amounts of synthetic data for peptides not presented on MHC alleles. Since in reality, a small percentage of peptide sequences are presented by MHC alleles, the synthetically generated peptide sequences are highly likely not to have been presented by MHC alleles even if they were included in proteins processed by cells.

In various embodiments, the data management module 312 artificially generates peptides to balance the training dataset so as to minimize bias arising from an unbalanced training dataset. For example, a training dataset may have a number of peptides that were presented by MHC alleles (M₁) and a number of peptides that were not presented by MHC alleles (M₂). In various embodiments, if the training dataset has fewer peptides that were not presented by MHC alleles in comparison to the number of peptides that were presented by MHC alleles (e.g., M₂<M₁), the data management module 312 artificially generates peptides that were not presented by MHC alleles to balance the training dataset. In various embodiments, the data management module 312 artificially generates peptides that were not presented by MHC alleles if the ratio of peptides not presented by MHC alleles to peptides presented by MHC alleles (M₂/M₁) is less than a threshold value (N). The threshold value N can be any of 1, 5, 10, 20, 30, 40, 50, 60, 70, 80, 90, or 100. In particular embodiments, the threshold value N is 50. Therefore, if the ratio of peptides not presented by MHC alleles to peptides presented by MHC alleles (M₂/M₁) is less than 50, the data management module 312 artificially generates peptides that were not presented by MHC alleles to further balance the training dataset. In such embodiments, balancing the dataset ensures that there are significantly more peptides not presented by MHC alleles in the training dataset such that the presentation model is adequately trained to recognize peptides that are not presented by MHC alleles.

In various embodiments, the training dataset includes binding affinity data between infectious disease-derived peptide sequences and HLA alleles. Thus, in such embodiments, the data management module 312 artificially generates peptides that were not presented by MHC alleles if the ratio of peptides not presented by MHC alleles (as indicated by binding affinity value) to peptides presented (as indicated by binding affinity value) by MHC alleles (M₂/M₁) is less than a threshold value (N). In various embodiments, the training dataset includes eluted human peptide data (e.g., human immunopeptidomics) from mass spectrometry representing presentation of human peptide sequences and HLA alleles. Thus, in such embodiments, the data management module 312 artificially generates peptides that were not presented by MHC alleles if the ratio of peptides not presented by MHC alleles to peptides presented by MHC alleles (M₂/M₁) is less than a threshold value (N).

In various embodiments, the training dataset includes both 1) binding affinity data between infectious disease-derived peptide sequences and HLA alleles and 2) eluted human peptide data (e.g., human immunopeptidomics) from mass spectrometry representing presentation of human peptide sequences and HLA alleles. Thus, in such embodiments, the data management module 312 may artificially generate peptides in both the binding affinity data and/or the eluted human peptide data. For example, if the ratio of peptides not presented by MHC alleles (as indicated by binding affinity value) to peptides presented (as indicated by binding affinity value) by MHC alleles (M₂/M₁) is less than a threshold value (N), then the data management module 312 artificially generates peptides that were not presented by MHC alleles (as indicated by binding affinity value) to further balance the training dataset.

As another example, if the ratio of peptides not presented by MHC alleles (as indicated in the eluted human peptide data) to peptides presented (as indicated in the eluted human peptide data) by MHC alleles (M₂/M₁) is less than a threshold value (N), then the data management module 312 artificially generates peptides that were not presented by MHC alleles (in the eluted human peptide data) to further balance the training dataset. The threshold value N can be any of 1, 5, 10, 20, 30, 40, 50, 60, 70, 80, 90, or 100. In particular embodiments, the threshold value N is 50.

In various embodiments, the data management module 312 artificially generates peptides of varying lengths. In various embodiments, the data management module 312 artificially generates peptides by drawing from a length distribution matching a source dataset's peptide length distribution. In various embodiments, the data management module 312 artificially generates peptides by sampling a same or similar HLA genotype frequency as the source dataset's HLA genotype distribution. A source dataset may refer to a dataset from which the training dataset was originally obtained from. After artificially generating peptide sequences, the generated peptides are combined with the original training dataset. For example, the data management module 312 appends the generated peptides to the original training dataset prior to shuffling the training dataset to ensure that the training data are sampled randomly when used for training the presentation model.

FIG. 3B illustrates an example set of training data 170, according to one embodiment. Specifically, the first 3 data instances in the training data 170 indicate peptide presentation information from a single-allele cell line involving the allele HLA-C*01:03 and 3 peptide sequences QCEIOWARE, FIEUHFWI, and FEWRHRJTRUJR. The fourth data instance in the training data 170 indicates peptide information from a multiple-allele cell line involving the alleles HLA-B*07:02, HLA-C*01:03, HLA-A*01:01 and a peptide sequence QIEJOEIJE. The first data instance indicates that peptide sequence QCEIOWARE was not presented by the allele HLA-C*01:03. As discussed in the prior two paragraphs, the peptide sequence may be randomly generated by the data management module 312 or identified from source protein of presented peptides. The training data 170 also includes a binding affinity prediction of 1000 nM and a stability prediction of a half-life of 1 h for the peptide sequence-allele pair. The training data 170 also includes allele-noninteracting variables, such as the C-terminal flanking sequence of the peptide FJELFISBOSJFIE, and a mRNA quantification measurement of 102 FPKM. The fourth data instance indicates that peptide sequence QIEJOEIJE was presented by one of the alleles HLA-B*07:02, HLA-C*01:03, or HLA-A*01:01. The training data 170 also includes binding affinity predictions and stability predictions for each of the alleles, as well as the C-flanking sequence of the peptide and the mRNA quantification measurement for the peptide.

### VII.C.2. Encoding Module

In various embodiments, the encoding module 314 encodes information contained in the training data 170 into a representation (e.g., a numerical representation) that can be used to generate the one or more presentation models. As one example, the encoding module 314 encodes peptide sequences of a candidate infectious disease-derived peptide into a representation for analysis by one or more presentation models. As another example, the encoding module 314 encodes peptide sequences of HLA alleles into a representation for use in generating presentation likelihoods. In various embodiments, the encoding module 314 encodes information contained in the training data 170 into one or more numerical vectors that are inputted into the one or more presentation models.

In one implementation, the encoding module 314 one-hot encodes sequences (e.g., peptide sequences, C-terminal flanking sequences, N-terminal flanking sequences, or peptide sequences of MHC alleles) over a predetermined 20-letter amino acid alphabet. Specifically, a peptide sequence pⁱ with kᵢ amino acids is represented as a row vector of 20−kᵢ elements, where a single element among pⁱ₂₀₍ⱼ₋₁₎₊₁, pⁱ₂₀₋₍ⱼ₋₁₎₊₂, . . . , pⁱ₂₀₋ⱼ that corresponds to the alphabet of the amino acid at the j-th position of the peptide sequence has a value of 1. Otherwise, the remaining elements have a value of 0. As an example, for a given alphabet {A, C, D, E, F, G, H, I, K, L, M, N, P, Q, R, S, T, V, W, Y}, the peptide sequence EAF of 3 amino acids for data instance i may be represented by the row vector of 60 elements pⁱ=[0 0 0 1 0 0 0 0 0 0 0 0 0 0 0 0 0 0 0 0 1 0 0 0 0 0 0 0 0 0 0 0 0 0 0 0 0 0 0 0 0 0 0 0 1 0 0 0 0 0 0 0 0 0 0 0 0 0 0 0]. The C-terminal flanking sequence cⁱ can be similarly encoded as described above, as well as the protein sequence dₕ for MHC alleles, and other sequence data in the presentation information.

When the training data 170 contains sequences of differing lengths of amino acids, the encoding module 314 may further encode the peptides into equal-length vectors by adding a PAD character to extend the predetermined alphabet. For example, this may be performed by left-padding the peptide sequences with the PAD character until the length of the peptide sequence reaches the peptide sequence with the greatest length in the training data 170. Thus, when the peptide sequence with the greatest length has kₘₐₓ amino acids, the encoding module 314 numerically represents each sequence as a row vector of (20+1)·kₘₐₓ elements. As an example, for the extended alphabet {PAD, A, C, D, E, F, G, H, I, K, L, M, N, P, Q, R, S, T, V, W, Y} and a maximum amino acid length of kₘₐₓ=5, the same example peptide sequence EAF of 3 amino acids may be represented by the row vector of 105 elements pⁱ=[1 0 0 0 0 0 0 0 0 0 0 0 0 0 0 0 0 0 0 0 0 1 0 0 0 0 0 0 0 0 0 0 0 0 0 0 0 0 0 0 0 0 0 0 0 0 1 0 0 0 0 0 0 0 0 0 0 0 0 0 0 0 0 0 1 0 0 0 0 0 0 0 0 0 0 0 0 0 0 0 0 0 0 0 0 0 0 0 0 1 0 0 0 0 0 0 0 0 0 0 0 0 0 0 0]. The C-terminal flanking sequence c or other sequence data can be similarly encoded as described above. Thus, each independent variable or column in the peptide sequence pⁱ or cⁱ represents presence of a particular amino acid at a particular position of the sequence.

Although the above method of encoding sequence data was described in reference to sequences having amino acid sequences, the method can similarly be extended to other types of sequence data, such as DNA or RNA sequence data, and the like.

The encoding module 314 also encodes the one or more MHC alleles aᵢ for data instance i as a row vector of m elements, in which each element h=1, 2, . . . , m corresponds to a unique identified MHC allele. The elements corresponding to the MHC alleles identified for the data instance i have a value of 1. Otherwise, the remaining elements have a value of 0. As an example, the alleles HLA-B*07:02 and HLA-C*01:03 for a data instance i corresponding to a multiple-allele cell line among m=4 unique identified MHC allele types {HLA-A*01:01, HLA-C*01:08, HLA-B*07:02, HLA-C*01:03} may be represented by the row vector of 4 elements aⁱ=[0 0 1 1], in which a₃ⁱ=1 and a₄ⁱ=1. Although the example is described herein with 4 identified MHC allele types, the number of MHC allele types can be hundreds or thousands in practice. As previously discussed, each data instance i typically contains at most 6 different MHC allele types in association with the peptide sequence pᵢ.

The encoding module 314 also encodes the label yᵢ for each data instance i as a binary variable having values from the set of {0, 1}, in which a value of 1 indicates that peptide xⁱ was presented by one of the associated MHC alleles aⁱ and a value of 0 indicates that peptide xⁱ was not presented by any of the associated MHC alleles aⁱ. When the dependent variable yᵢ represents the mass spectrometry ion current, the encoding module 314 may additionally scale the values using various functions, such as the log function having a range of [−∞, ∞] for ion current values between [0, ∞].

The encoding module 314 may represent a pair of allele-interacting variables xₕⁱ for peptide pᵢ and an associated MHC allele h as a row vector in which numerical representations of allele-interacting variables are concatenated one after the other. For example, the encoding module 314 may represent xₕⁱ as a row vector equal to [pⁱ], [pⁱ bₕⁱ], [pⁱ sₕⁱ], or [pⁱ bₕⁱ sₕⁱ], where bₕⁱ is the binding affinity prediction for peptide pᵢ and associated MHC allele h, and similarly for sₕⁱ for stability. Alternatively, one or more combination of allele-interacting variables may be stored individually (e.g., as individual vectors or matrices).

In one instance, the encoding module 314 represents binding affinity information by incorporating measured or predicted values for binding affinity in the allele-interacting variables xₕⁱ.

In one instance, the encoding module 314 represents binding stability information by incorporating measured or predicted values for binding stability in the allele-interacting variables xₕⁱ,

In one instance, the encoding module 314 represents binding on-rate information by incorporating measured or predicted values for binding on-rate in the allele-interacting variables xₕⁱ.

Additional examples of training data that the encoding module 314 may encode are described in WO2017106638 and WO2019168984, each of which is incorporated by reference in its entirety.

### VIII. Training Module

The training module 316 constructs one or more presentation models, such as one or more multi-part presentation models, that generate likelihoods of whether peptide sequences (e.g., infectious disease-derived peptide sequences) will be presented by MHC alleles associated with the peptide sequences. Specifically, given a peptide sequence p^{k} and a set of MHC alleles a^{k} and/or MHC allele sequences d^{k} associated with the peptide sequence p^{k}, a multi-part presentation model generates an estimate uₖ indicating a likelihood that the peptide sequence p^{k} will be presented by one or more of the associated MHC alleles a^{k}.

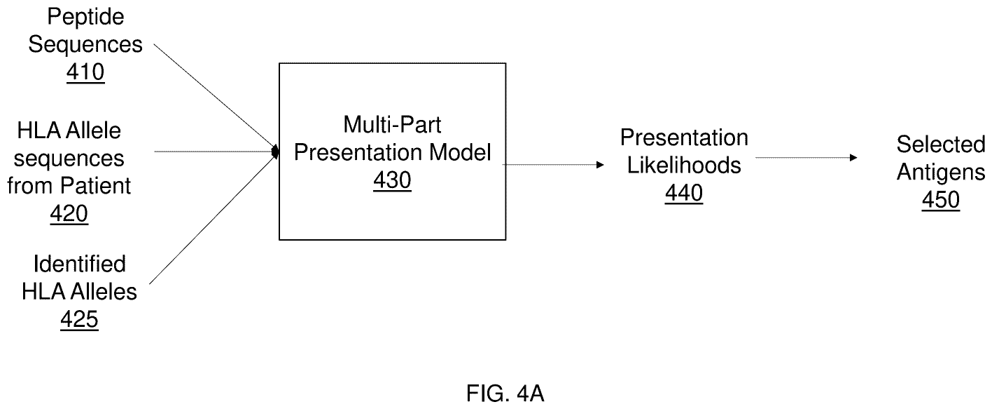

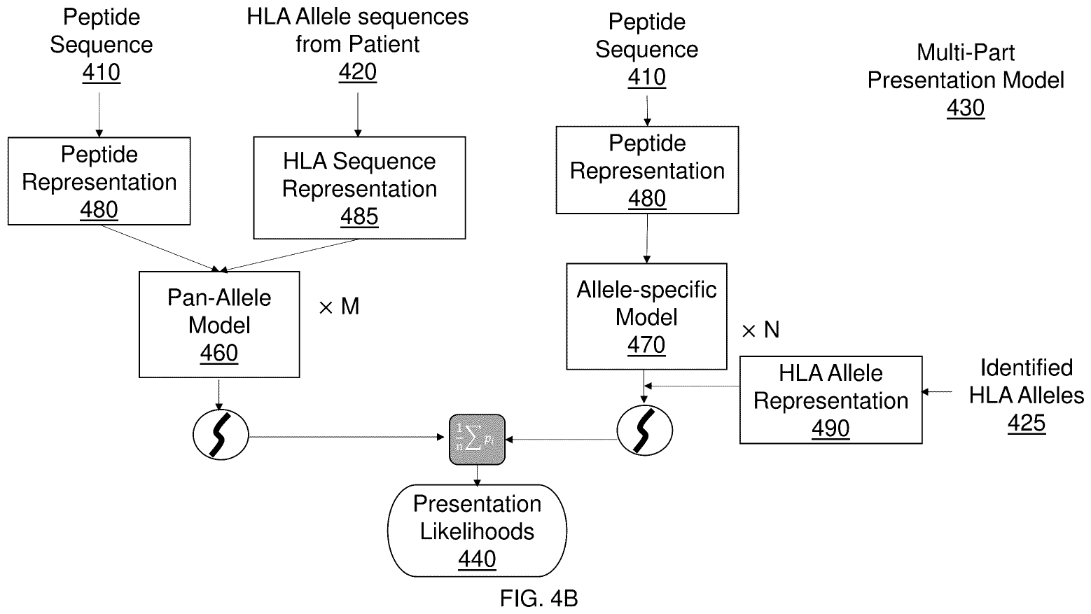

Reference is now made to FIG. 4A, which represents a flow process for implementing the multi-part presentation model, according to one embodiment. As shown in FIG. 4A, the multi-part presentation model 430 receives, as input, one or more peptide sequences 410 (e.g., infectious disease-derived peptide sequences), HLA allele sequences from the patient 420 (e.g., sequences of HLA alleles expressed by the patient), and/or identified HLA alleles 425 of the patient. Although FIG. 4A shows both HLA allele sequences from the patient 420 and identified HLA alleles 425 of the patient, in various embodiments, only the HLA allele sequences from the patient 420 are needed. For example, the identified HLA alleles 425 may be derived from the HLA allele sequences, given an HLA allele sequence that is sufficiently distinct and attributable to only one HLA allele.

Generally, the multi-part presentation model 430 was previously trained using training data. In particular embodiments, the training data for training the multi-part presentation model includes 1) binding affinity data between infectious disease-derived peptide sequences and HLA alleles and 2) eluted human peptide data (e.g., human immunopeptidomics) from mass spectrometry representing presentation of human peptide sequences and HLA alleles. This situation is beneficial where there is limited eluted peptide data for infectious disease-derived peptide sequences.

Generally, the multi-part presentation model 430 analyzes at least the peptide sequences 410 and the HLA allele sequences from the patient 420 and determines a set of presentation likelihoods 440. Here, each presentation likelihood may represent the likelihood that a particular peptide sequence will be presented by the HLA alleles expressed by the patient. Based on the presentation likelihoods 440, antigens are identified to generate selected antigens 450. For example, a threshold number of peptide sequences with the highest presentation likelihood 440 values are included as selected antigens, which can be used to develop a therapeutic, as described in further detail herein.

### VIII.A. Overview of Multi-Part Presentation Model

The training module 316 constructs the one more multi-part presentation models based on the training data sets stored in store 170 generated from the presentation information stored in 165.

Reference is now made to FIG. 4B, which shows the network architecture of the multi-part presentation model in further detail, according to one embodiment. The multi-part presentation model includes a first part comprising at least a pan-allele model 460 portion (also referred to as a pan-specific model portion) and a second part comprising a plurality of allele-specific models 470. Generally, the pan-allele model 460 and the allele-specific model 470 analyze different information as input, but both output per-allele presentation likelihoods. At a high level, the pan-allele model analyzes both peptide sequences and HLA sequences to generate per-allele presentation likelihoods. Thus, the pan-allele model architecture represents alleles as HLA sequences instead of categorical values, thereby allowing similar alleles to share information, but blurs the distinction between alleles. In contrast, allele-specific models analyze peptide sequences without analyzing HLA sequences. In various embodiments, allele-specific models may analyze additional information, such as allele-noninteracting information, an example of which include C-flanking or N-flanking sequences. Here, allele-specific models treat each allele independent of all other alleles and therefore, each instance of an allele-specific model learns from all alleles in the training dataset, but similar HLA alleles are learned independently from each other. By leveraging a multi-part presentation model 430 that includes both pan-allele models 460 and allele-specific models 470, the multi-part presentation model 430 is able to predict per-allele presentation likelihoods with improved accuracy.

Referring first to the pan-allele model 460 (also referred to as a pan-specific model), in various embodiments, the pan-allele model 460 may receive, as input, a representation of the peptide sequence 410, which is shown in FIG. 4B as a peptide representation 480. In various embodiments, the peptide sequence 410 is from an infectious disease-derived peptide. In various embodiments, the peptide sequence 410 is from a human peptide. As described herein, the peptide representation 480 may be a representation of the peptide sequence 410 generated by the encoding module 314. Thus, the pan-allele model 460 is trained to appropriately analyze the peptide representation 480. In some embodiments, the pan-allele model 460 directly receives, as input, the peptide sequence 410. Therefore, the peptide sequence 410 need not undergo a transformation into a peptide representation prior to being input into the pan-allele model 460.

In various embodiments, the pan-allele model 460 may receive, as input, a representation of the HLA sequences from the patient 420, which is shown in FIG. 4B as a HLA sequence representation 485. As described herein, the HLA sequence representation 485 may be a representation of the HLA sequences from the patient 420 generated by the encoding module 314. Thus, the pan-allele model 460 is trained to appropriately analyze the HLA sequence representation 485. In some embodiments, the pan-allele model 460 directly receives, as input, the HLA sequences from the patient 420. Therefore, the HLA sequences from the patient 420 need not undergo a transformation into a representation prior to being input into the pan-allele model 460.

In various embodiments, there may be multiple instances of the pan-allele model 460 in the multi-part presentation model 430. For example, as shown in FIG. 4B, there may be M instances of the pan-allele model 460. Each instance of the pan-allele model 460 may be trained on a random shuffling of training data and therefore, each instance sees different combinations and learns different patterns in the different training data. In various embodiments, M is any of one, two, three, four, five, six, seven, eight, nine, ten, eleven, twelve, thirteen, fourteen, fifteen, sixteen, seventeen, eighteen, nineteen, or twenty instances of the pan-allele model 460. In particular embodiments, M represents 10 instances of the pan-allele model 460.

Referring to the allele-specific model 470, in various embodiments, the allele-specific model 470 may receive, as input, a representation of the peptide sequence 410, which is shown in FIG. 4B as a peptide representation 480. In various embodiments, as shown in FIG. 4B, the peptide presentation inputted into the allele-specific model 470 may be the same peptide representation inputted into the pan-allele model 460. In other embodiments, different peptide representations are generated such that the pan-allele model 460 and the allele-specific model 470 analyze different peptide representations. In some embodiments, the allele-specific model 470 directly receives, as input, the peptide sequence 410. Therefore, the peptide sequence 410 need not undergo a transformation into a peptide representation prior to being input into the allele-specific model 470.

Although not shown in FIG. 4B, in various embodiments, allele-specific models may further receive, as input, additional allele-noninteracting information, an example of which include C-flanking or N-flanking sequences of the peptide sequence 410.

In various embodiments, there may be multiple instances of the allele-specific model 470 in the multi-part presentation model 430. For example, as shown in FIG. 4B, there may be N instances of the allele-specific model 470. In various embodiments, N is any of one, two, three, four, five, six, seven, eight, nine, ten, eleven, twelve, thirteen, fourteen, fifteen, sixteen, seventeen, eighteen, nineteen, or twenty instances of the allele-specific model 470. In particular embodiments, N represents 10 instances of the allele-specific model 470.

As shown in FIG. 4B, the outputs of the allele-specific model 470 are combined with a HLA allele representation 490, which is a representation of the identified HLA alleles 425 expressed by the patient. The combination of the outputs of the allele-specific model 470 and the HLA allele representation 490 can be per-allele presentation likelihoods. In particular embodiments, the HLA allele representation is a one-hot encoding of the identified HLA alleles 425. Thus, the combination of the outputs of the allele-specific model 470 and the HLA allele representation 490 can be per-allele presentation likelihoods of the specific HLA alleles expressed by the patient.

In various embodiments, the per-allele likelihoods determined by the pan-allele model 460 and the per-allele likelihoods determined by the plurality of allele-specific models 470 undergo a transformation, such as a sigmoid transformation as shown in FIG. 4B. Other scaling transformations may be implemented. In various embodiments, the transformation of the per-allele likelihoods is optional and need not be performed.

The per-allele likelihoods determined by the pan-allele model 460 and the per-allele likelihoods determined by the plurality of allele-specific models 470 are combined to generate the final presentation likelihoods 440. In various embodiments, the combination is a statistical combination of the per-allele likelihoods determined by the pan-allele model 460 and the per-allele likelihoods determined by the plurality of allele-specific models 470. Example statistical combinations include an average, median, mode, variance, and standard deviation. In particular embodiments, the final presentation likelihoods 440 represent an average of the per-allele likelihoods determined by the pan-allele model 460 and the per-allele likelihoods determined by the plurality of allele-specific models 470. In various embodiments, the final presentation likelihoods 440 may represent a weighted average of the per-allele likelihoods determined by the pan-allele model 460 and the per-allele likelihoods determined by the plurality of allele-specific models 470. This enables the final presentation likelihoods 440 to more heavily weigh the contribution of one type of model over the type of model.

In various embodiments, regardless of the specific type of multi-part presentation model, all of the multi-part presentation models capture the dependence between independent variables and dependent variables in the training data 170 such that a loss function is minimized. Specifically, the loss function [symbol] (y_{i∈S}, u_{i∈S}; θ) represents discrepancies between values of dependent variables yⁱ_{E}S for one or more data instances S in the training data 170 and the estimated likelihoods u_{i∈S} for the data instances S generated by the presentation model. In one particular implementation referred throughout the remainder of the specification, the loss function (y_{i∈S}, u_{i∈S}; θ) is the negative log likelihood function given by equation (1a) as follows:

$$ℓ ( {y}_{i ∈ S} , {u}_{i ∈ S} ; θ ) = {\sum }_{i ∈ S} ( {y}_{i} log {u}_{i} + ( 1 - {y}_{i} ) log ( 1 - {u}_{i} ) ) . \qquad ( 1 a )$$

However, in practice, another loss function may be used. For example, when predictions are made for the mass spectrometry ion current, the loss function is the mean squared loss given by equation 1b as follows:

$$ℓ ( {y}_{i ∈ S} , {u}_{i ∈ S} ; θ ) = {\sum }_{i ∈ S} ( {‖ {y}_{i} - {u}_{i} ‖}_{2}^{2} ) . \qquad ( 1 b )$$

The multi-part presentation model may be a parametric model in which one or more parameters θ mathematically specify the dependence between the independent variables and dependent variables. Typically, various parameters of parametric-type presentation models that minimize the loss function (y_{i∈S}, u_{i∈S}; θ) are determined through gradient-based numerical optimization algorithms, such as batch gradient algorithms, stochastic gradient algorithms, and the like. Alternatively, the multi-part presentation model may be a non-parametric model in which the model structure is determined from the training data 170 and is not strictly based on a fixed set of parameters.

In various embodiments, the multi-part presentation model achieves a particular performance metric, such as an area under a precision recall curve (PR-AUC) performance metric. In various embodiments, the multi-part presentation model achieves a PR-AUC of at least 0.05, at least 0.06, at least 0.07, at least 0.08, at least 0.09, at least 0.1, 0.11, at least 0.12, at least 0.13, at least 0.14, at least 0.15, at least 0.16, at least 0.17, at least 0.18, at least 0.19, at least 0.20, at least 0.21, at least 0.22, at least 0.23, at least 0.24, at least 0.25, at least 0.26, at least 0.27, at least 0.28, at least 0.29, at least 0.30, at least 0.31, at least 0.32, at least 0.33, at least 0.34, at least 0.35, at least 0.36, at least 0.37, at least 0.38, at least 0.39, at least 0.40, at least 0.41, at least 0.42, at least 0.43, at least 0.44, at least 0.45, at least 0.46, at least 0.47, at least 0.48, at least 0.49, at least 0.50, at least 0.51, at least 0.52, at least 0.53, at least 0.54, at least 0.55, at least 0.56, at least 0.57, at least 0.58, at least 0.59, at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99. In various embodiments, the multi-part presentation model achieves a PR-AUC of at least 0.05. In various embodiments, the multi-part presentation model achieves a PR-AUC of at least 0.10. In various embodiments, the multi-part presentation model achieves a PR-AUC of at least 0.15. In various embodiments, the multi-part presentation model achieves a PR-AUC of at least 0.20. In various embodiments, the multi-part presentation model achieves a PR-AUC of at least 0.25. In various embodiments, the multi-part presentation model achieves a PR-AUC of at least 0.30. In various embodiments, the performance of the multi-part presentation model is measured in relation to presentation of viral epitopes by X number of alleles (e.g., class I or class II HLA alleles). In various embodiments, the X number of alleles include 1 allele, 2 alleles, 3 alleles, 4 alleles, 5 alleles, 6 alleles, 7 alleles, 8 alleles, 9 alleles, 10 alleles, 11 alleles, 12 alleles, 13 alleles, 14 alleles, 15 alleles, 16 alleles, 17 alleles, 18 alleles, 19 alleles, 20 alleles, 21 alleles, 22 alleles, 23 alleles, 24 alleles, 25 alleles, 26 alleles, 27 alleles, 28 alleles, 29 alleles, or 30 alleles. In particular embodiments, the X number of alleles include 5 alleles. In particular embodiments, the X number of alleles include 10 alleles. In particular embodiments, the X number of alleles include 15 alleles. In particular embodiments, the X number of alleles include 20 alleles. In particular embodiments, the X number of alleles include 25 alleles. In particular embodiments, the X number of alleles include 30 alleles.

### VIII.B. Per-Allele Models

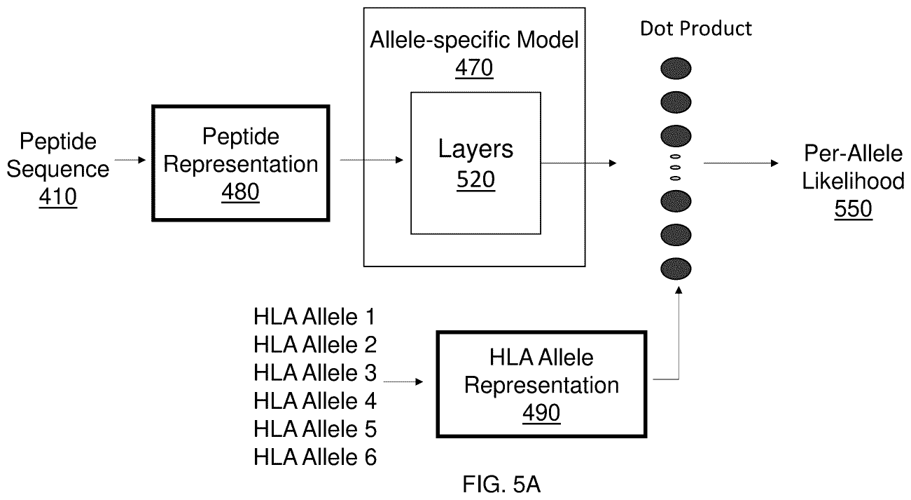

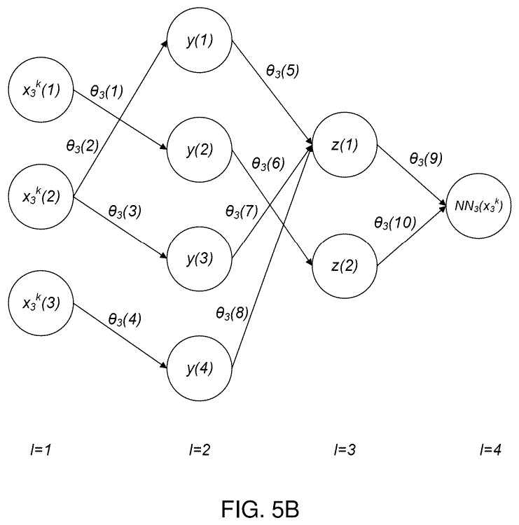

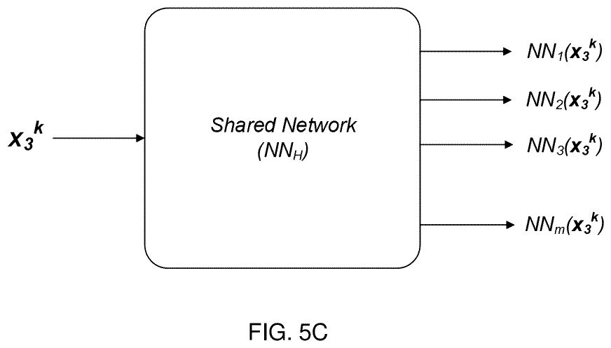

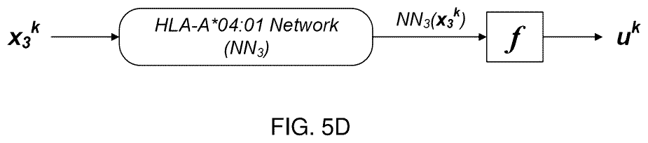

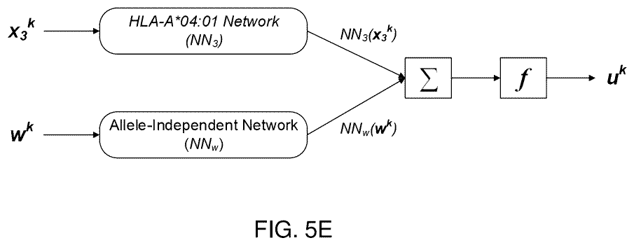

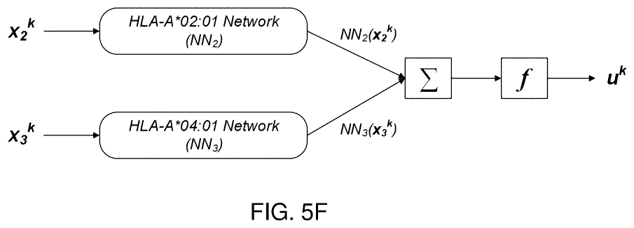

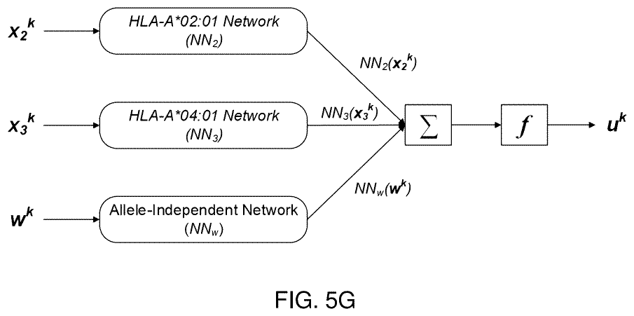

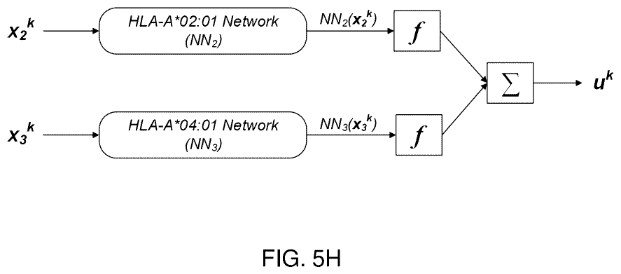

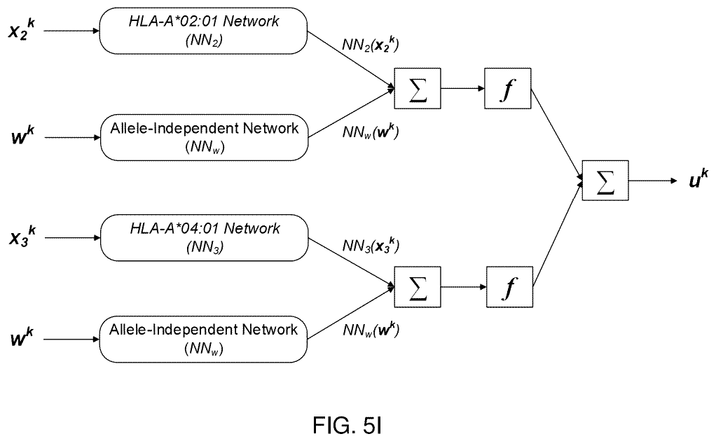

Reference is now made to FIG. 5A, which shows the implementation of an allele-specific model portion of the multi-part presentation model, according to one embodiment. In various embodiments, the allele-specific model 470 may be a neural network. In particular embodiments, the allele-specific model 470 is a multilayer perceptron (MLP). Thus, the allele-specific model 470 may be composed of a plurality of layers 520. As described in further detail herein, the neural network may further include nodes within the layer 520 as well as connections between nodes with associated parameters. In various embodiments, the allele-specific model 470 includes two or more layers 520. In various embodiments, the allele-specific model 470 includes three or more, four or more, five or more, six or more, seven or more, eight or more, nine or more, or ten or more layers 520.

The initial layers of the allele-specific model 470, hereafter referred to as the input layers, receives the values of the peptide representation 480, which is derived from a peptie sequence 410. In various embodiments, the peptide sequence 410 is from an infectious disease-derived peptide. In various embodiments, the peptide sequence 410 is from a human peptide. The values of the peptide representation 480 are propagated through the layers 520 to generate an output value. Here, the output value is combined with the HLA allele representation 490, which is generated based on the identified HLA alleles (e.g., HLA Allele 1 through HLA Allele 6) that are expressed by the patient. As shown in FIG. 5A, the combination of the output of the allele-specific model 470 and the HLA allele representation 490 results in the per-allele likelihood 550 values. Here, the combination may be a dot product between the output of the allele-specific model 470 and the HLA allele representation 490. Therefore, the per-allele likelihood 550 values represent personalized values for the patient based on the HLA alleles expressed. For example, the per allele likelihood values 550 may include six likelihood values corresponding to the six expressed HLA alleles, whereas other likelihood values corresponding to non-expressed HLA alleles equate to zero or near-zero values.

The training module 316 may construct the allele-specific models as per-allele models to predict presentation likelihoods of peptides on an allele-specific basis. In this case, the training module 316 may train the presentation models based on data instances S in the training data 170 generated from cells expressing single MHC alleles.

In one implementation, for a per-allele model, the training module 316 models the estimated presentation likelihood uₖ for peptide p^{k} for a specific allele h by:

$${u}_{k}^{h} = Pr ( {p}^{k} presented ; MHC allele h ) = f ( {g}_{h} ( {x}_{h}^{k} ; {θ}_{h} ) ) , \qquad ( 2 )$$

where xₕ^{k} denotes the encoded allele-interacting variables for peptide p^{k} and corresponding MHC allele h,ƒ(⋅) is any function, and is herein throughout is referred to as a transformation function for convenience of description. Further, gₕ(⋅) is any function, is herein throughout referred to as a dependency function for convenience of description, and generates dependency scores for the allele-interacting variables xₕ^{k} based on a set of parameters θₕ determined for MHC allele h. The values for the set of parameters θₕ for each MHC allele h can be determined by minimizing the loss function with respect to θₕ, where i is each instance in the subset S of training data 170 generated from cells expressing the single MHC allele h.

The output of the dependency function gₕ(xₕ^{k};θₕ) represents a dependency score for the MHC allele h indicating whether the MHC allele h will present the corresponding antigen based on at least the allele interacting features xₕ^{k}, and in particular, based on positions of amino acids of the peptide sequence of peptide p^{k}. For example, the dependency score for the MHC allele h may have a high value if the MHC allele h is likely to present the peptide p^{k}, and may have a low value if presentation is not likely. The transformation function ƒ(⋅) transforms the input, and more specifically, transforms the dependency score generated by gₕ(xₕ^{k};θₕ) in this case, to an appropriate value to indicate the likelihood that the peptide p^{k} will be presented by an MHC allele.

In one particular implementation referred throughout the remainder of the specification, ƒ(⋅) is a function having the range within [0, 1] for an appropriate domain range. In one example, ƒ(⋅) is the expit function given by:

$$f ( z ) = \frac{exp ( z )}{1 + exp ( z )} . \qquad ( 3 )$$

As another example, ƒ(⋅) can also be the hyperbolic tangent function given by:

$$f ( z ) = tanh ( z ) \qquad ( 4 )$$

when the values for the domain z is equal to or greater than 0. Alternatively, when predictions are made for the mass spectrometry ion current that have values outside the range [0, 1], ƒ(⋅) can be any function such as the identity function, the exponential function, the log function, and the like.

Thus, the per-allele likelihood that a peptide sequence p^{k} will be presented by a MHC allele h can be generated by applying the dependency function gₕ(⋅) for the MHC allele h to the encoded version of the peptide sequence p^{k} to generate the corresponding dependency score. The dependency score may be transformed by the transformation function ƒ(⋅) to generate a per-allele likelihood that the peptide sequence p^{k} will be presented by the MHC allele h.

### VIII.B.1 Dependency Functions for Allele Interacting Variables

In one particular implementation referred throughout the specification, the dependency function gₕ(⋅) is an affine function given by:

$${g}_{h} ( {x}_{h}^{i} ; {θ}_{h} ) = {x}_{h}^{i} \cdot {θ}_{h} . \qquad ( 5 )$$

that linearly combines each allele-interacting variable in xₕ^{k} with a corresponding parameter in the set of parameters θₕ determined for the associated MHC allele h.

In another particular implementation referred throughout the specification, the dependency function gₕ(⋅) is a network function given by:

$${g}_{h} ( {x}_{h}^{i} ; {θ}_{h} ) = N {N}_{h} ( {x}_{h}^{i} ; {θ}_{h} ) . \qquad ( 6 )$$

represented by a network model NNₕ(⋅) having a series of nodes arranged in one or more layers. A node may be connected to other nodes through connections each having an associated parameter in the set of parameters θₕ. A value at one particular node may be represented as a sum of the values of nodes connected to the particular node weighted by the associated parameter mapped by an activation function associated with the particular node. In contrast to the affine function, network models are advantageous because the presentation model can incorporate non-linearity and process data having different lengths of amino acid sequences. Specifically, through non-linear modeling, network models can capture interaction between amino acids at different positions in a peptide sequence and how this interaction affects peptide presentation.

In general, network models NNₕ(⋅) may be structured as feed-forward networks, such as artificial neural networks (ANN), convolutional neural networks (CNN), deep neural networks (DNN), and/or recurrent networks, such as long short-term memory networks (LSTM), bi-directional recurrent networks, deep bi-directional recurrent networks, and the like.

In one instance referred throughout the remainder of the specification, each MHC allele in h=1,2, . . . , m is associated with a separate network model, and NNₕ(⋅) denotes the output(s) from a network model associated with MHC allele h.

FIG. 5B illustrates an example network model in association with an MHC allele (e.g., an arbitrary MHC allele h=3). Here, the example network model may include four total layers and may represent the layers 520 of the allele-specific model 470 described in reference to FIG. 5A. As shown in FIG. 5B, the network model NN₃(⋅) for MHC allele h=3 includes three input nodes at layer l=1, four nodes at layer l=2, two nodes at layer l=3, and one output node at layer l=4. The network model NN₃(⋅) is associated with a set of ten parameters θ₃ (1), θ₃ (2), . . . , θ₃ (10). The network model NN₃(⋅) receives input values (individual data instances including encoded polypeptide sequence data and any other training data used) for three allele-interacting variables x₃^{k}(1), x₃^{k}(2), and x₃^{k}(3) for MHC allele h=3 and outputs the value NN₃(x₃^{k}). The network function may also include one or more network models each taking different allele interacting variables as input.

In another instance, the identified MHC alleles h=1, 2, . . . , m are associated with a single network model NN_{H}(⋅), and NNₕ(⋅) denotes one or more outputs of the single network model associated with MHC allele h. In such an instance, the set of parameters θₕ may correspond to a set of parameters for the single network model, and thus, the set of parameters θₕ may be shared by all MHC alleles.

FIG. 5C illustrates an example network model NN_{H}(⋅) shared by MHC alleles h=1,2, . . . , m. As shown in FIG. 5C, the network model NN_{H}(⋅) includes m output nodes each corresponding to an MHC allele. The network model NN₃(⋅) receives the allele-interacting variables x₃^{k} for MHC allele h=3 and outputs m values including the value NN₃(x₃^{k}) corresponding to the MHC allele h=3.

In yet another instance, the dependency function gₕ(⋅) can be expressed as:

$${g}_{h} ( {x}_{h}^{k} ; {θ}_{h} ) = {g}_{h}^{′} ( {x}_{h}^{k} ; {θ}_{h}^{′} ) + {θ}_{h}^{0}$$

where g′ₕ(xₕ^{k};θ′ₕ) is the affine function with a set of parameters θ′ₕ, the network function, or the like, with a bias parameter θₕ⁰ in the set of parameters for allele interacting variables for the MHC allele that represents a baseline probability of presentation for the MHC allele h.

In another implementation, the bias parameter θ′ₕ may be shared according to the gene family of the MHC allele h. That is, the bias parameter θₕ⁰ for MHC allele h may be equal to θ_{gene(h)}⁰, where gene(h) is the gene family of MHC allele h. For example, class I MHC alleles HLA-A*02:01, HLA-A*02:02, and HLA-A*02:03 may be assigned to the gene family of “HLA-A,” and the bias parameter θₕ⁰ for each of these MHC alleles may be shared. As another example, class II MHC alleles HLA-DRB1:10:01, HLA-DRB1:11:01, and HLA-DRB3:01:01 may be assigned to the gene family of “HLA-DRB,” and the bias parameter θₕ⁰ for each of these MHC alleles may be shared.

Returning to equation (2), as an example, the likelihood that peptide p^{k} will be presented by MHC allele h=3, among m=4 different identified MHC alleles using the affine dependency function gₕ(⋅), can be generated by:

uₖ³=ƒ(x₃^{k}·θ₃),

where x₃^{k} are the identified allele-interacting variables for MHC allele h=3, and θ₃ are the set of parameters determined for MHC allele h=3 through loss function minimization.

As another example, the likelihood that peptide p^{k} will be presented by MHC allele h=3, among m=4 different identified MHC alleles using separate network transformation functions gₕ(⋅), can be generated by:

uₖ³=ƒ(NN₃(x₃^{k};θ₃)),

where x₃^{k} are the identified allele-interacting variables for MHC allele h=3, and θ₃ are the set of parameters determined for the network model NN₃(⋅) associated with MHC allele h=3.

FIG. 5D illustrates generating a presentation likelihood for peptide p^{k} in association with MHC allele h=3 using an example network model NN₃(⋅). As shown in FIG. 5D, the network model NN₃(⋅) receives the allele-interacting variables x₃^{k} for MHC allele h=3 and generates the output NN₃(x₃^{k}). The output is mapped by function ƒ(⋅) to generate the estimated presentation likelihood uₖ.

### VIII.B.2. Per-Allele with Allele-Noninteracting Variables

In one implementation, the training module 316 incorporates allele-noninteracting variables and models the estimated presentation likelihood uₖ for peptide p^{k} by:

$${u}_{k}^{h} = Pr ( {p}^{k} presented ) = f ( {g}_{w} ( {w}^{k} ; {θ}_{w} ) + {g}_{h} ( {x}_{h}^{i} ; {θ}_{h} ) ) , \qquad ( 7 )$$

where w^{k} denotes the encoded allele-noninteracting variables for peptide p^{k}, g_{w}(⋅) is a function for the allele-noninteracting variables w^{k} based on a set of parameters θ_{w} determined for the allele-noninteracting variables. Specifically, the values for the set of parameters θₕ for each MHC allele h and the set of parameters θ_{w} for allele-noninteracting variables can be determined by minimizing the loss function with respect to θₕ and θ_{w}, where i is each instance in the subset S of training data 170 generated from cells expressing single MHC alleles.

The output of the dependency function g_{w}(w^{k};θ′_{w}) represents a dependency score for the allele noninteracting variables indicating whether the peptide p^{k} will be presented by one or more MHC alleles based on the impact of allele noninteracting variables. For example, the dependency score for the allele noninteracting variables may have a high value if the peptide p^{k} is associated with a C-terminal flanking sequence that is known to positively impact presentation of the peptide p^{k}, and may have a low value if the peptide p^{k} is associated with a C-terminal flanking sequence that is known to negatively impact presentation of the peptide p^{k}.

According to equation (7), the per-allele likelihood that a peptide sequence p^{k} will be presented by a MHC allele h can be generated by applying the function gₕ(⋅) for the MHC allele h to the encoded version of the peptide sequence p^{k} to generate the corresponding dependency score for allele interacting variables. The function g_{w}(⋅) for the allele noninteracting variables are also applied to the encoded version of the allele noninteracting variables to generate the dependency score for the allele noninteracting variables. Both scores are combined, and the combined score is transformed by the transformation function ƒ(⋅) to generate a per-allele likelihood that the peptide sequence p^{k} will be presented by the MHC allele h.

Alternatively, the training module 316 may include allele-noninteracting variables w^{k} in the prediction by adding the allele-noninteracting variables w^{k} to the allele-interacting variables xₕ^{k} in equation (2). Thus, the presentation likelihood can be given by:

$${u}_{k}^{h} = Pr ( {p}^{k} presented ; allele h ) = f ( {g}_{h} ( [ {x}_{h}^{k} {w}^{k} ] ; {θ}_{h} ) ) . \qquad ( 8 )$$

### VIII.B.3 Dependency Functions for Allele-Noninteracting Variables

Similarly to the dependency function gₕ(⋅) for allele-interacting variables, the dependency function g_{w}(⋅) for allele noninteracting variables may be an affine function or a network function in which a separate network model is associated with allele-noninteracting variables w^{k}.

Specifically, the dependency function g_{w}(⋅) is an affine function given by:

g_{w}(w^{k};θ_{w})=w^{k}·θ_{w}.

that linearly combines the allele-noninteracting variables in w^{k} with a corresponding parameter in the set of parameters θ_{w}.

The dependency function g_{w}(⋅) may also be a network function given by:

g_{w}(w^{k};θ_{w})=NN_{w}(w^{k};θ_{w}).

represented by a network model NN_{H}(⋅) having an associated parameter in the set of parameters θ_{w}. The network function may also include one or more network models each taking different allele noninteracting variables as input.

In another instance, the dependency function g_{w}(⋅) for the allele-noninteracting variables can be given by:

$${g}_{w} ( {w}^{k} ; {θ}_{w} ) = {g}_{w}^{′} ( {w}^{k} ; {θ}_{w}^{′} ) + h ( {m}^{k} ; {θ}_{w}^{m} ) , \qquad ( 9 )$$

where g′_{w}(w^{k};θ′_{w}) is the affine function, the network function with the set of allele noninteracting parameters θ′_{w}, or the like, m^{k} is the mRNA quantification measurement for peptide p^{k}, h(⋅) is a function transforming the quantification measurement, and θ_{w}^{m} is a parameter in the set of parameters for allele noninteracting variables that is combined with the mRNA quantification measurement to generate a dependency score for the mRNA quantification measurement. In one particular embodiment referred throughout the remainder of the specification, h(⋅) is the log function, however in practice h(⋅) may be any one of a variety of different functions.

In yet another instance, the dependency function g_{w}(⋅) for the allele-noninteracting variables can be given by:

$${g}_{w} ( {w}^{k} ; {θ}_{w} ) = {g}_{w}^{′} ( {w}^{k} ; {θ}_{w}^{′} ) + {θ}_{w}^{o} \cdot {o}^{k} , \qquad ( 10 )$$

where g′_{w}(w^{k};θ′_{w}) is the affine function, the network function with the set of allele noninteracting parameters θ′_{w}, or the like, o^{k} is the indicator vector representing proteins and isoforms in the human proteome for peptide p^{k}, and θ_{w}^{o} is a set of parameters in the set of parameters for allele noninteracting variables that is combined with the indicator vector. In one variation, when the dimensionality of o^{k} and the set of parameters θ_{w}^{o} are significantly high, a parameter regularization term, such as λ⋅∥θ_{w}^{o}∥, where ∥⋅∥ represents L1 norm, L2 norm, a combination, or the like, can be added to the loss function when determining the value of the parameters. The optimal value of the hyperparameter X can be determined through appropriate methods.

In yet another instance, the dependency function g_{w}(⋅) for the allele-noninteracting variables can be given by:

$${g}_{w} ( {w}^{k} ; {θ}_{w} ) = {g}_{w}^{′} ( {w}^{k} ; {θ}_{w}^{′} ) + {\sum }_{l = 1}^{L} 𝟙 ( gene ( {p}^{k} ) = l ) \cdot {θ}_{w}^{l} , \qquad ( 11 )$$

where g′_{w}(w^{k};θ′_{w}) is the affine function, the network function with the set of allele noninteracting parameters θ′_{w}, or the like, [symbol] (gene(p^{k}=l)) is the indicator function that equals to 1 if peptide p^{k} is from source gene l as described above in reference to allele noninteracting variables, and θw^{l} is a parameter indicating “antigenicity” of source gene l. In one variation, when L is significantly high, and thus, the number of parameters θ_{w}^{l=1, 2, . . . , L} are significantly high, a parameter regularization term, such as λ⋅∥θ_{w}^{l}∥, where ∥⋅∥ represents L1 norm, L2 norm, a combination, or the like, can be added to the loss function when determining the value of the parameters. The optimal value of the hyperparameter X can be determined through appropriate methods.

In yet another instance, the dependency function g_{w}(⋅) for the allele-noninteracting variables can be given by:

$${g}_{w} ( {w}^{k} ; {θ}_{w} ) = {g}_{w}^{′} ( {w}^{k} ; {θ}_{w}^{′} ) + {\sum }_{m = 1}^{M} {\sum }_{l = 1}^{L} 𝟙 ( gene ( {p}^{k} ) = l , tissue ( {p}^{k} ) = m ) \cdot {θ}_{w}^{l m} , \qquad ( 12 a )$$

where g′_{w}(w^{k};θ′_{w}) is the affine function, the network function with the set of allele noninteracting parameters θ′_{w}, or the like, [symbol] gene(p^{k})=l, tissue(p^{k})=m) is the indicator function that equals to 1 if peptide p^{k} is from source gene l and if peptide p^{k} is from tissue type m as described above in reference to allele noninteracting variables, and θ_{w}^{lm} is a parameter indicating antigenicity of the combination of source gene l and tissue type m. Specifically, the antigenicity of gene l for tissue type m may denote the residual propensity for cells of tissue type m to present peptides from gene l after controlling for RNA expression and peptide sequence context.

In one variation, when L or M is significantly high, and thus, the number of parameters θ_{w}^{lm=1, 2, . . . LM} are significantly high, a parameter regularization term, such as as λ⋅∥θ_{w}^{lm}∥, where ∥⋅∥ represents L1 norm, L2 norm, a combination, or the like, can be added to the loss function when determining the value of the parameters. The optimal value of the hyperparameter λ can be determined through appropriate methods. In another variation, a parameter regularization term can be added to the loss function when determining the value of the parameters, such that the parameters for the same source gene do not significantly differ between tissue types. For example, a penalization term such as:

$$λ \cdot {\sum }_{l = 1}^{L} \sqrt{{\sum }_{m = 1}^{M} {( {θ}_{w}^{l m} - \overline{{θ}_{w}^{l}} )}^{2}}$$

where θ̅_̅{̅w̅}̅^̅{̅l̅}̅, is the average antigenicity across tissue types for source gene l, may penalize the standard deviation of antigenicity across different tissue types in the loss function.

In yet another instance, the dependency function g_{w}(⋅) for the allele-noninteracting variables can be given by:

$${g}_{w} ( {w}^{k} ; {θ}_{w} ) = {g}_{w}^{′} ( {w}^{k} ; {θ}_{w}^{′} ) + {\sum }_{l = 1}^{L} 𝟙 ( gene ( {p}^{k} ) = l ) \cdot {θ}_{w}^{l} + {\sum }_{m = 1}^{M} 𝟙 ( loc ( {p}^{k} ) = m ) \cdot {θ}_{w}^{m} \qquad ( 12 b )$$

where g′_{w}(w^{k};θ′_{w}) is the affine function, the network function with the set of allele noninteracting parameters θ′_{w}, or the like, [symbol] (gene(p^{k}=l)) is the indicator function that equals to 1 if peptide p^{k} is from source gene l as described above in reference to allele noninteracting variables, and θ_{w}^{l} is a parameter indicating “antigenicity” of source gene l, and [symbol] (loc(p^{k}=m)) is the indicator function that equals to 1 if peptide p^{k} is from proteomic location m, and θ_{w}^{m} is a parameter indicating the extent to which proteomic location m is a presentation “hotspot”. In one embodiment, a proteomic location can comprise a block of n adjacent peptides from the same protein, where n is a hyperparameter of the model determined via appropriate methods such as grid-search cross-validation.

In practice, the additional terms of any of equations (9), (10), (11), (12a) and (12b) may be combined to generate the dependency function g_{w}(⋅) for allele noninteracting variables. For example, the term h(⋅) indicating mRNA quantification measurement in equation (9) and the term indicating source gene antigenicity in equations (11), (12a), and (12b) may be summed together along with any other affine or network function to generate the dependency function for allele noninteracting variables.

Returning to equation (7), as an example, the likelihood that peptide p^{k} will be presented by MHC allele h=3, among m=4 different identified MHC alleles using the affine transformation functions gₕ(⋅), g_{w}(⋅), can be generated by:

$${u}_{k}^{3} = f ( {w}^{k} \cdot {θ}_{w} + {x}_{3}^{k} \cdot {θ}_{3} ) ,$$

where w^{k} are the identified allele-noninteracting variables for peptide p^{k}, and θ_{w} are the set of parameters determined for the allele-noninteracting variables.

As another example, the likelihood that peptide p^{k} will be presented by MHC allele h=3, among m=4 different identified MHC alleles using the network transformation functions gₕ(⋅), g_{w}(⋅), can be generated by:

$${u}_{k}^{3} = f ( N {N}_{w} ( {w}^{k} ; {θ}_{w} ) + N {N}_{3} ( {x}_{3}^{k} ; {θ}_{3} ) )$$

where w^{k} are the identified allele-interacting variables for peptide p^{k}, and θ_{w} are the set of parameters determined for allele-noninteracting variables.

FIG. 5E illustrates generating a presentation likelihood for peptide p^{k} in association with MHC allele h=3 using example network models NN₃(⋅) and NN_{w}(⋅). As shown in FIG. 5E, the network model NN₃(⋅) receives the allele-interacting variables x₃^{k} for MHC allele h=3 and generates the output NN₃(x₃^{k}). The network model NN_{W}(⋅) receives the allele-noninteracting variables w^{k} for peptide p^{k} and generates the output NN_{w}(w^{k}). The outputs are combined and mapped by function ƒ(⋅) to generate the estimated presentation likelihood uₖ.

### VIII.C. Multiple-Allele Models

The training module 316 may also construct the presentation models to predict presentation likelihoods of peptides in a multiple-allele setting where two or more MHC alleles are present. In this case, the training module 316 may train the presentation models based on data instances S in the training data 170 generated from cells expressing single MHC alleles, cells expressing multiple MHC alleles, or a combination thereof.

### VIII.C.1. Example 1: Maximum of Per-Allele Models

In one implementation, the training module 316 models the estimated presentation likelihood uₖ for peptide p^{k} in association with a set of multiple MHC alleles H as a function of the presentation likelihoods uₖ^{h∈H} determined for each of the MHC alleles h in the set H determined based on cells expressing single-alleles, as described above in conjunction with equations (2)-(10). Specifically, the presentation likelihood uₖ can be any function of uₖ^{h∈H}. In one implementation, as shown in equations (11), (12a), and (12b), the function is the maximum function, and the presentation likelihood uₖ can be determined as the maximum of the presentation likelihoods for each MHC allele h in the set H.

$${u}_{k} = Pr ( {p}^{k} presented ; alleles H ) = max ( {u}_{k}^{h ∈ H} ) .$$

### VIII.C.2. Example 2.1: Function-of-Sums Models

In one implementation, the training module 316 models the estimated presentation likelihood uₖ for peptide p^{k} by:

$${u}_{k} = Pr ( {p}^{k} presented ) = f ( {\sum }_{h = 1}^{m} {a}_{h}^{k} \cdot {g}_{h} ( {x}_{h}^{k} ; {θ}_{h} ) ) , \qquad ( 13 )$$

where elements aₕ^{k} are 1 for the multiple MHC alleles H associated with peptide sequence p^{k} and xₕ^{k} denotes the encoded allele-interacting variables for peptide p^{k} and the corresponding MHC alleles. The values for the set of parameters θₕ for each MHC allele h can be determined by minimizing the loss function with respect to θₕ, where i is each instance in the subset S of training data 170 generated from cells expressing single MHC alleles and/or cells expressing multiple MHC alleles. The dependency function gₕ may be in the form of any of the dependency functions gₕ introduced above.

According to equation (13), the presentation likelihood that a peptide sequence p^{k} will be presented by one or more MHC alleles h can be generated by applying the dependency function gₕ(⋅) to the encoded version of the peptide sequence p^{k} for each of the MHC alleles H to generate the corresponding score for the allele interacting variables. The scores for each MHC allele h are combined, and transformed by the transformation function ƒ(⋅) to generate the presentation likelihood that peptide sequence p^{k} will be presented by the set of MHC alleles H.

The presentation model of equation (13) is different from the per-allele model of equation (2), in that the number of associated alleles for each peptide p^{k} can be greater than 1. In other words, more than one element in aₕ^{k} can have values of 1 for the multiple MHC alleles H associated with peptide sequence p^{k}.

As an example, the likelihood that peptide p^{k} will be presented by MHC alleles h=2, h=3, among m=4 different identified MHC alleles using the affine transformation functions gₕ(⋅), can be generated by:

$${u}_{k} = f ( {x}_{2}^{k} \cdot {θ}_{2} + {x}_{3}^{k} \cdot {θ}_{3} ) ,$$

where x₂^{k}, x₃^{k} are the identified allele-interacting variables for MHC alleles h=2, h=3, and θ₂, θ₃ are the set of parameters determined for MHC alleles h=2, h=3.

As another example, the likelihood that peptide p^{k} will be presented by MHC alleles h=2, h=3, among m=4 different identified MHC alleles using the network transformation functions gₕ(⋅), g_{w}(⋅), can be generated by:

$${u}_{k} = f ( {NN}_{2} ( {x}_{2}^{k} ; {θ}_{2} ) + {NN}_{3} ( {x}_{3}^{k} ; {θ}_{3} ) ) ,$$

where NN₂(⋅), NN₃(⋅) are the identified network models for MHC alleles h=2, h=3, and θ₂, θ₃ are the set of parameters determined for MHC alleles h=2, h=3.

FIG. 5F illustrates generating a presentation likelihood for peptide p^{k} in association with MHC alleles h=2, h=3 using example network models NN₂(⋅) and NN₃(⋅). As shown in FIG. 5F, the network model NN₂(⋅) receives the allele-interacting variables x₂^{k} for MHC allele h=2 and generates the output NN₂(x₂^{k}) and the network model NN₃(⋅) receives the allele-interacting variables x₃^{k} for MHC allele h=3 and generates the output NN₃(x₃^{k}). The outputs are combined and mapped by function ƒ(⋅) to generate the estimated presentation likelihood uₖ.

### VIII.C.3. Example 2.2: Function-of-Sums Models with Allele-Noninteracting Variables

In one implementation, the training module 316 incorporates allele-noninteracting variables and models the estimated presentation likelihood uₖ for peptide p^{k} by:

$${u}_{k} = Pr ( {p}^{k} presented ) = f ( {g}_{w} ( {w}^{k} ; {θ}_{w} ) + {\sum }_{h = 1}^{m} {a}_{h}^{k} \cdot {g}_{h} ( {x}_{h}^{k} ; {θ}_{h} ) ) , \qquad ( 14 )$$

where w^{k} denotes the encoded allele-noninteracting variables for peptide p^{k}. Specifically, the values for the set of parameters θₕ for each MHC allele h and the set of parameters θ_{w} for allele-noninteracting variables can be determined by minimizing the loss function with respect to θₕ and θ_{w}, where i is each instance in the subset S of training data 170 generated from cells expressing single MHC alleles and/or cells expressing multiple MHC alleles. The dependency function g_{w} may be in the form of any of the dependency functions g_{w} introduced above.

Thus, according to equation (14), the presentation likelihood that a peptide sequence p^{k} will be presented by one or more MHC alleles H can be generated by applying the function gₕ(⋅) to the encoded version of the peptide sequence p^{k} for each of the MHC alleles H to generate the corresponding dependency score for allele interacting variables for each MHC allele h. The function g_{w}(⋅) for the allele noninteracting variables is also applied to the encoded version of the allele noninteracting variables to generate the dependency score for the allele noninteracting variables. The scores are combined, and the combined score is transformed by the transformation function ƒ(⋅) to generate the presentation likelihood that peptide sequence p^{k} will be presented by the MHC alleles H.

In the presentation model of equation (14), the number of associated alleles for each peptide p^{k} can be greater than 1. In other words, more than one element in aₕ^{k} can have values of 1 for the multiple MHC alleles H associated with peptide sequence p^{k}.

As an example, the likelihood that peptide p^{k} will be presented by MHC alleles h=2, h=3, among m=4 different identified MHC alleles using the affine transformation functions gₕ(⋅), g_{w}(⋅), can be generated by:

$${u}_{k} = f ( {w}^{k} \cdot {θ}_{w} + {x}_{2}^{k} \cdot {θ}_{2} + {x}_{3}^{k} \cdot {θ}_{3} ) ,$$

where w^{k} are the identified allele-noninteracting variables for peptide p^{k}, and θ_{w} are the set of parameters determined for the allele-noninteracting variables.

As another example, the likelihood that peptide p^{k} will be presented by MHC alleles h=2, h=3, among m=4 different identified MHC alleles using the network transformation functions gₕ(⋅), g_{w}(⋅), can be generated by:

$${u}_{k} = f ( {NN}_{w} ( {w}^{k} ; {θ}_{w} ) + {NN}_{2} ( {x}_{2}^{k} ; {θ}_{2} ) + {NN}_{3} ( {x}_{3}^{k} ; {θ}_{3} ) )$$

where w^{k} are the identified allele-interacting variables for peptide p^{k}, and θ_{w} are the set of parameters determined for allele-noninteracting variables.

FIG. 5G illustrates generating a presentation likelihood for peptide p^{k} in association with MHC alleles h=2, h=3 using example network models NN₂(⋅), NN₃(⋅), and NN_{H}(⋅). As shown in FIG. 5G, the network model NN₂(⋅) receives the allele-interacting variables x₂^{k} for MHC allele h=2 and generates the output NN₂(x₂^{k}). The network model NN₃(⋅) receives the allele-interacting variables x₃^{k} for MHC allele h=3 and generates the output NN₃(x₃^{k}). The network model NN_{H}(⋅) receives the allele-noninteracting variables w^{k} for peptide p^{k} and generates the output NN_{w}(w^{k}). The outputs are combined and mapped by function ƒ(⋅) to generate the estimated presentation likelihood uₖ.

Alternatively, the training module 316 may include allele-noninteracting variables w^{k} in the prediction by adding the allele-noninteracting variables w^{k} to the allele-interacting variables xₕ^{k} in equation (15). Thus, the presentation likelihood can be given by:

$${u}_{k} = Pr ( {p}^{k} presented ) = f ( {\sum }_{h = 1}^{m} {a}_{h}^{k} \cdot {g}_{h} ( [ {x}_{h}^{k} {w}^{k} ] ; {θ}_{h} ) ) . \qquad ( 15 )$$

### VIII.C.4. Example 3.1: Models Using Implicit Per-Allele Likelihoods

In another implementation, the training module 316 models the estimated presentation likelihood uₖ for peptide p^{k} by:

$${u}_{k} = Pr ( {p}^{k} presented ) = r ( s ( v = [ \begin{matrix}{a}_{1}^{k} \cdot {{u}_{k}^{′}}^{1} ( θ ) & … & {a}_{m}^{k} \cdot {{u}_{k}^{′}}^{m} ( θ )\end{matrix} ] ) ) , \qquad ( 16 )$$

where elements aₕ^{k} are 1 for the multiple MHC alleles h∈H associated with peptide sequence p^{k}, u′ₖ^{h} is an implicit per-allele presentation likelihood for MHC allele h, vector v is a vector in which element vₕ corresponds to aₕ^{k}·u′ₖ^{h}, s(⋅) is a function mapping the elements of v, and r(⋅) is a clipping function that clips the value of the input into a given range. As described below in more detail, s(⋅) may be the summation function or the second-order function, but it is appreciated that in other embodiments, s(⋅) can be any function such as the maximum function. The values for the set of parameters θ for the implicit per-allele likelihoods can be determined by minimizing the loss function with respect to θ, where i is each instance in the subset S of training data 170 generated from cells expressing single MHC alleles and/or cells expressing multiple MHC alleles.

The presentation likelihood in the presentation model of equation (16) is modeled as a function of implicit per-allele presentation likelihoods u′ₖ^{h} that each correspond to the likelihood peptide p^{k} will be presented by an individual MHC allele h. The implicit per-allele likelihood is distinct from the per-allele presentation likelihood in that the parameters for implicit per-allele likelihoods can be learned from multiple allele settings, in which direct association between a presented peptide and the corresponding MHC allele is unknown, in addition to single-allele settings. Thus, in a multiple-allele setting, the presentation model can estimate not only whether peptide p^{k} will be presented by a set of MHC alleles H as a whole, but can also provide individual likelihoods u′ₖ^{h∈H} that indicate which MHC allele h most likely presented peptide p^{k}. An advantage of this is that the presentation model can generate the implicit likelihoods without training data for cells expressing single MHC alleles.

In one particular implementation referred throughout the remainder of the specification, r(⋅) is a function having the range [0, 1]. For example, r(⋅) may be the clip function:

r(z)=min(max(z,0),1),

where the minimum value between z and 1 is chosen as the presentation likelihood uₖ. In another implementation, r(⋅) is the hyperbolic tangent function given by:

r(z)=tanh(z)

when the values for the domain z is equal to or greater than 0.

### VIII.C.5. Example 3.2: Sum-of-Functions Model

In one particular implementation, s(⋅) is a summation function, and the presentation likelihood is given by summing the implicit per-allele presentation likelihoods:

$${u}_{k} = Pr ( {p}^{k} presented ) = r ( {\sum }_{h = 1}^{m} {a}_{h}^{k} \cdot {{u}_{k}^{′}}^{h} θ ) ) . \qquad ( 17 )$$

In one implementation, the implicit per-allele presentation likelihood for MHC allele h is generated by:

$${{u}_{k}^{′}}^{h} = f ( {g}_{h} ( {x}_{h}^{k} ; {θ}_{h} ) ) ,$$

such that the presentation likelihood is estimated by:

$${u}_{k} = Pr ( {p}^{k} presented ) = r ( {\sum }_{h = 1}^{m} {a}_{h}^{k} \cdot f ( {g}_{h} ( {x}_{h}^{k} ; {θ}_{h} ) ) ) . \qquad ( 19 )$$

According to equation (19), the presentation likelihood that a peptide sequence p^{k} will be presented by one or more MHC alleles H can be generated by applying the function gₕ(⋅) to the encoded version of the peptide sequence p^{k} for each of the MHC alleles H to generate the corresponding dependency score for allele interacting variables. Each dependency score is first transformed by the function ƒ(⋅) to generate implicit per-allele presentation likelihoods u′ₖ^{h}. The per-allele likelihoods u′ₖ^{h} are combined, and the clipping function may be applied to the combined likelihoods to clip the values into a range [0, 1] to generate the presentation likelihood that peptide sequence p^{k} will be presented by the set of MHC alleles H. The dependency function gₕ may be in the form of any of the dependency functions gₕ introduced above.

As an example, the likelihood that peptide p^{k} will be presented by MHC alleles h=2, h=3, among m=4 different identified MHC alleles using the affine transformation functions gₕ(⋅), can be generated by:

$${u}_{k} = r ( f ( {x}_{2}^{k} \cdot {θ}_{2} ) + f ( {x}_{3}^{k} \cdot {θ}_{3} ) ) ,$$

where x₂^{k}, x₃^{k} are the identified allele-interacting variables for MHC alleles h=2, h=3, and θ₂, θ₃ are the set of parameters determined for MHC alleles h=2, h=3.

As another example, the likelihood that peptide p^{k} will be presented by MHC alleles h=2, h=3, among m=4 different identified MHC alleles using the network transformation functions gₕ(⋅), g_{w}(⋅), can be generated by:

$${u}_{k} = r ( f ( {NN}_{2} ( {x}_{2}^{k} ; {θ}_{2} ) ) + f ( {NN}_{3} ( {x}_{3}^{k} ; {θ}_{3} ) ) ) ,$$

where NN₂(⋅), NN₃(⋅) are the identified network models for MHC alleles h=2, h=3, and θ₂, θ₃ are the set of parameters determined for MHC alleles h=2, h=3.

FIG. 5H illustrates generating a presentation likelihood for peptide p^{k} in association with MHC alleles h=2, h=3 using example network models NN₂(⋅) and NN₃(⋅). As shown in FIG. 5H, the network model NN₂(⋅) receives the allele-interacting variables x₂^{k} for MHC allele h=2 and generates the output NN₂(x₂^{k}) and the network model NN₃(⋅) receives the allele-interacting variables x₃^{k} for MHC allele h=3 and generates the output NN₃(x₃^{k}). Each output is mapped by function ƒ(⋅) and combined to generate the estimated presentation likelihood uₖ.

In another implementation, when the predictions are made for the log of mass spectrometry ion currents, r(⋅) is the log function and ƒ(⋅) is the exponential function.

### VIII.C.6. Example 3.3: Sum-of-Functions Models with Allele-Noninteracting Variables

In one implementation, the implicit per-allele presentation likelihood for MHC allele h is generated by:

$${{u}_{k}^{′}}^{h} = f ( {g}_{h} ( {x}_{h}^{k} ; {θ}_{h} ) + {g}_{w} ( {w}^{k} ; {θ}_{w} ) ) , \qquad ( 20 )$$

such that the presentation likelihood is generated by:

$${u}_{k} = Pr ( {p}^{k} presented ) = r ( ( {\sum }_{h = 1}^{m} {a}_{h}^{k} \cdot f ( {g}_{w} ( {w}^{k} ; {θ}_{w} ) + {g}_{h} ( {x}_{h}^{k} ; {θ}_{h} ) ) ) , \qquad ( 21 )$$

to incorporate the impact of allele noninteracting variables on peptide presentation.

According to equation (21), the presentation likelihood that a peptide sequence p^{k} will be presented by one or more MHC alleles H can be generated by applying the function gₕ(⋅) to the encoded version of the peptide sequence p^{k} for each of the MHC alleles H to generate the corresponding dependency score for allele interacting variables for each MHC allele h. The function g_{w}(⋅) for the allele noninteracting variables is also applied to the encoded version of the allele noninteracting variables to generate the dependency score for the allele noninteracting variables. The score for the allele noninteracting variables are combined to each of the dependency scores for the allele interacting variables. Each of the combined scores are transformed by the function ƒ(⋅) to generate the implicit per-allele presentation likelihoods. The implicit likelihoods are combined, and the clipping function may be applied to the combined outputs to clip the values into a range [0,1] to generate the presentation likelihood that peptide sequence p^{k} will be presented by the MHC alleles H. The dependency function g_{w} may be in the form of any of the dependency functions g_{w} introduced above.

As an example, the likelihood that peptide p^{k} will be presented by MHC alleles h=2, h=3, among m=4 different identified MHC alleles using the affine transformation functions gₕ(⋅), g_{w}(⋅), can be generated by:

$${u}_{k} = r ( f ( {w}^{k} \cdot {θ}_{w} + {x}_{2}^{k} \cdot {θ}_{2} ) + f ( {w}^{k} \cdot {θ}_{w} + {x}_{3}^{k} \cdot {θ}_{3} ) ) ,$$

where w^{k} are the identified allele-noninteracting variables for peptide p^{k}, and θ_{w} are the set of parameters determined for the allele-noninteracting variables.

As another example, the likelihood that peptide p^{k} will be presented by MHC alleles h=2, h=3, among m=4 different identified MHC alleles using the network transformation functions gₕ(⋅), g_{w}(⋅), can be generated by:

$${u}_{k} = r ( f ( N {N}_{w} ( {w}^{k} ; {θ}_{w} ) + N {N}_{2} ( {x}_{2}^{k} ; {θ}_{2} ) ) + f ( N {N}_{w} ( {w}^{k} ; {θ}_{w} ) + N {N}_{3} ( {x}_{3}^{k} ; {θ}_{3} ) ) )$$

where w^{k} are the identified allele-interacting variables for peptide p^{k}, and θ_{w} are the set of parameters determined for allele-noninteracting variables.

FIG. 5I illustrates generating a presentation likelihood for peptide p^{k} in association with MHC alleles h=2, h=3 using example network models NN₂(⋅), NN₃(⋅), and NN_{H}(⋅). As shown in FIG. 5I, the network model NN₂(⋅) receives the allele-interacting variables x₂^{k} for MHC allele h=2 and generates the output NN₂(x₂^{k}). The network model NN_{w}(⋅) receives the allele-noninteracting variables w^{k} for peptide p^{k} and generates the output NN_{w}(w^{k}). The outputs are combined and mapped by function ƒ(⋅). The network model NN₃(⋅) receives the allele-interacting variables x₃^{k} for MHC allele h=3 and generates the output NN₃(x₃^{k}), which is again combined with the output NN_{w}(w^{k}) of the same network model NN_{w}(⋅) and mapped by function ƒ(⋅). Both outputs are combined to generate the estimated presentation likelihood uₖ.

In another implementation, the implicit per-allele presentation likelihood for MHC allele h is generated by:

$${u}_{k}^{′ h} = f ( {g}_{h} ( [ {x}_{h}^{k} {w}^{k} ] ; {θ}_{h} ) ) . \qquad ( 22 )$$

such that the presentation likelihood is generated by:

$${u}_{k} = Pr ( {p}^{k} presented ) = r ( ( {\sum }_{h = 1}^{m} {a}_{h}^{k} \cdot f ( {g}_{h} [ {x}_{h}^{k} {w}^{k} ] ; {θ}_{h} ) ) ) ,$$

### VIII.C.7. Example 4: Second Order Models

In one implementation, , s(⋅) is a second-order function, and the estimated presentation likelihood uₖ for peptide p^{k} is given by:

$${u}_{k} = Pr ( {p}^{k} presented ) = {\sum }_{h = 1}^{m} {a}_{h}^{k} \cdot {u}_{k}^{′ h} ( θ ) - {\sum }_{h = 1}^{m} {\sum }_{j < h} {a}_{h}^{k} \cdot {a}_{j}^{k} \cdot {u}_{k}^{′ h} ( θ ) \cdot {u}_{k}^{′ j} ( θ ) \qquad ( 23 )$$

where elements u′ₖ^{h} are the implicit per-allele presentation likelihood for MHC allele h. The values for the set of parameters θ for the implicit per-allele likelihoods can be determined by minimizing the loss function with respect to θ, where i is each instance in the subset S of training data 170 generated from cells expressing single MHC alleles and/or cells expressing multiple MHC alleles. The implicit per-allele presentation likelihoods may be in any form shown in equations (18), (20), and (22) described above.

In one aspect, the model of equation (23) may imply that there exists a possibility peptide p^{k} will be presented by two MHC alleles simultaneously, in which the presentation by two HLA alleles is statistically independent.

According to equation (23), the presentation likelihood that a peptide sequence p^{k} will be presented by one or more MHC alleles H can be generated by combining the implicit per-allele presentation likelihoods and subtracting the likelihood that each pair of MHC alleles will simultaneously present the peptide p^{k} from the summation to generate the presentation likelihood that peptide sequence p^{k} will be presented by the MHC alleles H.

As an example, the likelihood that peptide p^{k} will be presented by HLA alleles h=2, h=3, among m=4 different identified HLA alleles using the affine transformation functions gₕ(⋅), can be generated by:

$${u}_{k} = f ( {x}_{2}^{k} \cdot {θ}_{2} ) + f ( {x}_{3}^{k} \cdot {θ}_{3} ) - f ( {x}_{2}^{k} \cdot {θ}_{2} ) \cdot f ( {x}_{3}^{k} \cdot {θ}_{3} ) ,$$

where x₂^{k}, x₃^{k} are the identified allele-interacting variables for HLA alleles h=2, h=3, and θ₂, θ₃ are the set of parameters determined for HLA alleles h=2, h=3.

As another example, the likelihood that peptide p^{k} will be presented by HLA alleles h=2, h=3, among m=4 different identified HLA alleles using the network transformation functions gₕ(⋅), g_{w}(⋅), can be generated by:

$${u}_{k} = f ( N {N}_{2} ( {x}_{2}^{k} ; {θ}_{2} ) ) + f ( N {N}_{3} ( {x}_{3}^{k} ; {θ}_{3} ) ) - f ( N {N}_{2} ( {x}_{2}^{k} ; {θ}_{2} ) ) \cdot f ( N {N}_{3} ( {x}_{3}^{k} ; {θ}_{3} ) ) ,$$

where NN₂(⋅), NN₃(⋅) are the identified network models for HLA alleles h=2, h=3, and θ₂, θ₃ are the set of parameters determined for HLA alleles h=2, h=3.

### VIII.D. Pan-Allele Models

In contrast to the per-allele model, a pan-allele model (e.g., pan-allele model 460 described in FIG. 4B) is a model that is capable of predicting presentation likelihoods of peptides on a pan-allele basis. Specifically, unlike the per-allele model that is capable of predicting the probability that peptides will be presented by one or more known MHC alleles that have been previously used to train the per-allele model, the pan-allele model is a presentation model that is capable of predicting the probability that a peptide will be presented by any MHC allele—including unknown MHC alleles that the model has not previously encountered during training.

Briefly, the pan-allele model is trained by the training module 316. Similar to the training of the per-allele model, the training module 316 may train the pan-allele presentation model based on data instances S in the training data 170 generated from cells expressing single MHC alleles, cells expressing multiple MHC alleles, or a combination thereof. However rather than training the pan-allele presentation model using a particular MHC allele or a particular set of MHC alleles a^{k}ₕ, the training module 316 trains the pan-allele presentation model using all MHC allele peptide sequences dₕ available in the training data 170. Specifically, the training module 316 trains the pan-allele presentation model based on positions of amino acids of the MHC alleles available in the training data 170.

After the pan-allele model has been trained, when a peptide sequence and known or unknown MHC allele peptide sequence are input into the model to determine the probability that the known or unknown MHC allele will present the peptide, the model is able to accurately predict this probability by using information learned during training with similar MHC allele peptide sequences. For example, a pan-allele model trained using training data 170 that does not contain any occurrences of the A*02:07 allele may still accurately predict the presentation of peptides by the A*02:07 allele by drawing upon information learned during training with similar alleles (e.g., alleles in the A*02 gene family). In this way, a single presentation pan-allele model can predict presentation likelihoods of a peptide on any MHC allele.

### VIII.D.2. Advantages of Pan-Allele Models

The principle advantage of the pan-allele presentation model is that the pan-allele presentation model has greater versatility than the per-allele presentation model. As noted above, a per-allele model is capable of predicting the probability that a peptide will be presented by one or more identified MHC alleles that were used to train the per-allele model. In other words, the per-allele model is associated with a limited set of one or more known MHC alleles.

Therefore, given a sample containing a particular set of one or more MHC alleles, to determine the probability that a peptide is presented by the particular set of MHC alleles, a per-allele model that was trained using that particular set of MHC alleles is selected for use. In other words, when relying on per-allele models to predict the probability that a peptide will be presented by an MHC allele, predictions can be made only for MHC alleles that have appeared in the training data 170. Because a large number of MHC alleles exist (particularly for minor variations within the same gene family), a very large quantity of training samples would be required to train per-allele presentation models to be equipped make peptide presentation predictions for all MHC alleles.

In contrast, the pan-allele model is not limited to making predictions for a particular set of one or more MHC alleles on which it was trained. Instead, during use, the pan-allele model is able to accurately predict the probability that a previously-seen and/or a previously-unseen MHC allele will present a given peptide by using information learned during training with similar MHC allele peptide sequences. As a result, the pan-allele model is not associated with a particular set of one or more MHC alleles, and is capable of predicting the probability that a peptide will be presented by any MHC allele. This versatility of the pan-allele model means that a single model can be used to predict the likelihood that any peptide will be presented by any MHC allele. Therefore, use of the pan-allele model reduces the amount of training data required to maximize both individual HLA coverage and population HLA coverage.

### VIII.D.3. Use of Pan-Allele Models

Briefly, when using the pan-allele model to predict the likelihood that a peptide will be presented by a single MHC allele, one set of inputs is provided to the pan-allele model as described in detail below, and the pan-allele model generates a single output. On the other hand, when using the pan-allele model to predict the likelihoods that a peptide will be presented by multiple MHC alleles, the pan-allele model is used iteratively for each MHC allele of the multiple MHC alleles. Specifically, when using the pan-allele model to predict the likelihoods that a peptide will be presented by multiple MHC alleles, a first set of inputs associated with a first MHC allele of the multiple MHC alleles is provided to the pan-allele model, and the pan-allele model generates a first output for the first MHC allele. Then, a second set of inputs associated with a second MHC allele of the multiple MHC alleles is provided to the pan-allele model, and the pan-allele model generates a second output for the second MHC allele. This process is performed iteratively for each MHC allele of the multiple MHC alleles. Finally, the outputs generated by the pan-allele model for each MHC allele of the multiple MHC alleles are combined to generate a single probability that the multiple MHC alleles present the given peptide.

### VIII.D.4. Overview of Pan-Allele Models

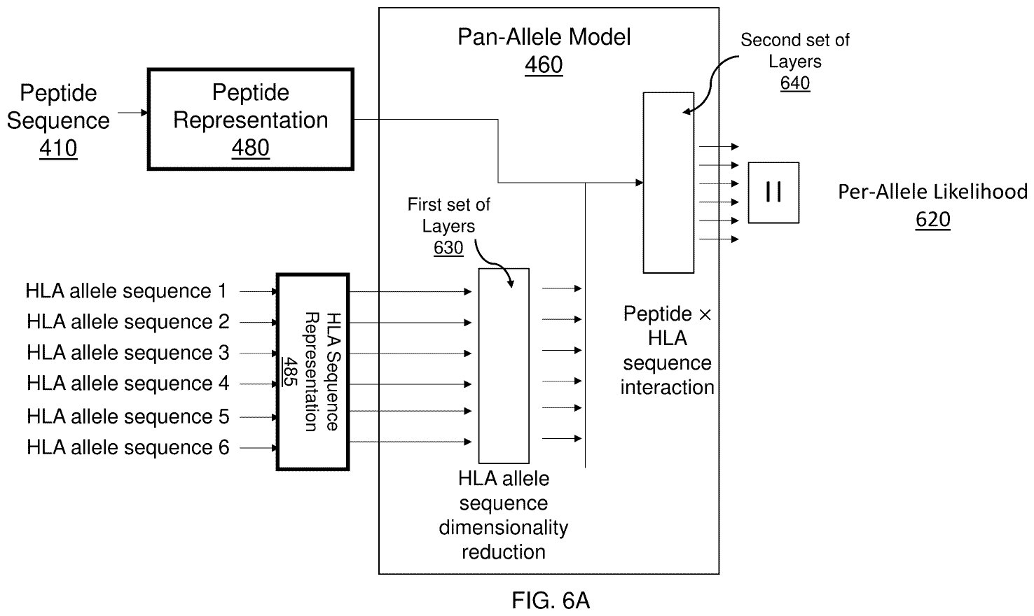

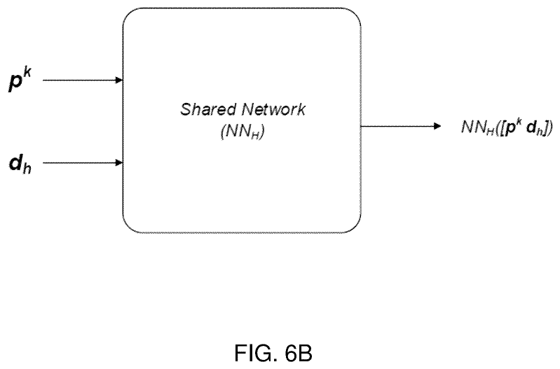

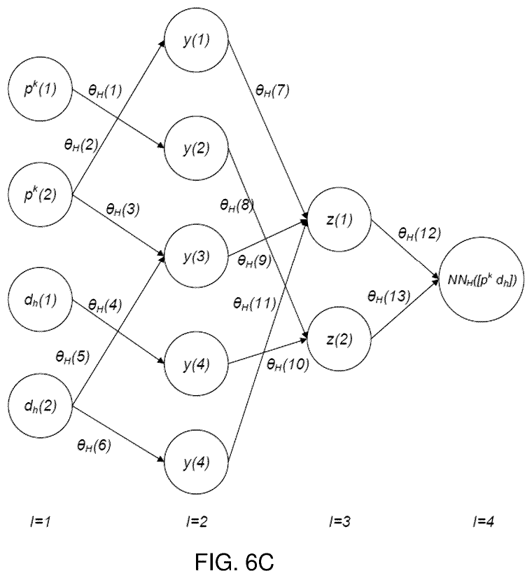

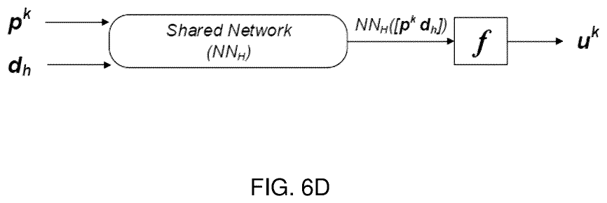

Reference is now made to FIG. 6A, which shows the implementation of a pan-allele model portion 460 of the multi-part presentation model, according to one embodiment. As discussed herein, a multi-part presentation model may include multiple instances of the pan-allele model portion 460 (e.g., 10 instances of the pan-allele model portion 460). In various embodiments, the pan-allele model 460 is a neural network. In various embodiments, as shown in FIG. 6A, the pan-allele model 460 includes two sets of layers (e.g., first set of layers 630 and second set of layers 640). Generally, each of the first set of layers 630 and the second set of layers 640 may be composed of a plurality of layers, nodes within the layers, as well as connections between nodes with associated parameters. In various embodiments, each of the first set of layers 630 and the second set of layers 640 includes two or more layers. In various embodiments, each of the first set of layers 630 and the second set of layers 640 includes three or more, four or more, five or more, six or more, seven or more, eight or more, nine or more, or ten or more layers. In particular embodiments, each of the first set of layers 630 and the second set of layers 640 is a multilayer perceptron (MLP). Generally, the first set of layers 630 and the second set of layers 640 perform different functions, as described in further detail herein.

The first set of layers 630 receives, as input, HLA sequence representation 485, which may be a representation of the sequences of the individual HLA alleles expressed by the patient. In various embodiments, the sequences of the individual HLA alleles undergo encoding (e.g., one-hot encoding scheme) to generate the HLA sequence representation 485. The first set of layers 630 dimensionally reduces the HLA sequence representation 485 to a new representation of lower dimensionality. For example, the new representation of lower dimensionality may retain meaningful properties of the HLA sequence representation 485. In various embodiments, to dimensionally reduce the HLA sequence representation 485, the first set of layers 630 includes an input layer with a greater number of nodes than the number of nodes in the terminal layer.

As shown in FIG. 6A, the second set of layers 640 receives, as input, the peptide representation 480 derived from a peptide sequence 410. In various embodiments, the peptide sequence 410 is from an infectious disease-derived peptide. In various embodiments, the peptide sequence 410 is from a human peptide. Furthermore, the second set of layers 640 may receive, as input, the output of the first set of layers 630. As discussed above, the output of the first set of layers 630 is a dimensionally reduced representation of the HLA allele sequences. The second set of layers 640 models the interactions between the infectious disease-derived peptides and the HLA allele sequences to determine whether the HLA sequences are likely to present the peptide sequences. The output of the second set of layers 640 are per-allele likelihoods 620 (e.g., a likelihood of presentation of the peptide sequence 410 for each of the six HLA alleles expressed by the patient). As discussed above in reference to FIG. 4B, the per-allele likelihoods of the pan-allele model 460 can be transformed and combined to generate the final presentation likelihoods 440.

In one implementation, a pan-allele model is used to estimate the presentation likelihood uₖ for peptide p^{k} for a allele h. In some embodiments, the pan-allele model is represented by the equation:

$${u}_{k}^{h} = Pr ( {p}^{k} presented ; MHC allele h ) = f ( {g}_{H} ( [ {p}^{k} {d}_{h} ] ; {θ}_{H} ) ) , \qquad ( 24 )$$

where p^{k} denotes the peptide sequence, dₕ denotes the peptide sequence of MHC allele h,ƒ(⋅) is any transformation function, and g_{H}(⋅) is any dependency function. The pan-allele model generates dependency scores for the peptide sequence p^{k} and the MHC allele peptide sequence dₕ based on a set of shared parameters θ_{H} determined for all MHC alleles. The values of the set of shared parameters θ_{H} are learned during training of the pan-allele model.

The output of the dependency function g_{H}([p^{k}dₕ];θ_{H}) represents a dependency score for the MHC allele h indicating whether the MHC allele h will present the peptide p^{k} based on at least the positions of amino acids of the peptide sequence p^{k} and the positions of amino acids of the MHC allele peptide sequence dₕ. For example, the dependency score for the MHC allele h may have a high value if the MHC allele h is likely to present the peptide p^{k}given an input MHC allele peptide sequence dₕ, and may have a low value if presentation is not likely. The transformation function ƒ(⋅) transforms the input, and more specifically, transforms the dependency score generated by g_{H}([p^{k} dₕ];θ_{H}) in this case, to an appropriate value to indicate the likelihood that the peptide p^{k} will be presented by the MHC allele h.

In one particular implementation referred to throughout the remainder of the specification, ƒ(⋅) is a function having the range within [0, 1] for an appropriate domain range. In one example, ƒ(⋅) is the expit function. As another example, ƒ(⋅) can also be the hyperbolic tangent function when the values for the domain z is equal to or greater than 0. Alternatively, when predictions are made for the mass spectrometry ion current that have values outside the range [0, 1],ƒ(⋅) can be any function such as the identity function, the exponential function, the log function, and the like.

Thus, the likelihood that a peptide sequence p^{k} will be presented by a MHC allele h can be generated by applying the dependency function g_{H}(⋅) to the encoded version of the peptide sequence p^{k} and to the encoded version of the MCH allele peptide sequence dₕ to generate the corresponding dependency score. The dependency score may be transformed by the transformation function ƒ(⋅) to generate a likelihood that the peptide sequence p^{k} will be presented by the MHC allele h.

### VIII.D.5. Dependency Functions for Allele-Interacting Variables

In one particular implementation referred to throughout the specification, the dependency function g_{H}(⋅) is an affine function given by:

$${g}_{H} ( [ {p}^{k} {d}_{h} ] ; {θ}_{H} ) = {\sum }_{i = 1}^{{n}_{pep}} {\sum }_{j = 1}^{{n}_{M H C}} {\sum }_{k = 1}^{20} {\sum }_{l = 1}^{20} 1 [ {p}_{i}^{k} = k ] 1 [ {d}_{h j} = l ] {θ}_{H , i j k l} + α , \qquad ( 25 )$$

where α is an intercept, pᵢ^{k} denotes the residue at position i of peptide p^{k}, dₕⱼ denotes the residue at position j of MHC allele h, 1[ ] denotes an indicator variable whose value is 1 if the condition inside the brackets is true and 0 otherwise, pᵢ^{k}=k is true if the amino acid at position i of peptide p^{k} is amino acid k and false otherwise, dₕⱼ=l is true if the amino acid at position j of MHC allele h is amino acid l and false otherwise, nₚₑₚ denotes the length of peptides modeled, n_{MHC} denotes the number of MHC residues considered in the model, and θ_{H,ijkl} is a coefficient describing the contribution of having residue k at position i of the peptide and residue l at position j of the MHC allele to the likelihood of presentation. This is a linear model in the one hot-encoded peptide sequence and the one hot-encoded MHC allele sequence, with peptide-residue-by-MHC-residue interactions for all peptide residues and MHC allele residues.

In another particular implementation referred to throughout the specification, the dependency function g_{H}(⋅) is a network function given by:

$${g}_{H} ( [ {p}^{k} {d}_{h} ] ; {θ}_{H} ) = N {N}_{H} ( [ {p}^{k} {d}_{h} ] ; {θ}_{H} ) \qquad ( 26 )$$

represented by a network model NN_{H}(⋅) having a series of nodes arranged in one or more layers. A node may be connected to other nodes through connections each having an associated parameter in the set of parameters θ_{H}. A value at one particular node may be represented as a sum of the values of nodes connected to the particular node weighted by the associated parameter mapped by an activation function associated with the particular node. In contrast to the affine function, network models are advantageous because the presentation model can incorporate non-linearity and process data having different lengths of amino acid sequences. Specifically, through non-linear modeling, network models can capture interaction between amino acids at different positions in a peptide sequence, as well as interaction between amino acids at different positions in a MHC allele peptide sequence, and how these interactions affects peptide presentation.

In general, network models NN_{H}(⋅) may be structured as feed-forward networks, such as artificial neural networks (ANN), convolutional neural networks (CNN), deep neural networks (DNN), and/or recurrent networks, such as long short-term memory networks (LSTM), bi-directional recurrent networks, deep bi-directional recurrent networks, and the like.

In one instance, the single network model NN_{H}(⋅) may be a network model that outputs a dependency score given an encoded peptide sequence p^{k} and an encoded protein sequence dₕ of an MHC allele h. In such an instance, the set of parameters θ_{H} may correspond to a set of parameters for the single network model, and thus, the set of parameters θ_{H} may be shared by all MHC alleles. Thus, in such an instance, NN_{H}(⋅) may denote the output of the single network model NN_{H}(⋅) given any inputs [p^{k}dₕ] to the single network model. As discussed above, such a network model is advantageous because peptide presentation probabilities for MHC alleles that were unknown in the training data can be predicted just by identification of the MHC alleles' protein sequences.

FIG. 6B illustrates an example network model NN_{H}(⋅) shared by MHC alleles. As shown in FIG. 6B, the network model NN_{H}(⋅) receives the peptide sequence p^{k} and protein sequence dₕ of an MHC allele h as input, and outputs a dependency score NN_{H}([p^{k}dₕ]) corresponding to the MHC allele h.

FIG. 6C illustrates an example network model NN_{H}(⋅). Here, the example network model may represent the second set of layers 640 of the pan-allele model 460 shown in FIG. 6A. As shown in FIG. 6C, the network model NN_{H}(⋅) includes four input nodes at layer l=1, five nodes at layer l=2, two nodes at layer l=3, and one output node at layer l=4. In alternative embodiments, the network model NN_{H}(⋅) may contain any number of layers, and each layer may contain any number of nodes. The network model NN_{H}(⋅) is associated with a set of thirteen nonzero parameters θ_{H}(1), θ_{H}(2), . . . , θ_{H}(13). These parameters serve to transform the values that are propagated from node to node, through the network model.

As shown in FIG. 6C, the four input nodes at layer l=1 of the network model NN_{H}(⋅) receive input values including encoded polypeptide sequence data and encoded MHC allele peptide sequence data. The encoded polypeptide sequence data contains the amino acid sequence for a peptide, and the encoded MHC allele peptide sequence data contains the amino acid sequence for an MHC allele that may (or may not) present the peptide. In certain embodiments, once input into the network model NN_{H}(⋅) via the input nodes at layer l=1, the encoded polypeptide sequence is concatenated to the front of the encoded MHC allele peptide sequence within a layer of the network model NN_{H}(⋅). These input values are then propagated through the network model NN_{H}(⋅) according to the values of the parameters. In some embodiments, the layers of the network model NN_{H}(⋅) include two fully-connected dense network layers. In further embodiments, the first layer of these two fully-connected dense network layers comprises between 64-128 nodes with a rectified linear unit activation function. In even further embodiments, the second layer of these two fully-connected dense network layers comprises a single node with a linear output. In such embodiments, this single node may be the output node of the network model NN_{H}(⋅). Finally, the network model NN_{H}(⋅) outputs the value NN_{H}([p^{k} dₕ]). This output represents a dependency score for the MHC allele h indicating whether the MHC allele h will present the peptide sequence p^{k}. The network function may also include one or more network models each taking different allele-interacting variables (e.g., peptide sequences) as input.

In yet another instance, the dependency function g_{H}(⋅) can be expressed as:

$${g}_{H} ( [ {p}^{k} {d}_{h} ] ; {θ}_{H} ) = {g}_{H}^{′} ( [ {p}^{k} {d}_{h} ] ; {θ}_{H}^{′} ) + {θ}_{H}^{0}$$

where g′_{H}([p^{k} dₕ];θ′_{H}) is the affine function with a set of parameters θ′_{H}, the network function, or the like, with a bias parameter θ_{H}⁰ in the set of shared parameters θ_{H} for allele-interacting variables that represents a baseline probability of presentation for any MHC allele.

In another implementation, the bias parameter θ_{H}⁰ may be shared according to the gene family of the MHC allele h. That is, the bias parameter θ_{H}⁰ for MHC allele h may be equal to θ_{gene(h)}⁰, where gene(h) is the gene family of MHC allele h. For example, class I MHC alleles HLA-A*02:01, HLA-A*02:02, and HLA-A*02:03 may be assigned to the gene family of “HLA-A,” and the bias parameter θ_{H}⁰ for each of these MHC alleles may be shared. As another example, class II MHC alleles HLA-DRB1:10:01, HLA-DRB1:11:01, and HLA-DRB3:01:01 may be assigned to the gene family of “HLA-DRB,” and the bias parameter OHI for each of these MHC alleles may be shared. As discussed above, gene family may be one of the allele-interacting variables associated with an MHC allele h.

Returning to equation (23), as an example, the likelihood that peptide p^{k} will be presented by MHC allele h, using the affine dependency function g_{H}(⋅), can be generated by:

$${u}_{k}^{h} = f ( {\sum }_{i = 1}^{{n}_{pep}} {\sum }_{j = 1}^{{n}_{M H C}} {\sum }_{k = 1}^{20} {\sum }_{l = 1}^{20} 1 [ {p}_{i}^{k} = k ] 1 [ {d}_{h j} = l ] {θ}_{H , ijkl} + α ) ,$$

where α is an intercept, p^{k} denotes the residue at position i of peptide p^{k}, dₕⱼ denotes the residue at position j of MHC allele h, l[ ] denotes an indicator variable whose value is 1 if the condition inside the brackets is true and 0 otherwise, pᵢ^{k}=k is true if the amino acid at position i of peptide p^{k}is amino acid k and false otherwise, dₕⱼ=l is true if the amino acid at position j of MHC allele h is amino acid l and false otherwise, nₚₑₚ denotes the length of peptides modeled, n_{MHC} denotes the number of MHC residues considered in the model, and θ_{H,ijkl} is a coefficient describing the contribution of having residue k at position i of the peptide and residue l at position j of the MHC allele to the likelihood of presentation. This is a linear model in the one hot-encoded peptide sequence and the one hot-encoded MHC allele sequence, with peptide-residue-by-MHC-residue interactions for all peptide residues and MHC allele residues.

As another example, the likelihood that peptide p^{k} will be presented by an MHC allele h, using the network transformation function g_{H}(⋅), can be generated by:

uₖ^{h}=ƒ(NN_{H}([p^{k}dₕ];θ_{H})),

where p^{k} denotes the peptide sequence, dₕ denotes the peptide sequence of MHC allele h, and θ_{H} is the set of parameters determined for the network model NN_{H}(⋅) that is associated with all MHC alleles.

FIG. 6D illustrates generating a presentation likelihood for a peptide p^{k} in association with MHC allele h using an example shared network model NN_{H}(⋅). As shown in FIG. 6D, the shared network model NN_{H}(⋅) receives the peptide sequence p^{k} and the MHC allele peptide sequence dₕ, and generates the output NN_{H}([p^{k}dₕ]). The output is mapped by function ƒ(⋅) to generate the estimated presentation likelihood uₖ.

### VIII.D.6. Allele-Noninteracting Variables

As discussed above, allele-noninteracting variables comprise information that influences presentation of peptides that are independent of the type of MHC allele. For example, allele-noninteracting variables may include protein sequences on the N-terminus and C-terminus of the peptide, the protein family of the presented peptide, the level of RNA expression of the source gene of the peptides, and any additional allele-noninteracting variables.

In one implementation, the training module 316 incorporates allele-noninteracting variables into the pan-allele presentation models in a similar manner as described with regard to the per-allele models and the multiple allele models. For example, in some embodiments, allele-noninteracting variables may be entered as inputs into a dependency function that is separate from the dependency function used for allele-interacting variables. In such embodiments, the outputs of the two separate dependency functions may be summed, and the resulting summation may be input into the transformation function to generate a presentation prediction.

### VIII.D.7. Multiple-Allele Samples

As described above, a test sample may contain multiple MHC alleles rather than a single MHC allele. In fact, a majority of samples taken from nature include more than one MHC allele. For example, each human genome contains six MHC class I loci. Therefore, a sample that contains a human genome can contain up to six different MHC class I alleles. Accordingly, samples that contain multiple MHC alleles, rather than a single MHC allele, are typical samples of real-life test cases.

In embodiments in which a test sample contains multiple MHC alleles, the pan-allele model may be employed to determine the probability that a given peptide from the test sample is presented by the multiple MHC alleles. However, as described briefly above, when using the pan-allele model to predict the likelihoods that a peptide will be presented by multiple MHC alleles, the pan-allele model described above is used iteratively for each MHC allele of the multiple MHC alleles. In other words, for each MHC allele of the multiple MHC alleles, the MHC allele peptide sequence and the peptide sequence are independently input into the dependency function shared by all MHC alleles. Based on these inputs, an output corresponding to the MHC allele is generated by the dependency function. This process is performed iteratively for each MHC allele of the multiple MHC alleles. Accordingly, each MHC allele of the multiple MHC alleles is independently associated with an output of the dependency function. The outputs associated with each MHC allele of the multiple MHC alleles are then combined.

The outputs of the dependency function that are associated with each MHC allele of the multiple MHC alleles can be combined. The manner in which the multiple outputs of the dependency function are combined can vary. For example, in some embodiments, the outputs of the dependency function iterations may be summed, and the resulting summation may be input into a transformation function to generate a presentation prediction. An equation that captures such an embodiment can be written as:

$${u}_{k}^{h} = Pr ( {p}^{k} presented ; MHC allele h ) = f ( {\sum }_{h = 1}^{T} {g}_{H} ( [ {p}^{k} {d}_{h} ] ; {θ}_{H} ) ) , \qquad ( 27 )$$

where T is the total number of unique MHC alleles in a sample containing multiple alleles. In alternative embodiments, the each individual output of the dependency function iterations may be input into a transformation function, and the resulting outputs from the transformation functions may be summed to generate a presentation prediction. An equation that captures this alternative embodiment can be written as:

$${u}_{k}^{h} = Pr ( {p}^{k} presented ; MHC allele h ) = ( {\sum }_{h = 1}^{T} f ( {g}_{H} ( [ {p}^{k} {d}_{h} ] ; {θ}_{H} ) ) ) , \qquad ( 28 )$$

Such embodiments, as well as others, in which multiple outputs of the dependency function are combined to predict the probability that a peptide will be presented in a multiple-allele setting are described herein.

### VIII.D.8. Training of Pan-Allele Models

Training a pan-allele model involves optimizing values for each parameter of the shared set of parameters θ_{H} associated with the dependency function. Specifically, the parameters θ_{H} are optimized such that the dependency function is able to output dependency scores that accurately indicate whether given MHC allele(s) will present a given peptide sequence.

To optimize the values of the parameters θ_{H}, the training data 170 is used. As mentioned above, the training data 170 used to train the model can include training samples that contain cells expressing single MHC alleles, training samples that contain cells expressing multiple MHC alleles, or training samples that contain cells expressing a combination of both single MHC alleles and multiple MHC alleles. Accordingly, each data instance i from the training data 170 is input into the pan-allele model, and more specifically, into the dependency function of the pan-allele model. For example, in certain embodiments, an MHC allele peptide sequence and a peptide sequence may be input into the pan-allele model. The pan-allele model then processes these inputs as if the model were being routinely used. However, unlike during the operation of the pan-allele model, during training of the pan-allele model, the known outcome of the peptide presentation is also input into the model. In other words, the label yⁱ is also input into the model. In embodiments in which the training sample input into the pan-allele model contains cells expressing multiple MHC alleles, yⁱ is set to 1 for each allele of the multiple MHC alleles in the sample.

After each iteration of the pan-allele model using a data instance i, the model determines the difference between the predicted probability of the MHC allele presenting the peptide and the known label yⁱ. Then, to minimize this difference, the pan-allele model modifies the parameters θ_{H}. In other words, the pan-allele model determines values for the parameters θ_{H} by minimizing the loss function with respect to OH. When the pan-allele model achieves a certain level of prediction accuracy, the training is complete and the model is ready for use.

### IX. Prediction Module

The prediction module 320 receives sequence data (e.g., infectious disease-derived peptide sequence data and/or HLA sequence data for a patient) and selects candidate antigens in the sequence data using the multi-part presentation models. Specifically, the sequence data may be DNA sequences, RNA sequences, and/or protein sequences. The prediction module 320 processes the sequence data into a plurality of peptide sequences p^{k} having 8-15 amino acids. For example, the prediction module 320 may process the given sequence “IEFROEIFJEF into three peptide sequences having 9 amino acids “IEFROEIFJ,” “EFROEIFJE,” and “FROEIFJEF.”

The presentation module 320 applies one or more of the multi-part presentation models to at least the processed peptide sequences to estimate presentation likelihoods of the peptide sequences (e.g., infectious disease-derived peptide sequences). Specifically, the prediction module 320 may select one or more candidate antigen peptide sequences that are likely to be presented on HLA molecules by applying the multi-part presentation models to the candidate antigens. In one implementation, the presentation module 320 selects candidate antigen sequences that have estimated presentation likelihoods above a predetermined threshold. In another implementation, the presentation model selects the N candidate antigen sequences that have the highest estimated presentation likelihoods.

In various embodiments, a therapeutic can be provided to the patient to induce immune responses. Example therapeutics include a vaccine composition comprising one or more selected antigens, a an engineered cell expressing a TCR or CAR specific for one or more selected antigen, or an antibody (e.g., a bispecific antibody) exhibiting binding specificity for one or more selected antigen.

### XI. Example Computer

FIG. 7 illustrates an example computer for implementing the entities shown in FIGS. 1 and 3A. The computer 700 includes at least one processor 702 coupled to a chipset 704. The chipset 704 includes a memory controller hub 720 and an input/output (I/O) controller hub 722. A memory 706 and a graphics adapter 712 are coupled to the memory controller hub 720, and a display 718 is coupled to the graphics adapter 712. A storage device 708, an input device 714, and network adapter 716 are coupled to the I/O controller hub 722. Other embodiments of the computer 700 have different architectures.

The storage device 708 is a non-transitory computer-readable storage medium such as a hard drive, compact disk read-only memory (CD-ROM), DVD, or a solid-state memory device. The memory 706 holds instructions and data used by the processor 702. The input interface 714 is a touch-screen interface, a mouse, track ball, or other type of pointing device, a keyboard, or some combination thereof, and is used to input data into the computer 700. In some embodiments, the computer 700 may be configured to receive input (e.g., commands) from the input interface 714 via gestures from the user. The graphics adapter 712 displays images and other information on the display 718. The network adapter 716 couples the computer 700 to one or more computer networks.

The computer 700 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 708, loaded into the memory 706, and executed by the processor 702.

The types of computers 700 used by the entities of FIG. 1 can vary depending upon the embodiment and the processing power required by the entity. For example, the presentation identification system 160 can run in a single computer 700 or multiple computers 700 communicating with each other through a network such as in a server farm. The computers 700 can lack some of the components described above, such as graphics adapters 712, and displays 718.

### EXAMPLES

### Example 1: Multi-Part Presentation Model Accurately Predicts Presentation of Viral Epitopes

Various multi-part presentation models were constructed and evaluated for their ability to predict likely presentation of viral epitopes. Specifically, three viruses were selected including human immunodeficiency virus (HIV), influenza A virus (IAV), and SARS-COV-2. These three viruses are well documented in the literature. T-cell validated epitopes were aggregated from the multiple sources, including the literature, the Immune Epitope Database (IEDB), as well as virus-specific databases like the Los Alamos National Laboratory HIV CD8 T-cell epitope database. The Immune Epitope Database (IEDB) is described in further detail in Vita R, et al, The Immune Epitope Database (IEDB): 2018 update. Nucleic Acids Res. 2018 Oct. 24, which is incorporated by reference in its entirety. The Los Alamos National Laboratory HIV CD8 T-cell epitope database is described in further detail in Llano, A., et al., The 2019 Optimal HIV CTL epitopes update: Growing diversity in epitope length and HLA restriction. HIV Molecular Immunology 2019, 3-27, which is incorporated by reference in its entirety.

A first multi-part model, referred to herein as the “ViralEDGE” model, was a 10 pan-specific, 10 allele-specific model ensemble trained with human mass spectrometry data and all of IEDB's binding affinity data from infectious diseases. The architecture of the ViralEDGE model is described herein in FIG. 4B. For each virus chosen, the ViralEDGE model was further re-trained with all epitopes from these three viruses' taxa removed. Synthetic negatives per-positive were generated in the binding affinity training and validation dataset. Specifically, a re-trained ViralEDGE model without any HIV, IAV, and SARS epitopes in its training set and with n=0 synthetic negatives per-positive is referred to herein as the “New Viral EDGE (0 Synth Neg)” model. A re-trained ViralEDGE model without any HIV, IAV, and SARS epitopes in its training set and with n=50 synthetic negatives per-positive is referred to herein as the “New Viral EDGE (50 Synth Neg)” model. A re-trained ViralEDGE model without any HIV, IAV, and SARS epitopes in its training set and with n=100 synthetic negatives per-positive is referred to herein as the “New Viral EDGE (100 Synth Neg)” model.

Additional, previously published models were further constructed, including a presentation model previously trained and deployed for predicting presentation of cancer epitopes (hereafter referred to as the “EDGE” model). This EDGE model does not include a multi-part presentation model and is trained on human immunopeptidomics. Further details of the EDGE model is described in U.S. Pat. No. 10,055,540 (see FIG. 13B showing predictions of “MS” model). An additional previously published model, referred to as the MHCFlurry model, was further constructed. The MHCflurry model incorporates both binding affinity and mass spectrometry data to train the model. Further details of the MHCFlurry model is described in O'Donnell, T. J., et al., MHCflurry 2.0: Improved Pan-Allele Prediction of MHC Class I-Presented Peptides by Incorporating Antigen Processing. Cell Systems, 11 (1), 42-48.e7, which is hereby incorporated by reference in its entirety.

For each evaluation virus chosen, the UniProt reference proteome (The UniProt Consortium. (2014). UniProt: a hub for protein information. Nucleic Acids Research, 43(D1), D204-212) was used to generate a reference proteome for that virus. Furthermore, 8-11mer epitopes were generated for each protein sequence, dropping any duplicated epitopes due to alternative polyprotein representations. The epitopes were analyzed by a variety of models to predict likely presentation by various class I HLA alleles.

Each of the models (e.g., EDGE, ViralEDGE, New Viral EDGE (0 Synth Neg), New Viral EDGE (50 Synth Neg), New Viral EDGE (100 Synth Neg), and MHCFlurry) were deployed to predict for each allele. An epitope-HLA prediction was treated as true if that epitope-HLA pair were reported as CD8 positive in any of the validation sources collected, all other predictions were assumed to be false.

Because each virus had a differing number of proteins, validated epitopes, and HLA alleles with validated epitopes to go with them, each benchmark was interpreted in total on the set of all HLA alleles which had at least one validated epitope on that dataset, per-allele for the top 5 alleles ranked by number of validated epitopes for that virus, or taking the top 5, 10, 20, or 25 alleles in aggregate to detect any allele-specific biases in either prediction or in validation. Models were evaluated according to precision recall curves and AUC values, as well as the number of positives in the top 500 predictions ranked by model score.

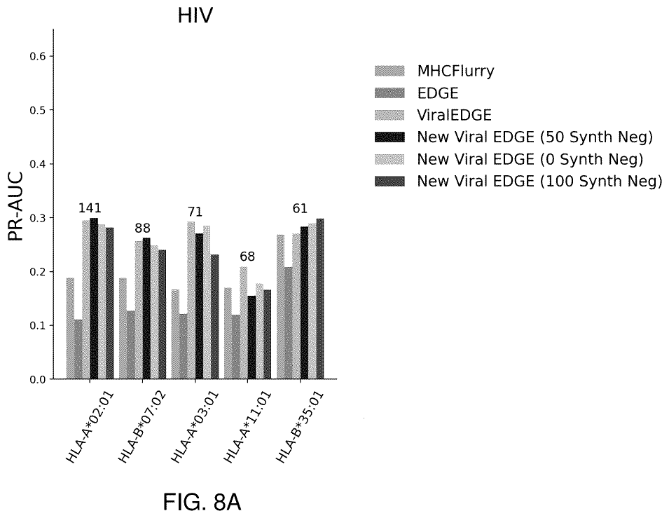

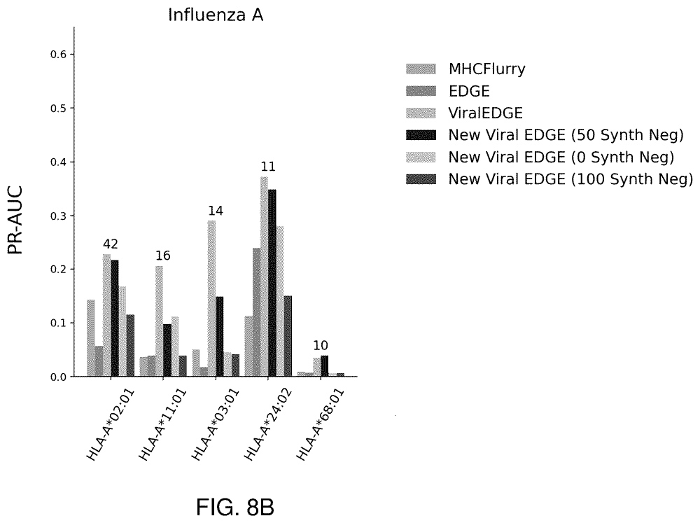

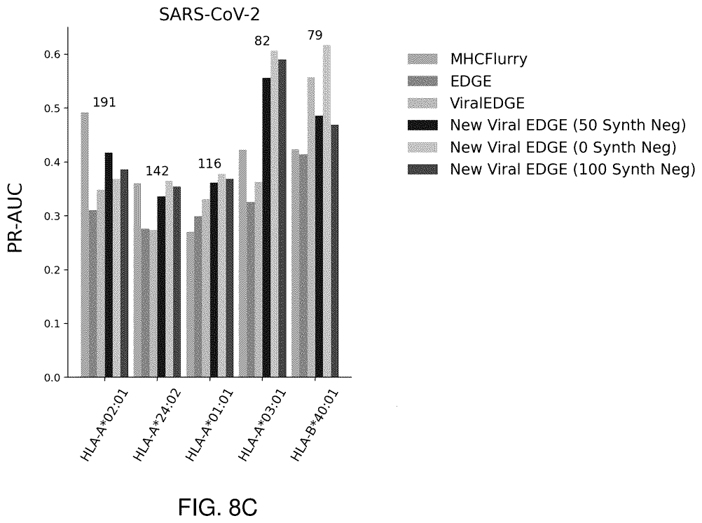

Reference is now made to FIGS. 8A-8C, which show the performance of the various models. For each of FIGS. 8A-8C, the bar plots for each allele show the performance of, from left to right, the MHCFlurry model, EDGE model, ViralEDGE model, New Viral EDGE (50 Synth Neg) model, New Viral EDGE (0 Synth Neg) model, and New Viral EDGE (100 Synth Neg) model.

Specifically, FIG. 8A shows performance of various models for predicting presentation of HIV epitopes across the top 5 class I HLA alleles. Here, the top 5 class I HLA alleles included HLA-A*02:01, HLA-B*07:02, HLA-A*03:01, HLA-A*11:01, and HLA-B*35:01. Generally, the multi-part presentation models (ViralEDGE, New Viral EDGE (0 Synth Neg), New Viral EDGE (50 Synth Neg), New Viral EDGE (100 Synth Neg)) achieved higher area under precision recall curve (PR-AUC) in comparison to the two previously published models (MHCFlurry and EDGE) across the 5 class I HLA alleles.

FIG. 8B shows performance of various models for predicting presentation of Influenza A epitopes across the top 5 alleles. Here, the top 5 class I HLA alleles included HLA-A*02:01, HLA-A*11:01, HLA-A*03:01, HLA-A*24:02, and HLA-A*68:01. Similar to results of HIV epitopes, the multi-part presentation models (ViralEDGE, New Viral EDGE (0 Synth Neg), New Viral EDGE (50 Synth Neg), New Viral EDGE (100 Synth Neg)) achieved higher area under precision recall curve (PR-AUC) in comparison to the two previously published models (MHCFlurry and EDGE) across the 5 class I HLA alleles.

FIG. 8C shows performance of various models for predicting presentation of SARS-CoV-2 epitopes across the top 5 alleles. Here, at least in relation to the HLA-A*01:01, HLA-A*03:01, and HLA-B*40:01 alleles, the multi-part presentation models (ViralEDGE, New Viral EDGE (0 Synth Neg), New Viral EDGE (50 Synth Neg), New Viral EDGE (100 Synth Neg)) achieved higher area under precision recall curve (PR-AUC) in comparison to the two previously published models (MHCFlurry and EDGE). For two of the alleles (HLA-A*02:01 and HLA-A*24:02), the multi-part presentation models (ViralEDGE, New Viral EDGE (0 Synth Neg), New Viral EDGE (50 Synth Neg), New Viral EDGE (100 Synth Neg)) achieved comparable PR-AUC values in comparison to the two previously published models (MHCFlurry and EDGE).

Generally, FIGS. 8A-8C show that the various multi-part presentation models generally outperform the previously published models in predicting presentation of different viral epitopes (e.g., HIV, Influenza A, and SARS-CoV-2).

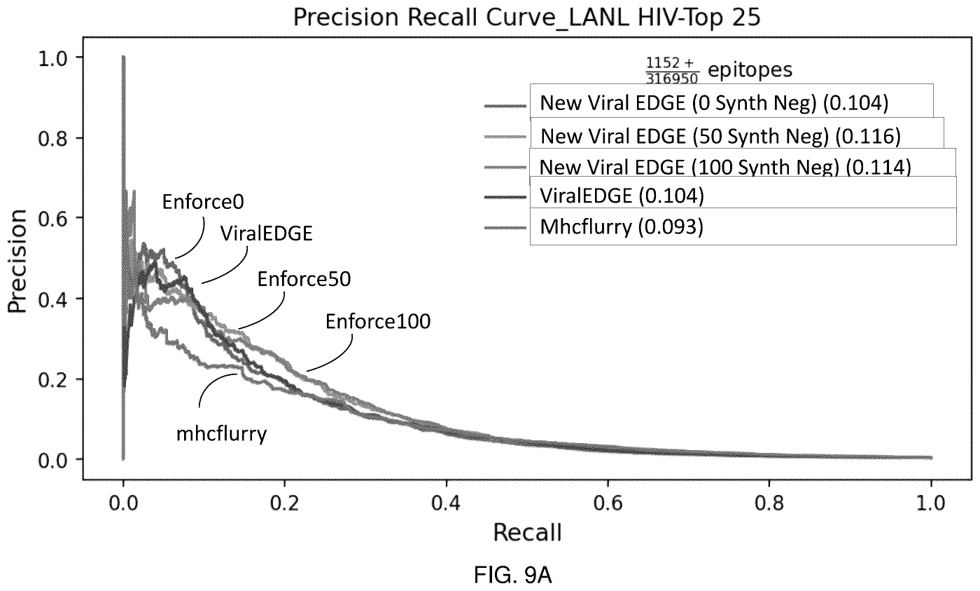

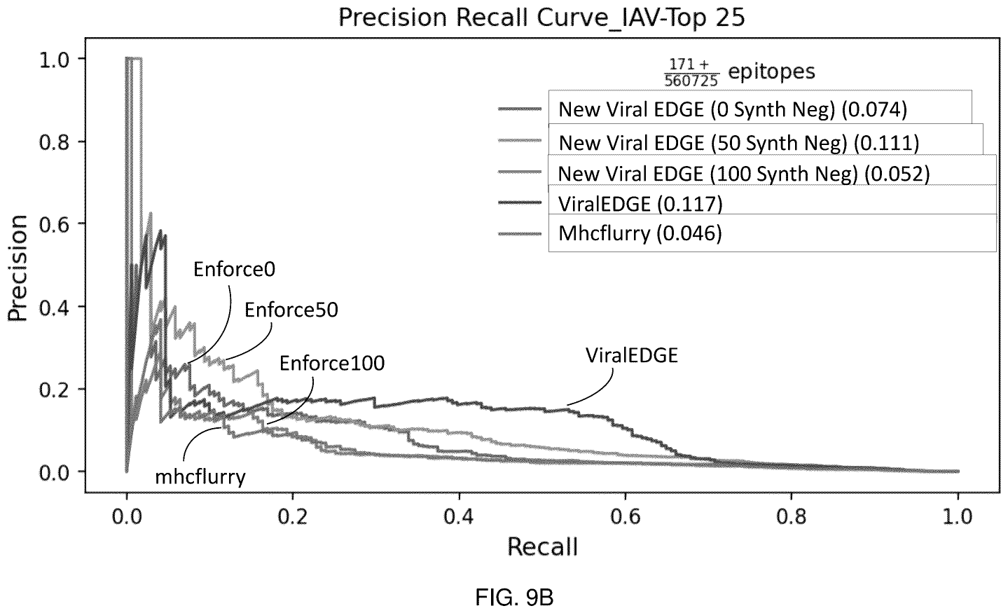

Reference is further made to FIG. 9A, which shows precision recall curves of various models for predicting presentation of HIV epitopes across the top 25 alleles, as well as FIG. 9B, which shows precision recall curves of various models for predicting presentation of Influenza A epitopes across the top 25 alleles. As shown in both FIGS. 9A and 9B, the multi-part presentation models (ViralEDGE, New Viral EDGE (0 Synth Neg), New Viral EDGE (50 Synth Neg), New Viral EDGE (100 Synth Neg)) achieved comparable or improved PR-AUC values in comparison to the previously published MHCFlurry model.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents, and patent applications cited within the body of the instant specification are hereby incorporated by reference in their entirety, for all purposes.

1. A method for identifying one or more infectious disease-derived antigens likely to be presented by cells of a subject, the method comprising:
obtaining peptide sequences of a plurality of infectious disease-derived antigens;
obtaining sequences of one or more MHC alleles of the subject;
inputting the peptide sequences of the plurality of infectious disease-derived antigens and the sequences of one or more MHC alleles of the subject into a multi-part presentation model to generate a set of numerical likelihoods that the plurality of infectious disease-derived antigens are presented by the one or more MHC alleles expressed on surfaces of cells of the subject,
wherein a first part of the multi-part presentation model comprises a pan-allele model portion that receives, as input, peptide sequences of one or more infectious disease-derived antigens and the sequences of one or more MHC alleles of the subject, or representations thereof, and
wherein a second part of the multi-part presentation model comprises a plurality of allele-specific models that each receives, as input, the peptide sequences of the plurality of infectious disease-derived antigens, or a representation thereof; and
selecting a subset of the plurality of infectious disease-derived antigens based on the set of numerical likelihoods to generate a set of selected antigens.

2. The method of claim 1, wherein the multi-part presentation model comprises a plurality of parameters generated using at least 1) mass spectrometry data and 2) binding affinity data determined from a plurality of samples.

3. The method of claim 1, wherein the multi-part presentation model comprises a plurality of parameters generated using a training dataset comprising:
training peptide sequences, and
for one or more of the training peptide sequences, a label derived from mass spectrometry data indicating whether the training peptide sequence was presented by one or more class I MHC alleles present in a plurality of samples.

4. The method of claim 3, wherein the training peptide sequences are identified through mass spectrometry on isolated peptides eluted from MHC alleles present in the plurality of samples.

5. The method of claim 3, wherein the multi-part presentation model comprises a plurality of parameters generated using a training dataset comprising:
for one or more of the training peptide sequences, a label derived from binding affinity data indicating whether the training peptide sequence was bound with one or more class I MHC alleles present in a plurality of samples.

6-8. (canceled)

9. The method of claim 1, wherein the pan-allele model portion comprises a neural network.

10. The method of claim 9, wherein a first set of layers of the neural network of the pan-allele model portion performs a dimensional reduction of the sequences of one or more MHC alleles of the subject.

11. The method of claim 9, wherein a second set of layers of the neural network of the pan-allele model portion receives, as input, a representation of the peptide sequences of the plurality of infectious disease-derived antigens and a dimensionally reduced representation of the sequences of one or more MHC alleles of the subject.

12. The method of claim 11, wherein the representation of the peptide sequences of the plurality of infectious disease-derived antigens is generated by encoding the peptide sequences via a one-hot encoding scheme.

13. The method of claim 11, wherein the second set of layers of the neural network models interactions between the peptide sequences of the plurality of infectious disease-derived antigens and the sequences of one or more MHC alleles of the subject.

14. The method of claim 1, wherein one or more of the allele-specific models comprise a neural network.

15. The method of claim 14, wherein the neural network of the allele-specific network receives, as input, a representation of the peptide sequences of the plurality of infectious disease-derived antigens, and outputs per-allele presentation likelihoods for an allele.

16. The method of claim 15, wherein the representation of the peptide sequences of the plurality of infectious disease-derived antigens is generated by encoding the peptide sequences via a one-hot encoding scheme.

17-21. (canceled)

22. The method of claim 1, wherein the cells of the subject comprise cells infected with one of a pathogen, virus, bacteria, fungus, or a parasite.

23. The method of claim 1, wherein infectious disease-derived antigens originate from one of a pathogen, virus, bacteria, fungus, or a parasite.

24. The method of claim 1, wherein infectious disease-derived antigens originate from an infectious disease organism selected from the group consisting of: severe acute respiratory syndrome-related coronavirus (SARS), severe acute respiratory syndrome coronavirus 2 (SARS-CoV-2), Ebola, HIV, Hepatitis B virus (HBV), influenza, Hepatitis C virus (HCV), Human papillomavirus (HPV), Cytomegalovirus (CMV), Chikungunya virus, Respiratory syncytial virus (RSV), Dengue virus, a orthymyxoviridae family virus, tuberculosis, pancorona, herpes simplex virus infection (HSV), flu, metapneumovirus (MPV), and Parainfluenza Viruses (PIVs).

25. A method of treating a subject for an infectious disease comprising performing claim 1, and further comprising obtaining a vaccine comprising the set of selected antigens, and administering the vaccine to the subject.

26-27. (canceled)

28. A method of manufacturing a vaccine, comprising performing claim 1, and further comprising producing or having produced a vaccine comprising the set of selected antigens.

29. A vaccine comprising a set of selected antigens selected by performing the method claim 1.

30-33. (canceled)

34. A non-transitory computer readable medium comprising instructions that, when executed by a processor, cause the processor to:
obtain peptide sequences of a plurality of infectious disease-derived antigens;
obtain sequences of one or more MHC alleles of the subject;
input the peptide sequences of the plurality of infectious disease-derived antigens and the sequences of one or more MHC alleles of the subject into a multi-part presentation model to generate a set of numerical likelihoods that the plurality of infectious disease-derived antigens are presented by the one or more MHC alleles expressed on surfaces of cells of the subject,
wherein a first part of the multi-part presentation model comprises a pan-allele model portion that receives, as input, peptide sequences of one or more infectious disease-derived antigens and the sequences of one or more MHC alleles of the subject, or representations thereof, and
wherein a second part of the multi-part presentation model comprises a plurality of allele-specific models that each receives, as input, the peptide sequences of the plurality of infectious disease-derived antigens, or a representation thereof; and
select a subset of the plurality of infectious disease-derived antigens based on the set of numerical likelihoods to generate a set of selected antigens.

35-57. (canceled)
